(19) United States
(12) Patent Application Publication
(10) Pub. No.: US 2026/0055171 A1
(43) Pub. Date: Feb. 26, 2026

(54) VEGF ANTAGONISTS AND METHODS OF USE THEREOF

(71) Applicant: Regeneron Pharmaceuticals, Inc., Tarrytown, NY (US)

(72) Inventors: Paige SOLOMON, Tarrytown, NY (US); Nianshuang WANG, Greenwich, CT (US); Yang SHEN, Scarsdale, NY (US); William OLSON, Yorktown Heights, NY (US); Chia-Yang LIN, Scarsdale, NY (US); Samuel DAVIS, New York, NY (US)

(73) Assignee: Regeneron Pharmaceuticals, Inc., Tarrytown, NY (US)

(21) Appl. No.: 19/234,063

(22) Filed: Jun. 10, 2025

Related U.S. Application Data
(60) Provisional application No. 63/658,638, filed on Jun. 11, 2024.

(51) Int. Cl. C07K 16/22; A61K 9/00; A61K 38/00; A61K 39/00; C07K 14/71

(52) U.S. Cl. CPC C07K 16/22; A61K 9/0043; C07K 14/71; A61K 38/00; A61K 2039/505; A61K 2039/54; C07K 2317/524; C07K 2317/526; C07K 2317/55; C07K 2317/622; C07K 2317/71; C07K 2317/76; C07K 2317/92; C07K 2319/00

(57) ABSTRACT

The present disclosure relates to VEGF antagonists. VEGF antagonists disclosed herein comprise a VEGF binding domain and a multimerization domain. Certain VEGF antagonists disclosed herein comprise five or more dimers, each dimer comprising two polypeptides, each polypeptide comprising a VEGF binding domain and a multimerization domain comprising an IgG Fc domain and, optionally, an IgM tailpiece. The disclosure further provides pharmaceutical compositions comprising the VEGF antagonists and methods of use of the VEGF antagonists in therapy, e.g., for treating angiogenic eye disorders. Also disclosed are nucleic acids encoding the VEGF antagonists, recombinant cells that express the VEGF antagonists, and methods of producing the VEGF antagonists.

### 1. CROSS-REFERENCE TO RELATED APPLICATIONS

This application claims the priority benefit of U.S. provisional application No. 63/658,638, filed on Jun. 11, 2024, the contents of which are incorporated herein in their entirety by reference thereto.

### 1.1 Sequence Listing

The instant application contains a Sequence Listing which has been submitted electronically in XML format and is hereby incorporated by reference in its entirety. Said XML copy, created on Aug. 21, 2025, is named RGN-046US_SL.xml and is 100,838 bytes in size.

### 2. BACKGROUND

Several eye disorders are associated with pathological angiogenesis. For example, the development of age-related macular degeneration (AMD) is associated with a process called choroidal neovascularization (CNV). Leakage from the CNV causes macular edema and collection of fluid beneath the macula resulting in vision loss. Diabetic macular edema (DME) is another eye disorder with an angiogenic component. DME is the most prevalent cause of moderate vision loss in patients with diabetes and is a common complication of diabetic retinopathy, a disease affecting the blood vessels of the retina. Yet another eye disorder associated with abnormal angiogenesis is central retinal vein occlusion (CRVO). CRVO is caused by obstruction of the central retinal vein that leads to a back-up of blood and fluid in the retina. The retina can also become ischemic, resulting in the growth of new, inappropriate blood vessels that can cause further vision loss and more serious complications. Release of vascular endothelial growth factor (VEGF) contributes to increased vascular permeability in the eye and inappropriate new vessel growth. Thus, inhibiting the angiogenic-promoting properties of VEGF appears to be an effective strategy for treating angiogenic eye disorders.

FDA-approved therapeutics for treatment of angiogenic eye disorders include the anti-VEGF antibody ranibizumab and VEGF antagonist (or “VEGF Trap”) aflibercept, each of which is administered via intravitreal (IVT) injection. Given that many patients suffering from eye disorders such as AMD require regular IVT administrations of such VEGF-inhibitory therapeutics for the rest of their lives, such treatments can provide significant discomfort and inconvenience to patients and burden to physicians and caregivers. There remains a need for VEGF antagonists having improved pharmacokinetic properties, allowing for smaller injection volumes per administration and/or less frequent dosing while maintaining a high level of efficacy.

### 3. SUMMARY

The present disclosure relates to VEGF antagonists comprising a VEGF binding domain and a multimerization domain. In particular, the VEGF antagonists of the disclosure generally comprise (a) a VEGF binding domain, and (b) a multimerization domain comprising an IgG Fc domain and, optionally, an IgM tailpiece. The VEGF binding domain may comprise an anti-VEGF antibody (or fragment or VEGF binding portion thereof) or may comprise a VEGF binding portion of a VEGF receptor (e.g., VEGFR1 and/or VEGFR2). In certain aspects, the VEGF binding domain comprises an Ig-like domain 2 of VEGFR1 and an Ig-like domain 3 of VEGFR2. Where the VEGF antagonists comprise an IgM tailpiece, such VEGF antagonists are generally multimers comprising at least five, typically six, dimers, and are often referred to herein as “hexameric VEGF antagonists” for convenience. The presence of IgM tailpieces allows the multimerization domains to assemble into polymeric structures via disulfide bonds involving a cysteine residue in the tailpiece. Multimerization domains comprising an IgG Fc domain and IgM tailpiece (in some cases described here in as an “Fc unit” for convenience), are depicted schematically in FIG. 1A (single Fc unit) and FIG. 1B (multimerization domain hexamer, also described herein as an “Fc core” for convenience). Hexameric VEGF antagonists of the disclosure are depicted schematically in FIG. 1C (dimer) and FIG. 1D (hexameric VEGF antagonist).

Exemplary VEGF antagonists are disclosed in Section 5.2 and numbered embodiments 1 to 194. Exemplary VEGF binding domains are disclosed in Section 5.3. Exemplary multimerization domains are disclosed in Sections 5.4 and subsections thereof. Exemplary linkers, e.g., useful for connecting the multimerization domains to the VEGF binding domains or components thereof are described in Section 5.5.

The disclosure further provides nucleic acids encoding the VEGF antagonists of the disclosure. The nucleic acids can be in the form of a single nucleic acid (e.g., a vector encoding two or more polypeptide chains) or a plurality of nucleic acids (e.g., two or more vectors encoding different polypeptide chains). The disclosure further provides host cells and cell lines engineered to express the nucleic acids and VEGF antagonists the disclosure, as well as methods of producing the VEGF antagonists. Exemplary nucleic acids, host cells, cell lines, and production methods are described in Section 5.7 and numbered embodiments 204 to 207.

The disclosure further provides compositions, e.g., populations of proteins and pharmaceutical compositions comprising the VEGF antagonists of the disclosure. Exemplary compositions are described in Section 5.8 and numbered embodiments 195 to 203.

Further described are methods of using VEGF antagonists of the disclosure, for example for treatment of angiogenic eye disorders. Exemplary methods for use are described in Section 5.9 and numbered embodiments 208 to 219.

Other features and advantages of aspects of VEGF antagonists of the present disclosure will become apparent from the following more detailed description, taken in conjunction with the accompanying drawings.

### 4. BRIEF DESCRIPTION OF THE FIGURES

FIGS. 1A-1D show the components and structures of exemplary constructs of the disclosure. FIG. 1A depicts an Fc unit comprising two polypeptides, each polypeptide comprising, in N- to C-terminal order, a hinge domain, an IgG Fc domain, and an IgM tailpiece. FIG. 1B depicts an Fc core comprising Fc units shown in FIG. 1A. FIG. 1C depicts an Fc unit as shown in FIG. 1A, further comprising a VEGF binding domain N-terminal to each Fc domain, described herein as a “VEGF antagonist dimer,” or simply “dimer” for convenience. The VEGF binding domain is depicted as comprising an Ig-like domain 2 of VEGFR1 (1) and an Ig-like domain 3 of VEGFR2 (2). FIG. 1D depicts a VEGF antagonist comprising six dimers. Such a construct comprising six dimers is often described as a “hexamer” or “hexameric VEGF antagonist” herein for convenience. Although the VEGF binding domains in FIGS. 1C and 1D are depicted as comprising an Ig-like domain 2 of VEGFR1 and an Ig-like domain 3 of VEGFR2, the VEGF binding domains can, in some cases, be in other formats, e.g., Fabs, scFvs, or other formats described in Section 5.3.

FIG. 2 shows the differences in assembly of six hexameric IgG-REGN3 constructs (Hex-REGN3) under non-reducing conditions. Lane 1: molecular weight marker; lane 2: Hex-REGN3 comprising Mut1-IgG4 us; lane 3: Hex-REGN3 comprising Mut1-IgG1; lane 4: Hex-REGN3 comprising Mut1+LALAPG-IgG1; lane 5: Hex-REGN3 comprising chimeric WT-IgG4 us; lane 6: Hex-REGN3 comprising WT-IgG1; lane 7: Hex-REGN3 comprising WT+LALAPG-IgG1.

FIGS. 3A-3C show size exclusion chromatography (SEC) profiles of three Hex-REGN3 constructs. FIG. 3A is a chromatogram of Hex-REGN3 (IgG1) Mut2 LALAPG. FIG. 3B is a chromatogram of Hex-REGN3 (IgG1) Mut1 LALAPG. FIG. 3C is a chromatogram of Hex-REGN3 (IgG1) Mut1 PVA/P329A.

FIGS. 4A-4D show VEGF binding kinetics of REGN3-Fc (“REGN3”; aflibercept) and three Hex-REGN3 constructs. FIG. 4A shows the binding kinetics of REGN3 to VEGF₁₆₅-Biotin. FIG. 4B shows the binding kinetics of Hex-REGN3 (IgG1) Mut1 LALAPG to VEGF₁₆₅-Biotin. FIG. 4C shows the binding kinetics of Hex-REGN3 (IgG1) Mut2 LALAPG to VEGF₁₆₅-Biotin. FIG. 4D shows the binding kinetics of Hex-REGN3 (IgG1) Mut1 PVA/P329A to VEGF₁₆₅-Biotin.

FIGS. 5A-5K show Fc receptor binding kinetics of REGN3-Fc (“REGN3”; aflibercept) and three Hex-REGN3 constructs. FIG. 5A shows the binding kinetics of REGN3 to hCD16a-Biotin. FIG. 5B shows the binding kinetics of REGN3 to hCD64-Biotin. FIG. 5C shows the binding kinetics of REGN3 to hFcRn-β2M-Biotin. FIG. 5D shows the binding kinetics of Hex-REGN3 (IgG1) Mut1 LALAPG to hCD16a-Biotin. FIG. 5E shows the binding kinetics of Hex-REGN3 (IgG1) Mut1 LALAPG to hCD64-Biotin. FIG. 5F shows the binding kinetics of Hex-REGN3 (IgG1) Mut1 LALAPG to hFcRn-β2M-Biotin. FIG. 5G shows the binding kinetics of Hex-REGN3 (IgG1) Mut1 PVA/P329A to hCD16a-Biotin. FIG. 5H shows the binding kinetics of Hex-REGN3 (IgG1) Mut1 PVA/P329A to hCD64-Biotin. FIG. 5I shows the binding kinetics of Hex-REGN3 (IgG1) Mut2 LALAPG to hCD16a-Biotin. FIG. 5J shows the binding kinetics of Hex-REGN3 (IgG1) Mut2 LALAPG to hCD64-Biotin. FIG. 5K shows the binding kinetics of Hex-REGN3 (IgG1) Mut2 LALAPG to hFcRn-β2M-Biotin.

FIGS. 6A-6B show the shift in REGN3 (IgG1) Mut1 LALAPG size upon VEGF binding. FIG. 6A shows the mass spectrophotometry of Hex-REGN3 without VEGF. FIG. 6B shows the mass spectrophotometry of Hex-REGN3 with VEGF.

FIGS. 7A-7E show the differences in stability of hexameric IgG1-Tailpiece (IgG1-T-Hex) constructs. FIG. 7A is a chromatogram of a freshly prepared sample of IgG1-T-Hex-WT. FIG. 7B is a chromatogram of a freshly prepared sample of IgG1-T-Hex-Mut1. FIG. 7C is a chromatogram of a sample of IgG1-T-Hex-WT that has been stored at 4° C. for over 30 days. FIG. 7D is a chromatogram of a sample of IgG1-T-Hex-Mut that has been stored at 4° C. for over 30 days. FIG. 7E displays the results of polyacrylamide gel electrophoresis of IgG1-T-Hex-WT and IgG1-T-Hex-Mut1 constructs under non-reducing (NR) and reducing (R) conditions.

FIGS. 8A-8B show results from evaluation of pharmacokinetics of fluorescently labeled Hex-REGN3 (IgG1) Mut1-V2 PVA/P329A. FIG. 8A shows concentration as measured by fluorescence on the indicated days following administration of 240419E (Hex-REGN3 (IgG1) Mut1-V2 PVA/P329A at a first molecule: dye ratio) in two different rabbits, 1055 and 1056. FIG. 8B shows concentration as measured by fluorescence on the indicated days following administration of 240419F (Hex-REGN3 (IgG1) Mut1-V2 PVA/P329A at a second molecule: dye ratio) in two different rabbits, 1053 and 1054. OD=oculus dexter (right eye) measurements. OS=oculus sinister (left eye) measurements. Half-lives (t_{1/2}) measured for each rabbit are shown based on the depicted trendline.

### 5. DETAILED DESCRIPTION

### 5.1. Definitions

About, Approximately: The terms “about”, “approximately” and the like are used throughout the specification in front of a number to show that the number is not necessarily exact (e.g., to account for fractions, variations in measurement accuracy and/or precision, timing, etc.). It should be understood that a disclosure of “about X” or “approximately X” where X is a number is also a disclosure of “X.” Thus, for example, a disclosure of an embodiment in which one sequence has “about X % sequence identity” to another sequence is also a disclosure of an embodiment in which the sequence has “X % sequence identity” to the other sequence.

And, or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

Angiogenic Eye Disorder: The term “angiogenic eye disorder” as used herein refers to any disease of the eye which is caused by or associated with the growth or proliferation of blood vessels or by blood vessel leakage. Non-limiting examples of angiogenic eye disorders include age-related macular degeneration (e.g., wet AMD, exudative AMD, etc.), retinal vein occlusion (RVO), central retinal vein occlusion (CRVO; e.g., macular edema following CRVO), branch retinal vein occlusion (BRVO), diabetic macular edema (DME), choroidal neovascularization (CNV; e.g., myopic CNV), iris neovascularization, neovascular glaucoma, post-surgical fibrosis in glaucoma, proliferative vitreoretinopathy (PVR), optic disc neovascularization, corneal neovascularization, retinal neovascularization, vitreal neovascularization, pannus, pterygium, vascular retinopathy, and diabetic retinopathies.

Antibody: The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, but is not limited to, monoclonal antibodies, human antibodies, humanized antibodies, camelized antibodies, chimeric antibodies, bispecific or multispecific antibodies and anti-idiotypic (anti-id) antibodies. The antibodies can be of any isotype/class (e.g., IgG, IgE, IgM, IgD, IgA and IgY) or subclass (e.g., IgG1, IgG2, IgG3, IgG4, IgA1 and IgA2). Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding domain or amino-terminus of the antibody. The N-terminus is a variable region and at the C-terminus is a constant region; the CH3 and CL domains represent the carboxy-terminus of the heavy and light chain, respectively, of natural antibodies. For convenience, and unless the context dictates otherwise, the reference to an antibody also refers to antibody fragments as well as engineered antibodies that include non-naturally occurring antigen-binding domains and/or antigen-binding domains having non-native configurations.

Associated: The term “associated” in the context of a VEGF antagonist refers to a functional relationship between two or more polypeptide chains or portions of a polypeptide chain. In particular, the term “associated” means that two or more polypeptides are associated with one another, e.g., non-covalently through molecular interactions or covalently through one or more disulfide bridges or chemical cross-linkages, so as to produce a functional antigen-binding molecule. Examples of associations that might be present in a VEGF antagonist of the disclosure include (but are not limited to) associations between homodimeric or heterodimeric Fc domains in an Fc region, associations between VH and VL regions in a Fab or scFv, associations between CH1 and CL in a Fab, and associations between CH3 and CH3 in a domain substituted Fab.

Complementarity Determining Region or CDR: The terms “complementarity determining region” or “CDR,” as used herein, refer to the sequences of amino acids within antibody variable regions which confer antigen specificity and binding affinity. In general, there are three CDRs in each heavy chain variable region (CDR-H1, CDR-H2, CDR-H3) and three CDRs in each light chain variable region (CDR1-L1, CDR-L2, CDR-L3). Exemplary conventions that can be used to identify the boundaries of CDRs include, e.g., the Kabat definition, the Chothia definition, the target binding domain definition and the IMGT definition. See, e.g., Kabat, 1991, “Sequences of Proteins of Immunological Interest,” National Institutes of Health, Bethesda, Md. (Kabat numbering scheme); Al-Lazikani et al., 1997, J. Mol. Biol. 273:927-948 (Chothia numbering scheme); Martin et al., 1989, Proc. Natl. Acad. Sci. USA 86:9268-9272 (target binding domain numbering scheme); and Lefranc et al., 2003, Dev. Comp. Immunol. 27:55-77 (IMGT numbering scheme). For example, for classic formats, under Kabat, the CDR amino acid residues in the heavy chain variable domain (VH) are numbered 31-35 (CDR-H1), 50-65 (CDR-H2), and 95-102 (CDR-H3); and the CDR amino acid residues in the light chain variable domain (VL) are numbered 24-34 (CDR-L1), 50-56 (CDR-L2), and 89-97 (CDR-L3). Under Chothia, the CDR amino acids in the VH are numbered 26-32 (CDR-H1), 52-56 (CDR-H2), and 95-102 (CDR-H3); and the amino acid residues in VL are numbered 26-32 (CDR-L1), 50-52 (CDR-L2), and 91-96 (CDR-L3). By combining the CDR definitions of both Kabat and Chothia, the CDRs consist of amino acid residues 26-35 (CDR-H1), 50-65 (CDR-H2), and 95-102 (CDR-H3) in human VH and amino acid residues 24-34 (CDR-L1), 50-56 (CDR-L2), and 89-97 (CDR-L3) in human VL. Under IMGT the CDR amino acid residues in the VH are numbered approximately 26-35 (CDR-H1), 51-57 (CDR-H2) and 93-102 (CDR-H3), and the CDR amino acid residues in the VL are numbered approximately 27-32 (CDR-L1), 50-52 (CDR-L2), and 89-97 (CDR-L3) (numbering according to “Kabat”). Under IMGT, the CDR regions of an antibody can be determined using the program IMGT/DomainGap Align. Public databases are available for identifying CDR sequences within an antibody.

EC50: The term “EC50” refers to the half maximal effective concentration of a molecule, such as a VEGF antagonist, which induces a response halfway between the baseline and maximum after a specified exposure time. The EC50 essentially represents the concentration of a molecule where 50% of its maximal effect is observed. Thus, reduced or weaker binding is observed with an increased EC50, or half maximal effective concentration value.

Effector Function: The term “effector function” refers to an activity of an antibody molecule that is mediated by binding through a domain of the antibody other than the antigen-binding domain, usually mediated by binding of effector molecules. Effector function includes complement-mediated effector function, which is mediated by, for example, binding of the C1 component of the complement to the antibody. Activation of complement is important in the opsonization and lysis of cell pathogens. The activation of complement also stimulates the inflammatory response and may also be involved in autoimmune hypersensitivity. Effector function also includes Fc receptor (FcR)-mediated effector function, which may be triggered upon binding of the constant domain of an antibody to an Fc receptor (FcR). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. An effector function of an antibody may be altered by altering, e.g., enhancing or reducing, the affinity of the antibody for an effector molecule such as an Fc receptor or a complement component. Binding affinity will generally be varied by modifying the effector molecule binding site, and in this case, it is appropriate to locate the site of interest and modify at least part of the site in a suitable way. It is also envisaged that an alteration in the binding site on the antibody for the effector molecule need not alter significantly the overall binding affinity but may alter the geometry of the interaction rendering the effector mechanism ineffective as in non-productive binding. It is further envisaged that an effector function may also be altered by modifying a site not directly involved in effector molecule binding, but otherwise involved in performance of the effector function.

Epitope: An epitope, or antigenic determinant, is a portion of an antigen recognized by an antibody or other antigen-binding moiety as described herein. An epitope can be linear or conformational. As described herein, an epitope may be described as “comprising a sequence” of a particular region (e.g., protein domain) of an antigen. Such description includes both linear epitopes and conformational epitopes, and describes epitopes comprising at least one amino acid present in the particular region of the antigen. Such epitopes may or may not comprise additional amino acids which are not present in the particular region.

Fab: The term “Fab” in the context of an antigen-binding molecule of the disclosure of the disclosure refers to a pair of polypeptide chains, the first comprising a variable heavy (VH) domain of an antibody N-terminal to a first constant domain (referred to herein as C1), and the second comprising variable light (VL) domain of an antibody N-terminal to a second constant domain (referred to herein as C2) capable of pairing with the first constant domain. In a native antibody, the VH is N-terminal to the first constant domain (CH1) of the heavy chain and the VL is N-terminal to the constant domain of the light chain (CL). The Fabs of the disclosure can be arranged according to the native orientation or include domain substitutions or swaps that facilitate correct VH and VL pairings. For example, it is possible to replace the CH1 and CL domain pair in a Fab with a CH3-domain pair to facilitate correct modified Fab-chain pairing in heterodimeric molecules. It is also possible to reverse CH1 and CL, so that the CH1 is attached to VL and CL is attached to the VH, a configuration generally known as Crossmab (a type of “domain exchanged” arrangement). Alternatively, or in addition to, the use of substituted or swapped constant domains, correct chain pairing can be achieved by the use of universal light chains that can pair with both variable regions of a heterodimeric antigen-binding molecule of the disclosure. The term “Fab” encompasses single chain Fabs.

Fc Core: The term “Fc core” (sometimes simply “core” for convenience) refers to a covalent or non-covalent assembly of at least five, in some cases six, Fc units.

Fc Domain and Fc Region: The term “Fc domain” refers to a portion of the heavy chain that pairs with the corresponding portion of another heavy chain. The term “Fc region” refers to the region of antibody-based binding molecules formed by association of two heavy chain Fc domains. The two Fc domains within the Fc region may be the same or different from one another. In a native antibody the Fc domains are typically identical, but one or both Fc domains might advantageously be modified to allow for heterodimerization, e.g., via a knob-in-hole interaction and/or for purification, e.g., via star mutations.

Fc Unit: The term “Fc unit” (sometimes simply “unit” for convenience) refers to an Fc dimer comprising two multimerization domains associated with one another, the multimerization domains comprising an IgG Fc domain and an IgM tailpiece.

Fv: The term “Fv” refers to the minimum antibody fragment derivable from an immunoglobulin that contains a complete target recognition and binding site. This region consists of a dimer of one heavy and one light chain variable domain in a tight, noncovalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target. The reference to a VH-VL dimer herein is not intended to convey any particular configuration. When present on a single polypeptide chain (e.g., a scFv), the VH and be N-terminal or C-terminal to the VL.

Hinge: The term “hinge”, as used herein, is intended to include the sequence of consecutive amino acid residues that connect the C-terminus of the CH1 to the N-terminus of the CH2 domain of an immunoglobulin. Several amino acids of the N-terminus of the CH2 domain, which are coded by the CH2 exon, are also considered part of the “lower hinge”. Without being bound by any one theory, amino acids of the hinge region of IgG1, IgG2 and IgG4 have been characterized as comprising 12-15 consecutive amino acids encoded by a distinct hinge exon, and several N-terminal amino acids of the CH2 domain (encoded by the CH2 exon) (Brekke et al., 1995, Immunology Today 16(2): 85-90). On the other hand, IgG3 comprises a hinge region consisting of four segments: one upper segment resembling the hinge region of IgG1, and 3 segments that are identical amino acid repeats unique to IgG3. The term “hinge” as used herein is not intended to be limited to naturally occurring hinge sequences, but includes non-naturally occurring hinge sequences such as a chimeric hinge sequence. Exemplary hinge sequences are set forth in Section 5.6.

Host Cell or Recombinant Host Cell: The terms “host cell” and “recombinant host cell” as used herein refer to a cell that has been genetically engineered, e.g., through introduction of a heterologous nucleic acid. It should be understood that such terms are intended to refer not only to the particular subject cell but to the progeny of such a cell. Because certain modifications can occur in succeeding generations due to either mutation or environmental influences, such progeny may not, in fact, be identical to the parent cell, but are still included within the scope of the term “host cell” as used herein. A host cell can carry the heterologous nucleic acid transiently, e.g., on an extrachromosomal heterologous expression vector, or stably, e.g., through integration of the heterologous nucleic acid into the host cell genome. For purposes of expressing an antigen-binding molecule, a host cell can be a cell line of mammalian origin or mammalian-like characteristics, such as monkey kidney cells (COS, e.g., COS-1, COS-7), HEK293, baby hamster kidney (BHK, e.g., BHK21), Chinese hamster ovary (CHO), NSO, PerC6, BSC-1, human hepatocellular carcinoma cells (e.g., Hep G2), SP2/0, HeLa, Madin-Darby bovine kidney (MDBK), myeloma and lymphoma cells, or derivatives and/or engineered variants thereof. The engineered variants include, e.g., glycan profile modified and/or site-specific integration site derivatives.

Multimerization domain: The term “multimerization domain” as used herein refers to a polypeptide chain or an amino acid sequence capable of facilitating an association between two or more polypeptide chains to form a multimer. A first multimerization domain can associate with an identical second multimerization domain, or can associate with a second multimerization domain that is different from the first. In some embodiments, a multimerization domain comprises an Fc domain (e.g., as described in Section 5.4.1), with the association of two Fc domains forming an Fc region. In some embodiments, a multimerization domain comprises a tailpiece (e.g., as described in Section 5.4.2), with the association of five or six tailpieces forming a pentamer or hexamer. In some embodiments, a multimerization domain comprises both an Fc domain and a tailpiece.

Operably Linked: The term “operably linked” as used herein refers to a functional relationship between two or more regions of a polypeptide chain in which the two or more regions are linked so as to produce a functional polypeptide, or two or more nucleic acid sequences, e.g., to produce an in-frame fusion of two polypeptide components or to link a regulatory sequence to a coding sequence. In the context of a fusion protein or other polypeptide, the term “operably linked” means that two or more amino acid segments are linked so as to produce a functional polypeptide. In the context of a nucleic acid encoding a fusion protein, such as an antigen-binding molecule of the disclosure, “operably linked” means that the two nucleic acids are joined such that the amino acid sequences encoded by the two nucleic acids remain in-frame. In the context of transcriptional regulation, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system.

Polypeptide, Peptide and Protein: The terms “polypeptide”, “peptide” and “protein” are used interchangeably herein to refer to a polymer of amino acid residues.

Single Chain Fab or scFab: The term “single chain Fab” or “scFab” as used herein refers to a target binding domain comprising a VH domain, a CH1 domain, a VL domain, a CL domain and a linker. In some embodiments, the foregoing domains and linker are arranged in one of the following orders in a N-terminal to C-terminal orientation: (a) VH-CH1-linker-VL-CL, (b) VL-CL-linker-VH-CH1, (c) VH-CL-linker-VL-CH1 or (d) VL-CH1-linker-VH-CL. Linkers are suitably noncleavable linkers of at least 30 amino acids, preferably between 32 and 50 amino acids. Single chain Fab fragments are typically stabilized via the natural disulfide bond between the CL domain and the CH1 domain. In addition, these single chain Fab molecules might be further stabilized by generation of interchain disulfide bonds via insertion of cysteine residues (e.g., at position 44 in the VH domain and position 100 in the VL domain according to Kabat numbering).

Single Chain Fv or scFv: The term “single chain Fv” or “scFv” as used herein refers to a polypeptide chain comprising the VH and VL domains of antibody, where these domains are present in a single polypeptide chain. Preferably, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds. (1994), Springer-Verlag, New York, pp. 269-315.

Subject: The term “subject” includes human and non-human animals. Non-human animals include all vertebrates, e.g., mammals and non-mammals, such as non-human primates, sheep, dog, cow, chickens, amphibians, and reptiles. In certain embodiments, the subject is human. Except when noted, the terms “patient” or “subject” are used herein interchangeably.

Tailpiece: The term “tailpiece” as used herein refers to an IgM tailpiece (T) sequence or a variant or functional equivalent thereof that, when operably linked to the C-terminus of an Fc domain, is capable of assembling individual Fc dimers into a covalent or non-covalent assembly of at least five, typically six, dimers. Exemplary tailpieces are described in Section 5.4.2.

Treat, Treatment, Treating: As used herein, the terms “treat”, “treatment” and “treating” refer to the reduction or amelioration of the progression, severity and/or duration of an angiogenic disorder, or the amelioration of one or more symptoms (preferably, one or more discernible symptoms) of an angiogenic disorder resulting from the administration of one or more VEGF antagonists of the disclosure. In specific embodiments, the terms “treat”, “treatment” and “treating” refer to the amelioration of at least one measurable physical parameter of an angiogenic disorder, not necessarily discernible by the patient. In other embodiments the terms “treat”, “treatment” and “treating” refer to the inhibition of the progression of an angiogenic disorder, either physically by, e.g., stabilization of a discernible symptom, physiologically by, e.g., stabilization of a physical parameter, or both.

Universal Light Chain, ULC: The term “universal light chain” or “ULC” as used herein in the context of an antigen-binding domain refers to a light chain polypeptide capable of pairing with the heavy chain region of the antigen-binding domain and also capable of pairing with other heavy chain regions. Universal light chains are also known as “common light chains.”

VEGF Antagonist: The term “VEGF antagonist” as used herein refers to a molecule that is capable of blocking, reducing or interfering with the normal biological activity of vascular endothelial growth factor (VEGF) or a VEGF receptor. VEGF antagonists include molecules which interfere with the interaction between VEGF and a natural VEGF receptor, e.g., molecules which bind to VEGF or a VEGF receptor and prevent or otherwise hinder the interaction between VEGF and a VEGF receptor. In some embodiments, the VEGF antagonist comprises at least one VEGF binding domain and at least one multimerization domain. In some embodiments, the VEGF antagonist comprises two or more polypeptide chains, each comprising a VEGF binding domain and a multimerization domain. The term “VEGF antagonist” encompasses a single polypeptide comprising a VEGF binding domain and a multimerization domain, as well as a multimeric molecule comprising 2, 3, 4, 5, 6, 7, 8, 9, 10, 11, or 12 (or more) such polypeptides.

VEGF Binding Domain: The term “VEGF binding domain” as used herein refers to a polypeptide domain that has the ability to bind to vascular endothelial growth factor (VEGF). The term includes antibodies that specifically bind to VEGF, or antigen binding domains or fragments thereof. “VEGF binding domain” also encompasses a VEGF binding portion of one or more VEGF receptors (e.g., VEGFR1 and/or VEGFR2). VEGF binding domains can be incorporated into VEGF antagonists of the disclosure. Exemplary VEGF binding domains are described in Section 5.3.

VH: The term “VH” refers to the variable region of an immunoglobulin heavy chain of an antibody, including the heavy chain of an Fv, scFv, dsFv or Fab.

VL: The term “VL” refers to the variable region of an immunoglobulin light chain, including the light chain of an Fv, scFv, dsFv or Fab.

### 5.2. VEGF Antagonists

The present disclosure relates to VEGF binding molecules comprising (a) a VEGF binding domain and (b) a multimerization domain comprising an Fc domain and, optionally, a tailpiece. Such VEGF binding molecules are often referred to herein as “VEGF antagonists.”

In certain embodiments, the VEGF antagonists are multimers comprising one or more dimers, each dimer comprising a polypeptide comprising a VEGF binding domain and a multimerization domain. Particular aspects are directed to VEGF antagonists comprising at least five, typically six, such dimers, often described herein as a “hexameric VEGF antagonist” or simply “hexamer” for convenience. Without being bound by theory, such hexameric VEGF antagonists are understood to have improved pharmacokinetics and potency relative to a corresponding VEGF antagonist comprising a single dimer. VEGF antagonists disclosed herein include monomers (comprising a single polypeptide), dimers (comprising two polypeptides), and higher order multimers (e.g., comprising 2, 3, 4, 5, 6, or more dimers).

In certain aspects, disclosed is a hexameric VEGF antagonist comprising five or more (e.g., six) dimers, each dimer comprising two polypeptides, each polypeptide comprising:
(a) a VEGF binding domain comprising (i) an Ig-like domain 2 of VEGFR1 comprising an amino acid sequence having at least 90% (e.g., at least 95%) sequence identity to SEQ ID NO: 13; and (ii) an Ig-like domain 3 of VEGFR2 comprising an amino acid sequence having at least 90% (e.g., at least 95%) sequence identity to SEQ ID NO:15 or SEQ ID NO: 50; and
(b) a multimerization domain comprising (i) an IgG Fc domain having at least 90% sequence identity to the amino acid sequence of SEQ ID NO:1; and (ii) an IgM tailpiece having at least 80% sequence identity to the amino acid sequence of SEQ ID NO:2.

In further aspects, disclosed is a VEGF antagonist comprising (a) a VEGF binding domain, and (b) a multimerization domain comprising a chimeric IgG Fc domain comprising an amino acid sequence that has at least 80% (e.g., at least 90%) sequence identity to SEQ ID NO: 1, provided that the chimeric multimerization domain has the sequence P-V-A-absent at amino acids 233 to 236 and the amino acid substitution P329A as compared to SEQ ID NO: 1, as defined by EU numbering (described in some cases as an “IgG1 PVA P329A” Fc domain). In some embodiments, the multimerization domain further comprises a tailpiece C-terminal to the chimeric IgG Fc domain.

Exemplary VEGF binding domains suitable for use in the VEGF antagonists of the disclosure are described in Section 5.3.

Exemplary multimerization domains suitable for use in the VEGF antagonists of the disclosure are described in Section 5.4 and comprise an Fc domain (e.g., an Fc domain as described in Section 5.4.1) and, optionally, a tailpiece (e.g., a tailpiece as described in Section 5.4.2). An Fc domain can include a hinge domain at its N-terminus. Exemplary hinge domains are described in Section 5.6.

Exemplary polypeptide chains of VEGF antagonists disclosed herein are provided in Table 1, below, with reference to:
the VEGF binding domain; and
the multimerization domain, including the Fc domain and, if present, the tailpiece.

TABLE 1
Exemplary Polypeptide Chains

| # | VEGF binding domain | Fc domain | Tailpiece |
|---|---|---|---|
| 1 | Ig-like domain 2 of VEGFR1 | IgG1-T-Hex(WT) | SEQ ID |
| | operably linked to Ig-like | (having at least 90%, at least at | NO: 2 (e.g., |
| | domain 3 of VEGFR2 | least 95%, at least 98%, at least | SEQ ID |
| | (e.g., as described in | 99%, or 100% sequence identity to | NO: 3 or SEQ |
| | Section 5.3.2) | SEQ ID NO: 5) | ID NO: 4) |
| 2 | Ig-like domain 2 of VEGFR1 | IgG1-T-Hex(Mut) | SEQ ID |
| | operably linked to Ig-like | (having at least 90%, at least at | NO: 2 (e.g., |
| | domain 3 of VEGFR2 | least 95%, at least 98%, at least | SEQ ID |
| | (e.g., as described in | 99%, or 100% sequence identity to | NO: 3 or SEQ |
| | Section 5.3.2) | SEQ ID NO: 6) | ID NO: 4) |
| 3 | Ig-like domain 2 of VEGFR1 | IgG1-T-Hex(Mut1) | SEQ ID |
| | operably linked to Ig-like | (having at least 90%, at least at | NO: 2 (e.g., |
| | domain 3 of VEGFR2 | least 95%, at least 98%, at least | SEQ ID |
| | (e.g., as described in | 99%, or 100% sequence identity to | NO: 3 or SEQ |
| | Section 5.3.2) | SEQ ID NO: 7) | ID NO: 4) |
| 4 | Ig-like domain 2 of VEGFR1 | IgG1-T-Hex(Mut2) | SEQ ID |
| | operably linked to Ig-like | (having at least 90%, at least at | NO: 2 (e.g., |
| | domain 3 of VEGFR2 | least 95%, at least 98%, at least | SEQ ID |
| | (e.g., as described in | 99%, or 100% sequence identity to | NO: 3 or SEQ |
| | Section 5.3.2) | SEQ ID NO: 8) | ID NO: 4) |
| 5 | Ig-like domain 2 of VEGFR1 | IgG1-T-Mut1 LALAPG | SEQ ID |
| | operably linked to Ig-like | (having at least 90%, at least at | NO: 2 (e.g., |
| | domain 3 of VEGFR2 | least 95%, at least 98%, at least | SEQ ID |
| | (e.g., as described in | 99%, or 100% sequence identity to | NO: 3 or SEQ |
| | Section 5.3.2) | SEQ ID NO: 9) | ID NO: 4) |
| 6 | Ig-like domain 2 of VEGFR1 | IgG1-T-Mut1 PVA/P329A | SEQ ID |
| | operably linked to Ig-like | (having at least 90%, at least at | NO: 2 (e.g., |
| | domain 3 of VEGFR2 | least 95%, at least 98%, at least | SEQ ID |
| | (e.g., as described in | 99%, or 100% sequence identity to | NO: 3 or SEQ |
| | Section 5.3.2) | SEQ ID NO: 10) | ID NO: 4) |
| 7 | Ig-like domain 2 of VEGFR1 | IgG1-T- Mut2 LALAPG | SEQ ID |
| | operably linked to Ig-like | (having at least 90%, at least at | NO: 2 (e.g., |
| | domain 3 of VEGFR2 | least 95%, at least 98%, at least | SEQ ID NO: |
| | (e.g., as described in | 99%, or 100% sequence identity to | 3 or SEQ ID |
| | Section 5.3.2) | SEQ ID NO: 11) | NO: 4) |
| 8 | Ig-like domain 2 of VEGFR1 | IgG4/IgG1-T-Mut1 | SEQ ID |
| | operably linked to Ig-like | (having at least 90%, at least at | NO: 2 (e.g., |
| | domain 3 of VEGFR2 | least 95%, at least 98%, at least | SEQ ID |
| | (e.g., as described in | 99%, or 100% sequence identity to | NO: 3 or SEQ |
| | Section 5.3.2) | SEQ ID NO: 12) | ID NO: 4) |
| 9 | Anti-VEGF antibody or | hIgG1 PVA/P329A Fc | None |
| | fragment or component | (having at least 90%, at least at | |
| | thereof | least 95%, at least 98%, at least | |
| | (e.g., as described in | 99%, or 100% sequence identity to | |
| | Section 5.3.3) | SEQ ID NO: 26 | |
| 10 | Anti-VEGF antibody or | IgG1-T-Mut1 PVA/P329A | SEQ ID |
| | fragment or component | (having at least 90%, at least at | NO: 2 (e.g., |
| | thereof | least 95%, at least 98%, at least | SEQ ID |
| | (e.g., as described in | 99%, or 100% sequence identity to | NO: 3 or SEQ |
| | Section 5.3.3) | SEQ ID NO: 10) | ID NO: 4) |

It will be understood that the exemplary polypeptides described in Table 1 can include one or more further modifications in addition to those specifically mentioned, e.g., one or more mutations that modify effector function, e.g., as described in Section 5.4.1, and/or one or more mutations that promote heterodimerization and/or facilitate purification (e.g., “star” mutations), e.g., as described in Section 5.4.1.4.

### 5.3. VEGF Binding Domains

The VEGF antagonists of the disclosure comprise one or more VEGF binding domains (e.g., one, two, three, four, or more VEGF binding domains). The VEGF binding domain may be any domain or region of a polypeptide capable of specifically binding to vascular endothelial growth factor (VEGF). In some embodiments, a VEGF antagonist of the disclosure comprises a single VEGF binding domain.

In some embodiments, the VEGF antagonist comprises two or more VEGF binding domains. Generally, where a VEGF antagonist comprises two or more polypeptides, each polypeptide comprises a VEGF binding domain. The two or more VEGF binding domains can be identical, or can be different. In some embodiments, the two or more VEGF binding domains are identical, and bind the same epitope on VEGF. In other embodiments, the two or more target binding domains are different, and bind different epitopes on VEGF.

In some embodiments, the VEGF binding domain is fused directly to the multimerization domain. In some embodiments, the VEGF binding domain is operably linked to a multimerization domain via a linker. Exemplary linkers are set forth in Section 5.5.

In some embodiments, the VEGF binding domain is a “VEGF receptor domain,” which describes a region of a polypeptide comprising one or more VEGF-binding regions of a VEGF receptor (e.g., VEGFR1 or VEGFR2). Exemplary VEGF receptor domains are described in Section 5.3.2.

In some embodiments, the VEGF binding domain is an anti-VEGF antibody (or fragment, portion, or component thereof). Exemplary anti-VEGF antibodies and antibody fragments are described in Section 5.3.3.

### 5.3.1. VEGF

Vascular endothelial growth factor (VEGF) belongs to the PDGF supergene family characterized by eight conserved cysteines and functions as a homodimer structure. The VEGF family of proteins includes VEGF-A, VEGF-B, VEGF-C, VEGF-D, and placental growth factor (PlGF) in mammals. VEGF-A is the most extensively studied of the VEGF family, and VEGF-A is generally referred to as simply “VEGF” for convenience. As used herein, the terms “vascular endothelial growth factor” and “VEGF” refer to mammalian VEGF-A, unless otherwise indicated.

VEGF-A regulates angiogenesis and vascular permeability by activating two receptors, VEGFR1 (also referred to as Flt-1) and VEGFR2 (also referred to as Flk1 or KDR). Thus, targeting VEGF-A is understood to reduce pathological angiogenesis in certain diseases, including but not limited to angiogenic eye disorders.

Exemplary amino acid sequences of VEGF (murine and human) are provided below:

Murine VEGF
(SEQ ID NO: 38)
MTDRQTDTAPSPSAHLLAGGLPTVDAAASREEPKPAPGGGVEGVGARGIA
RKLFVQLLGSSRSVVAVVCAAGDKPIGAGRSASSGLEKPGPEKRGEEEKE
EERGPQWALGSQEPSSWTGEAAVCADSAPAARAPQAPARASVPEGRGARQ
GAQESGLPRSPSRRGSASRAGPGRASETMNFLLSWHWTLALLLYLHHAKW
SQAAPTTEGEQKSHEVIKFMDVYQRSYCRPIETLVDIFQEYPDEIEYIFK
PSCVPLMRCAGCCNDEALECVPTSESNITMQIMRIKPHQSQHIGEMSFLQ
HSRCECRPKKDRTKPEKKSVRGKGKGQKRKRKKSRFKSWSVHCEPCSERR
KHLFVQDPQTCKCSCKNTDSRCKARQLELNERTCRCDKPRR
Human VEGF
(SEQ ID NO: 39)
MTDRQTDTAPSPSYHLLPGRRRTVDAAASRGQGPEPAPGGGVEGVGARGV
ALKLFVQLLGCSRFGGAVVRAGEAEPSGAARSASSGREEPQPEEGEEEEE
KEEERGPQWRLGARKPGSWTGEAAVCADSAPAARAPQALARASGRGGRVA
RRGAEESGPPHSPSRRGSASRAGPGRASETMNFLLSWVHWSLALLLYLHH
AKWSQAAPMAEGGGQNHHEVVKFMDVYQRSYCHPIETLVDIFQEYPDEIE
YIFKPSCVPLMRCGGCCNDEGLECVPTEESNITMQIMRIKPHQGQHIGEM
SFLQHNKCECRPKKDRARQEKKSVRGKGKGQKRKRKKSRYKSWSVPCGPC
SERRKHLFVQDPQTCKCSCKNTDSRCKARQLELNERTCRCDKPRR

In some embodiments, the VEGF binding domain of a VEGF antagonist of the disclosure specifically binds to murine VEGF. In some embodiments, the VEGF binding domain of a VEGF antagonist of the disclosure specifically binds to human VEGF.

### 5.3.2. VEGF Receptor Domains

In certain aspects, the VEGF binding domain included in a VEGF antagonist of the disclosure comprises one or more VEGF binding portions of a VEGF receptor that specifically bind to VEGF. Such a VEGF biding domain is sometimes referred to herein as a “VEGF receptor domain” purely for convenience. The term “VEGF receptor domain” includes a VEGF binding domain comprising one, two, or more VEGF binding portions of a VEGF receptor.

VEGF receptors VEGFR1 (Flt1) and VEGFR2 (FLK1 or KDR) bind to VEGF and are understood to mediate proangiogenic signaling in vascular endothelial cells via tyrosine kinase pathway. VEGFR1 has seven Ig-like domains, of which Ig-like domains two and three are understood to mediate VEGF binding (Wiesmann et al., 1997, Cell 91:695-704). Full length human VEGFR1 (Uniprot Accession No. P17948-1) and has the following canonical amino acid sequence:

(SEQ ID NO: 40)
MVSYWDTGVLLCALLSCLLLTGSSSGSKLKDPELSLKGTQHIMQAGQTLH
LQCRGEAAHKWSLPEMVSKESERLSITKSACGRNGKQFCSTLTLNTAQAN
HTGFYSCKYLAVPTSKKKETESAIYIFISDTGRPFVEMYSEIPEIIHMTE
GRELVIPCRVTSPNITVTLKKFPLDTLIPDGKRIIWDSRKGFIISNATYK
EIGLLTCEATVNGHLYKTNYLTHRQTNTIIDVQISTPRPVKLLRGHTLVL
NCTATTPLNTRVQMTWSYPDEKNKRASVRRRIDQSNSHANIFYSVLTIDK
MQNKDKGLYTCRVRSGPSFKSVNTSVHIYDKAFITVKHRKQQVLETVAGK
RSYRLSMKVKAFPSPEVVWLKDGLPATEKSARYLTRGYSLIIKDVTEEDA
GNYTILLSIKQSNVFKNLTATLIVNVKPQIYEKAVSSFPDPALYPLGSRQ
ILTCTAYGIPQPTIKWFWHPCNHNHSEARCDFCSNNEESFILDADSNMGN
RIESITQRMAIIEGKNKMASTLVVADSRISGIYICIASNKVGTVGRNISF
YITDVPNGFHVNLEKMPTEGEDLKLSCTVNKFLYRDVTWILLRTVNNRTM
HYSISKQKMAITKEHSITLNLTIMNVSLQDSGTYACRARNVYTGEEILQK
KEITIRDQEAPYLLRNLSDHTVAISSSTTLDCHANGVPEPQITWFKNNHK
IQQEPGIILGPGSSTLFIERVTEEDEGVYHCKATNQKGSVESSAYLTVQG
TSDKSNLELITLTCTCVAATLFWLLLTLFIRKMKRSSSEIKTDYLSIIMD
PDEVPLDEQCERLPYDASKWEFARERLKLGKSLGRGAFGKVVQASAFGIK
KSPTCRTVAVKMLKEGATASEYKALMTELKILTHIGHHLNVVNLLGACTK
QGGPLMVIVEYCKYGNLSNYLKSKRDLFFLNKDAALHMEPKKEKMEPGLE
QGKKPRLDSVTSSESFASSGFQEDKSLSDVEEEEDSDGFYKEPITMEDLI
SYSFQVARGMEFLSSRKCIHRDLAARNILLSENNVVKICDFGLARDIYKN
PDYVRKGDTRLPLKWMAPESIFDKIYSTKSDVWSYGVLLWEIFSLGGSPY
PGVQMDEDFCSRLREGMRMRAPEYSTPEIYQIMLDCWHRDPKERPRFAEL
VEKLGDLLQANVQQDGKDYIPINAILTGNSGFTYSTPAFSEDFFKESISA
PKFNSGSSDDVRYVNAFKFMSLERIKTFEELLPNATSMFDDYQGDSSTLL
ASPMLKRFTWTDSKPKASLKIDLRVTSKSKESGLSDVSRPSFCHSSCGHV
SEGKRRFTYDHAELERKIACCSPPPDYNSVVLYSTPPI

An amino acid sequence of the second Ig-like domain of human VEGFR1 is presented below (amino acids 151-214 of human VEGFR1):

(SEQ ID NO: 13)
GRELVIPCRVTSPNITVTLKKFPLDTLIPDGKRIIWDSRKGFIISNATYK
EIGLLTCEATVNGH

An additional amino acid sequence of the second Ig-like domain of human VEGFR1 is presented below (amino acids 128-232 of human VEGFR1):

(SEQ ID NO: 14)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IID

In some embodiments, the VEGF binding domain comprises a VEGF-binding portion of VEGFR1. In some embodiments, the VEGF binding domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO: 13. In some embodiments, the VEGF binding domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO: 14. Such a sequence is sometimes described herein as an “Ig-like domain 2” of VEGFR1, which term describes a region of a polypeptide having at least 90% sequence identity to SEQ ID NO: 13 or SEQ ID NO: 14.

Similar to VEGFR1, VEGFR2 has seven Ig-like domains, of which the second and third Ig-like domains are understood to mediate VEGF binding (Leppanen et al., 2010. PNAS 107:2425-2430). Full length human VEGFR2 (Uniprot Accession No. P35968-1) has the following canonical amino acid sequence:

(SEQ ID NO: 41)
MQSKVLLAVALWLCVETRAASVGLPSVSLDLPRLSIQKDILTIKANTTLQ
ITCRGQRDLDWLWPNNQSGSEQRVEVTECSDGLFCKTLTIPKVIGNDTGA
YKCFYRETDLASVIYVYVQDYRSPFIASVSDQHGVVYITENKNKTVVIPC
LGSISNLNVSLCARYPEKRFVPDGNRISWDSKKGFTIPSYMISYAGMVFC
EAKINDESYQSIMYIVVVVGYRIYDVVLSPSHGIELSVGEKLVLNCTART
ELNVGIDFNWEYPSSKHQHKKLVNRDLKTQSGSEMKKFLSTLTIDGVTRS
DQGLYTCAASSGLMTKKNSTFVRVHEKPFVAFGSGMESLVEATVGERVRI
PAKYLGYPPPEIKWYKNGIPLESNHTIKAGHVLTIMEVSERDTGNYTVIL
TNPISKEKQSHVVSLVVYVPPQIGEKSLISPVDSYQYGTTQTLTCTVYAI
PPPHHIHWYWQLEEECANEPSQAVSVTNPYPCEEWRSVEDFQGGNKIEVN
KNQFALIEGKNKTVSTLVIQAANVSALYKCEAVNKVGRGERVISFHVTRG
PEITLQPDMQPTEQESVSLWCTADRSTFENLTWYKLGPQPLPIHVGELPT
PVCKNLDTLWKLNATMFSNSTNDILIMELKNASLQDQGDYVCLAQDRKTK
KRHCVVRQLTVLERVAPTITGNLENQTTSIGESIEVSCTASGNPPPQIMW
FKDNETLVEDSGIVLKDGNRNLTIRRVRKEDEGLYTCQACSVLGCAKVEA
FFIIEGAQEKTNLEIIILVGTAVIAMFFWLLLVIILRTVKRANGGELKTG
YLSIVMDPDELPLDEHCERLPYDASKWEFPRDRLKLGKPLGRGAFGQVIE
ADAFGIDKTATCRTVAVKMLKEGATHSEHRALMSELKILIHIGHHLNVVN
LLGACTKPGGPLMVIVEFCKFGNLSTYLRSKRNEFVPYKTKGARFRQGKD
YVGAIPVDLKRRLDSITSSQSSASSGFVEEKSLSDVEEEEAPEDLYKDFL
TLEHLICYSFQVAKGMEFLASRKCIHRDLAARNILLSEKNVVKICDFGLA
RDIYKDPDYVRKGDARLPLKWMAPETIFDRVYTIQSDVWSFGVLLWEIFS
LGASPYPGVKIDEEFCRRLKEGTRMRAPDYTTPEMYQTMLDCWHGEPSQR
PTFSELVEHLGNLLQANAQQDGKDYIVLPISETLSMEEDSGLSLPTSPVS
CMEEEEVCDPKFHYDNTAGISQYLQNSKRKSRPVSVKTFEDIPLEEPEVK
VIPDDNQTDSGMVLASEELKTLEDRTKLSPSFGGMVPSKSRESVASEGSN
QTSGYQSGYHSDDTDTTVYSSEEAELLKLIEIGVQTGSTAQILQPDSGTT
LSSPPV

An amino acid sequence of the third Ig-like domain of VEGFR2 is presented below (amino acids 226-321 of human VEGFR2):

(SEQ ID NO: 15)
VVLSPSHGIELSVGEKLVLNCTARTELNVGIDFNWEYPSSKHQHKKLVNR
DLKTQSGSEMKKFLSTLTIDGVTRSDQGLYTCAASSGLMTKKNSTFVRVH
EK

An additional amino acid sequence of the third Ig-like domain of VEGFR2 is presented below (amino acids 226-321 of human VEGFR2):

(SEQ ID NO: 50)
VVLSPSHGIELSVGEKLVLNCTARTELNVGIDFNWEYPSSKHQHKKLVNR
DLKTQSGSEMKKFLSTLTIDGVTRSDQGLYTCAASSGLMTKKNST

In some embodiments, the VEGF binding domain comprises a VEGF-binding portion of VEGFR2. In some embodiments, the VEGF binding domain comprises an amino acid sequence having at least 90%, at least 91%, at least 92%, at least 93%, at least 94%, at least 95%, at least 96%, at least 97%, at least 98%, at least 99%, or 100% sequence identity to the third Ig-like domain of human VEGR2 (SEQ ID NO:15). Such a sequence is described in some cases herein as an “Ig-like domain 3” of VEGFR2, which term describes a region of a polypeptide having at least 90% sequence identity to SEQ ID NO: 15. In some embodiments, the VEGF binding domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:50.

In some embodiments, the VEGF binding domain comprises an Ig-like domain 2 of VEGFR1 and an Ig-like domain 3 of VEGFR2. An example sequence of a VEGF receptor binding domain comprising an Ig-like domain 2 of VEGFR1 (bolded) and an Ig-like domain 3 of VEGFR2 (italicized) is provided below:

(SEQ ID NO: 16)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEK

In some embodiments, the VEGF binding domain comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 16. In some embodiments, the VEGF binding domain comprises an amino acid sequence having at least 90% sequence identity to SEQ ID NO: 16. In some embodiments, the VEGF binding domain comprises an amino acid sequence having at least 91% sequence identity to SEQ ID NO: 16. In some embodiments, the VEGF binding domain comprises an amino acid sequence having at least 92% sequence identity to SEQ ID NO: 16. In some embodiments, the VEGF binding domain comprises an amino acid sequence having at least 93% sequence identity to SEQ ID NO: 16. In some embodiments, the VEGF binding domain comprises an amino acid sequence having at least 94% sequence identity to SEQ ID NO: 16. In some embodiments, the VEGF binding domain comprises an amino acid sequence having at least 95% sequence identity to SEQ ID NO:16. In some embodiments, the VEGF binding domain comprises an amino acid sequence having at least 96% sequence identity to SEQ ID NO: 16. In some embodiments, the VEGF binding domain comprises an amino acid sequence having at least 97% sequence identity to SEQ ID NO: 16. In some embodiments, the VEGF binding domain comprises an amino acid sequence having at least 98% sequence identity to SEQ ID NO: 16. In some embodiments, the VEGF binding domain comprises an amino acid sequence having at least 98.5% sequence identity to SEQ ID NO: 16. In some embodiments, the VEGF binding domain comprises an amino acid sequence having at least 99% sequence identity to SEQ ID NO:16. In some embodiments, the VEGF binding domain comprises an amino acid sequence having at least 99.5% sequence identity to SEQ ID NO: 16. In some embodiments, the VEGF binding domain comprises the amino acid sequence of SEQ ID NO: 16.

In some embodiments, the VEGF binding domain comprises an amino acid sequence having at most one, two, three, four, five, six, seven, eight, nine, or ten amino acid differences (e.g., substitutions, deletions, or additions) relative to SEQ ID NO:16. In some embodiments, the VEGF binding domain comprises an amino acid sequence having at most ten amino acid differences (e.g., substitutions, deletions, or additions) relative to SEQ ID NO:16. In some embodiments, the VEGF binding domain comprises an amino acid sequence having at most nine amino acid differences (e.g., substitutions, deletions, or additions) relative to SEQ ID NO: 16. In some embodiments, the VEGF binding domain comprises an amino acid sequence having at most eight amino acid differences (e.g., substitutions, deletions, or additions) relative to SEQ ID NO: 16. In some embodiments, the VEGF binding domain comprises an amino acid sequence having at most seven amino acid differences (e.g., substitutions, deletions, or additions) relative to SEQ ID NO:16. In some embodiments, the VEGF binding domain comprises an amino acid sequence having at most six amino acid differences (e.g., substitutions, deletions, or additions) relative to SEQ ID NO: 16. In some embodiments, the VEGF binding domain comprises an amino acid sequence having at most five amino acid differences (e.g., substitutions, deletions, or additions) relative to SEQ ID NO: 16. In some embodiments, the VEGF binding domain comprises an amino acid sequence having at most four amino acid differences (e.g., substitutions, deletions, or additions) relative to SEQ ID NO: 16. In some embodiments, the VEGF binding domain comprises an amino acid sequence having at most three amino acid differences (e.g., substitutions, deletions, or additions) relative to SEQ ID NO: 16. In some embodiments, the VEGF binding domain comprises an amino acid sequence having at most two amino acid differences (e.g., substitutions, deletions, or additions) relative to SEQ ID NO:16. In some embodiments, the VEGF binding domain comprises an amino acid sequence having only one amino acid difference (e.g., substitution, deletion, or addition) relative to SEQ ID NO: 16.

### 5.3.3. Anti-VEGF Antibodies and Fragments

In some aspects, the VEGF binding domain included in a VEGF antagonist of the disclosure can be any type of antibody fragment that specifically binds to VEGF or an epitope thereof. Antibody fragments include, but are not limited to, VH (or VH fragments), VL (or VL) fragments, VHH antibodies (nanobodies), Fab fragments, F(ab′)2 fragments, scFv fragments, Fv fragments, minibodies, diabodies, triabodies, and tetrabodies. Where an antigen binding domain includes two separate polypeptide chains (e.g., a Fab), a first portion (e.g., comprising VH) can be operably linked to the N-terminus of an Fc domain and a second polypeptide chain (e.g., comprising VL) can be included as a separate polypeptide chain capable of associating with a VH.

Exemplary VEGF binder sequences that can be incorporated into a VEGF binding domain of the disclosure are identified in Table T-1.

TABLE T-1
Exemplary Anti-VEGF Antibody Sequences

| Antibody Name and/or | |
|---|---|
| Binding Sequence | |
| References | Sequence |
| VH: SEQ ID NO: 3 of | VH: |
| U.S. Pat. No. | QVQLVQSGAEVKKPGASVKVSCKASGGTFSSYAISWVRQAPGQGLEWMGGFDPEDGE |
| 9,421,256 | TIYAQKFQGRVTMTEDTSTDTAYMELSSLRSEDTAVYYCATGRSMVRGVIIPFNGMD |
| | VWGQGTTVTVSS (SEQ ID NO: 42) |
| VL: SEQ ID NO: 4 of | VL: |
| U.S. Pat. No. | DIRMTQSPSSLSASVGDRVTITCRASQSISSYLNWYQQKPGKAPKLLIYAASSLQSG |
| 9,421,256 | VPSRFSGSGSGTDFTLTISSLQPEDFATYYCQQSYSTPLTFGGGTKVEIK (SEQ |
| | ID NO: 43) |
| VH: SEQ ID NO: 11 of | VH: |
| U.S. Pat. No. | EVQLVESGGGLVQPGGSLRLSCAASGFTISDYWIHWVRQAPGKGLEWVAGITPAGGY |
| 11,891,437 | TRYADSVKGRFTISADTSKNTAYLQMRSLRAEDTAVYYCARFVFFLPYAMDYWGQGT |
| | LVTVSS (SEQ ID NO: 44) |
| VL: SEQ ID NO: 12 of | VL: |
| U.S. Pat. No. | DIQMTQSPSSLSASVGDRVTITCRASQDVSTAVAWYQQKPGKAPKLLIYSASFLYSG |
| 11,891,437 | VPSRFSGSGSGTDFTLTISSLQPEDAATYYCQQGYGAPFTFGQGTKVEIK (SEQ |
| | ID NO: 45) |
| VH: SEQ ID NO: 33 of | VH: |
| U.S. Pat. No. | EEQLVEEGGGLVQPGESLELSCAASGFEISDYWIHWVRQEPGEGLEWVAGITPAGGY |
| 11,891,437 | EYYADSVEGRFTISADTSENTAYLQMNELRAEDTAVYYCARFVFFLPYAMDYWGQGE |
| | LVTVSS (SEQ ID NO: 46) |
| VL: SEQ ID NO: 38 of | VL: |
| U.S. Pat. No. | DIQMTQSPSSLSASVGDRVTITCRASQDVSTAVAWYQQKPGKAPKLLIYSASFLYSG |
| 11,891,437 | VPSRFSGSGSGTDFTLTISSLQPEDFATYYCQQGYGNPFTFGQGTKVEIK (SEQ |
| | ID NO: 47) |
| VH: SEQ ID NO: 33 of | VH: |
| U.S. Pat. No. | EEQLVEEGGGLVQPGESLELSCAASGFEISDYWIHWVRQEPGEGLEWVAGITPAGGY |
| 11,891,437 | EYYADSVEGRFTISADTSENTAYLQMNELRAEDTAVYYCARFVFFLPYAMDYWGQGE |
| | LVTVSS (SEQ ID NO: 46) |
| VL: SEQ ID NO: 34 of | VL: |
| U.S. Pat. No. | DIQMTQSPESLSASVGDEVTITCRASQDVSTAVAWYQQKPGKAPKLLIYSASFLYSG |
| 11,891,437 | VPSRFSGSGSGTDFTLTISSLQPEDAATYYCQQGYGAPFTFGQGTKVEIK (SEQ |
| | ID NO: 48) |
| VH: SEQ ID NO: 33 of | VH: |
| U.S. Pat. No. | EEQLVEEGGGLVQPGESLELSCAASGFEISDYWIHWVRQEPGEGLEWVAGITPAGGY |
| 11,891,437 | EYYADSVEGRFTISADTSENTAYLQMNELRAEDTAVYYCARFVFFLPYAMDYWGQGE |
| | LVTVSS (SEQ ID NO: 46) |
| VL: SEQ ID NO: 35 of | VL: |
| U.S. Pat. No. | DIQMTQSPSSLSASVGDEVTITCRASQDVSTAVAWYQQKPGEAPKLLIYSASFLYSG |
| 11,891,437 | VPSRFSGSGSGTDFTLTIESLQPEDAATYYCQQGYGAPFTFGQGTKVEIK (SEQ |
| | ID NO: 49) |
| VH: SEQ ID NO: 33 of | VH: |
| U.S. Pat. No. | EEQLVEEGGGLVQPGESLELSCAASGFEISDYWIHWVRQEPGEGLEWVAGITPAGGY |
| 11,891,437 | EYYADSVEGRFTISADTSENTAYLQMNELRAEDTAVYYCARFVFFLPYAMDYWGQGE |
| | LVTVSS (SEQ ID NO: 46) |
| VL: SEQ ID NO: 36 of | VL: |
| U.S. Pat. No. | DIQMTQSPESLSASVGDEVTITCRASQDVSTAVAWYQQKPGEAPKLLIYSASFLYSG |
| 11,891,437 | VPSRFSGSGSGTDFTLTISSLQPEDAATYYCQQGYGAPFTFGQGTKVEIK (SEQ |
| | ID NO: 59) |
| VH: SEQ ID NO: 33 of | VH: |
| U.S. Pat. No. | EEQLVEEGGGLVQPGESLELSCAASGFEISDYWIHWVRQEPGEGLEWVAGITPAGGY |
| 11,891,437 | EYYADSVEGRFTISADTSENTAYLQMNELRAEDTAVYYCARFVFFLPYAMDYWGQGE |
| | LVTVSS (SEQ ID NO: 46) |
| VL: SEQ ID NO: 12 of | VL: |
| U.S. Pat. No. | DIQMTQSPSSLSASVGDRVTITCRASQDVSTAVAWYQQKPGKAPKLLIYSASFLYSG |
| 11,891,437 | VPSRFSGSGSGTDFTLTISSLQPEDAATYYCQQGYGAPFTFGQGTKVEIK (SEQ |
| | ID NO: 45) |
| VH: SEQ ID NO: 51 of | VH: |
| U.S. Pat. No. | EEQLVEEGGGLVQPGESLRLSCAASGFEISDYWIHWVRQEPGEGLEWVAGITPAGGY |
| 11,891,437 | EYYADSVEGRFTISADTSENTAYLQMNELRAEDTAVYYCARFVFFLPYAMDYWGQGE |
| | LVTVSS (SEQ ID NO: 60) |
| VL: SEQ ID NO: 38 of | VL: |
| U.S. Pat. No. | DIQMTQSPSSLSASVGDRVTITCRASQDVSTAVAWYQQKPGKAPKLLIYSASFLYSG |
| 11,891,437 | VPSRFSGSGSGTDFTLTISSLQPEDFATYYCQQGYGNPFTFGQGTKVEIK (SEQ |
| | ID NO: 47) |
| VH: SEQ ID NO: 51 of | VH: |
| U.S. Pat. No. | EEQLVEEGGGLVQPGESLRLSCAASGFEISDYWIHWVRQEPGEGLEWVAGITPAGGY |
| 11,891,437 | EYYADSVEGRFTISADTSENTAYLQMNELRAEDTAVYYCARFVFFLPYAMDYWGQGE |
| | LVTVSS (SEQ ID NO: 60) |
| VL: SEQ ID NO: 35 of | VL: |
| U.S. Pat. No. | DIQMTQSPSSLSASVGDEVTITCRASQDVSTAVAWYQQKPGEAPKLLIYSASFLYSG |
| 11,891,437 | VPSRFSGSGSGTDFTLTIESLQPEDAATYYCQQGYGAPFTFGQGTKVEIK (SEQ |
| | ID NO: 49) |
| VH: SEQ ID NO: 51 of | VH: |
| U.S. Pat. No. | EEQLVEEGGGLVQPGESLRLSCAASGFEISDYWIHWVRQEPGEGLEWVAGITPAGGY |
| 11,891,437 | EYYADSVEGRFTISADTSENTAYLQMNELRAEDTAVYYCARFVFFLPYAMDYWGQGE |
| | LVTVSS (SEQ ID NO: 60) |
| VL: SEQ ID NO: 37 of | VL: |
| U.S. Pat. No. | DIQMTQSPSSLSASVGDEVTITCRASQDVSTAVAWYQQKPGKAPKLLIYSASFLYSG |
| 11,891,437 | VPSRFSGSGSGTDFTLTISSLQPEDAATYYCQQGYGAPFTFGQGTKVEIK (SEQ |
| | ID NO: 61) |
| VH: SEQ ID NO: 51 of | VH: |
| U.S. Pat. No. | EEQLVEEGGGLVQPGESLRLSCAASGFEISDYWIHWVRQEPGEGLEWVAGITPAGGY |
| 11,891,437 | EYYADSVEGRFTISADTSENTAYLQMNELRAEDTAVYYCARFVFFLPYAMDYWGQGE |
| | LVTVSS (SEQ ID NO: 60) |
| VL: SEQ ID NO: 12 of | VL: |
| U.S. Pat. No. | DIQMTQSPSSLSASVGDRVTITCRASQDVSTAVAWYQQKPGKAPKLLIYSASFLYSG |
| 11,891,437 | VPSRFSGSGSGTDFTLTISSLQPEDAATYYCQQGYGAPFTFGQGTKVEIK (SEQ |
| | ID NO: 45) |
| VH: SEQ ID NO: 42 of | VH: |
| U.S. Pat. No. | EVQLVESGGGLVQPGGSLRLSCAASGFTISDYWIHWVRQAPGKGLEWVAGITPAGGY |
| 11,891,437 | TYYADSVKGRFTISADTSKNTAYLQMNSLRAEDTAVYYCARFVFFLPYAMDYWGQGT |
| | LVTVSS (SEQ ID NO: 62) |
| VL: SEQ ID NO: 38 of | VL: |
| U.S. Pat. No. | DIQMTQSPSSLSASVGDRVTITCRASQDVSTAVAWYQQKPGKAPKLLIYSASFLYSG |
| 11,891,437 | VPSRFSGSGSGTDFTLTISSLQPEDFATYYCQQGYGNPFTFGQGTKVEIK (SEQ |
| | ID NO: 47) |
| VH: SEQ ID NO: 42 of | VH: |
| U.S. Pat. No. | EVQLVESGGGLVQPGGSLRLSCAASGFTISDYWIHWVRQAPGKGLEWVAGITPAGGY |
| 11,891,437 | TYYADSVKGRFTISADTSKNTAYLQMNSLRAEDTAVYYCARFVFFLPYAMDYWGQGT |
| | LVTVSS (SEQ ID NO: 62) |
| VL: SEQ ID NO: 41 of | VL: |
| U.S. Pat. No. | DIQMTQSPSSLSASVGDRVTITCRASQDVSTAVAWYQQKPGKAPKLLIYSASFLYSG |
| 11,891,437 | VPSRFSGSGSGTDFTLTISSLOPEDFATYYCQQGYGAPFTFGQGTKVEIK (SEQ |
| | ID NO: 63) |
| VH: SEQ ID NO: 42 of | VH: |
| U.S. Pat. No. | EVQLVESGGGLVQPGGSLRLSCAASGFTISDYWIHWVRQAPGKGLEWVAGITPAGGY |
| 11,891,437 | TYYADSVKGRFTISADTSKNTAYLQMNSLRAEDTAVYYCARFVFFLPYAMDYWGQGT |
| | LVTVSS (SEQ ID NO: 62) |
| VL: SEQ ID NO: 12 of | VL: |
| U.S. Pat. No. | DIQMTQSPSSLSASVGDRVTITCRASQDVSTAVAWYQQKPGKAPKLLIYSASFLYSG |
| 11,891,437 | VPSRFSGSGSGTDFTLTISSLQPEDAATYYCQQGYGAPFTFGQGTKVEIK (SEQ |
| | ID NO: 45) |
| VH: SEQ ID NO: 40 of | VH: |
| U.S. Pat. No. | EVQLVESGGGLVQPGGSLRLSCAASGFTISDYWIHWVRQEPGKGLEWVAGITPAGGY |
| 11,891,437 | EYYADSVKGRFTISADTSKNTAYLQMNSLRAEDTAVYYCARFVFFLPYAMDYWGQGT |
| | LVTVSS (SEQ ID NO: 64) |
| VL: SEQ ID NO: 12 of | VL: |
| U.S. Pat. No. | DIQMTQSPSSLSASVGDRVTITCRASQDVSTAVAWYQQKPGKAPKLLIYSASFLYSG |
| 11,891,437 | VPSRFSGSGSGTDFTLTISSLQPEDAATYYCQQGYGAPFTFGQGTKVEIK (SEQ |
| | ID NO: 45) |
| Bevacizumab | VH: |
| VH: SEQ ID NO: 7 of | EVQLVESGGGLVQPGGSLRLSCAASGYTFTNYGMNWVRQAPGKGLEWVGWINTYTGE |
| U.S. Application No. | PTYAADFKRRFTFSLDTSKSTAYLQMNSLRAEDTAVYYCAKYPHYYGSSHWYFDVWG |
| 2023/0399391 | QGTLVTVSS (SEQ ID NO: 65) |
| VL: SEQ ID NO: 8 of | VL: |
| U.S. Application No. | DIQMTQSPSSLSASVGDRVTITCSASQDISNYLNWYQQKPGKAPKVLIYFTSSLHSG |
| 2023/0399391 | VPSRFSGSGSGTDFTLTISSLOPEDFATYYCQQYSTVPWTFGQGTKVEIK (SEQ |
| | ID NO: 66) |
| Ranibizumab | VH: |
| VH: SEQ ID NO: 15 of | EVQLVESGGGLVQPGGSLRLSCAASGYDFTHYGMNWVRQAPGKGLEWVGWINTYTGE |
| U.S. Application No. | PTYAADFKRRFTFSLDTSKSTAYLQMNSLRAEDTAVYYCAKYPYYYGTSHWYFDVWG |
| 2023/0399391 | QGTLVTVSS (SEQ ID NO: 67) |
| VL: SEQ ID NO: 16 of | VL: |
| U.S. Application No. | DIQLTQSPSSLSASVGDRVTITCSASQDISNYLNWYQQKPGKAPKVLIYFTSSLHSG |
| 2023/0399391 | VPSRFSGSGSGTDFTLTISSLQPEDFATYYCQQYSTVPWTFGQGTKVEIK (SEQ |
| | ID NO: 68) |
| HuMab G6-31 | VH: |
| VH: SEQ ID NO: 23 of | EVQLVESGGGLVQPGGSLRLSCAASGFTISDYWIHWVRQAPGKGLEWVAGITPAGGY |
| U.S. Application No. | TYYADSVKGRFTISADTSKNTAYLQMNSLRAEDTAVYYCARFVFFLPYAMDYWGQGT |
| 2023/0399391 | LVTVSS (SEQ ID NO: 62) |
| VL: SEQ ID NO: 24 of | VL: |
| U.S. Application No. | DIQMTQSPSSLSASVGDRVTITCRASQDVSTAVAWYQQKPGKAPKLLIYSASFLYSG |
| 2023/0399391 | VPSRFSGSGSGTDFTLTISSLQPEDFATYYCQQGYGNPFTFGQGTKVEIK (SEQ |
| | ID NO: 47) |
| B20-4.1 | VH: |
| VH: SEQ ID NO: 100 of | EVQLVESGGGLVQPGGSLRLSCAASGFSINGSWIFWVRQAPGKGLEWVGAIWPFGGY |
| U.S. Application No. | THYADSVKGRFTISADTSKNTAYLQMNSLRAEDTAVYYCARWGHSTSPWAMDYWGQG |
| 2023/0399391 | TLV (SEQ ID NO: 69) |
| VL: SEQ ID NO: 101 of | VL: |
| U.S. Application No. | DIQMTQSPSSLSASVGDRVTITCRASQVIRRSLAWYQQKPGKAPKLLIYAASNLASG |
| 2023/0399391 | VPSRFSGSGSGTDFTLTISSLQPEDFATYYCQQSNTSPLTFGQGTKVEIKR (SEQ |
| | ID NO: 70) |

### 5.3.3.1. Fab

Fab domains were traditionally produced by proteolytic cleavage of immunoglobulin molecules using enzymes such as papain. The Fab domains can comprise constant domain and variable region sequences from any suitable species, and thus can be murine, chimeric, human or humanized.

Fab domains typically comprise a CH1 domain attached to a VH domain which pairs with a CL domain attached to a VL domain. In a wild-type immunoglobulin, the VH domain is paired with the VL domain to constitute the Fv region, and the CH1 domain is paired with the CL domain to further stabilize the binding site. A disulfide bond between the two constant domains can further stabilize the Fab domain.

For the VEGF antagonists of the disclosure, particularly when the light chains of the target binding domains are not common or universal light chains, it is advantageous to use Fab heterodimerization strategies to permit the correct association of Fab domains belonging to the same target binding domain and minimize aberrant pairing of Fab domains belonging to different target binding domains. For example, the Fab heterodimerization strategies shown in Table H below can be used:

TABLE H
Fab Heterodimerization Strategies

| STRATEGY | VH | CH1 | VL | CL | REFERENCE |
|---|---|---|---|---|---|
| CrossMabCH | WT | CL domain | WT | CH1 domain | Schaefer et al., 2011, |
| 1-CL | | | | | Cancer Cell 2011; |
| | | | | | 20: 472-86; |
| | | | | | PMID: 22014573. |
| orthogonal Fab | 39K, 62E | H172A, F174G | 1R, 38D, (36F) | L135Y, S176W | Lewis et al., 2014, Nat |
| VHVRD1CH1 CRD2 - | | | | | Biotechnol 32: 191-8 |
| VLVRD1CλCRD2 | | | | | |
| orthogonal Fab | 39Y | WT | 38R | WT | Lewis et al., 2014, Nat |
| VHVRD2CH1 wt - | | | | | Biotechnol 32: 191-8 |
| VLVRD2Cλwt | | | | | |
| TCR CαCβ | 39K | TCR Cα | 38D | TCR Cβ | Wu et al., 2015, MAbs |
| | | | | | 7: 364-76 |
| CR3 | WT | T192E | WT | N137K, S114A | Golay at al., 2016, J |
| | | | | | Immunol 196: 3199- |
| | | | | | 211. |
| MUT4 | WT | L143Q, S188V | WT | V133T, S176V | Golay at al., 2016, J |
| | | | | | Immunol 196: 3199- |
| | | | | | 211. |
| DuetMab | WT | F126C | WT | S121C | Mazor et al., 2015, |
| | | | | | MAbs 7: 377-89; Mazor |
| | | | | | et al., 2015, MAbs |
| | | | | | 7: 461-669. |
| Domain | WT | CH3 + knob or | WT | CH3 + hole or | Wozniak-Knopp et al., 2018, |
| exchanged | | hole mutation | | knob mutation | PLoSONE13(4): e0195442 |

Accordingly, in certain embodiments, correct association between the two polypeptides of a Fab is promoted by exchanging the VL and VH domains of the Fab for each other or exchanging the CH1 and CL domains for each other, e.g., as described in WO 2009/080251.

Correct Fab pairing can also be promoted by introducing one or more amino acid modifications in the CH1 domain and one or more amino acid modifications in the CL domain of the Fab and/or one or more amino acid modifications in the VH domain and one or more amino acid modifications in the VL domain. The amino acids that are modified are typically part of the VH: VL and CH1: CL interface such that the Fab components preferentially pair with each other rather than with components of other Fabs.

In one embodiment, the one or more amino acid modifications are limited to the conserved framework residues of the variable (VH, VL) and constant (CH1, CL) domains as indicated by the Kabat numbering of residues. Almagro, 2008, Frontiers in Bioscience 13:1619-1633 provides a definition of the framework residues on the basis of Kabat, Chothia, and IMGT numbering schemes.

In one embodiment, the modifications introduced in the VH and CH1 and/or VL and CL domains are complementary to each other. Complementarity at the heavy and light chain interface can be achieved on the basis of steric and hydrophobic contacts, electrostatic/charge interactions or a combination of the variety of interactions. The complementarity between protein surfaces is broadly described in the literature in terms of lock and key fit, knob into hole, protrusion and cavity, donor and acceptor etc., all implying the nature of structural and chemical match between the two interacting surfaces.

In one embodiment, the one or more introduced modifications introduce a new hydrogen bond across the interface of the Fab components. In one embodiment, the one or more introduced modifications introduce a new salt bridge across the interface of the Fab components. Exemplary substitutions are described in WO 2014/150973 and WO 2014/082179, the contents of which are hereby incorporated by reference.

In some embodiments, the Fab domain comprises a 192E substitution in the CH1 domain and 114A and 137K substitutions in the CL domain, which introduces a salt-bridge between the CH1 and CL domains (see, e.g., Golay et al., 2016, J Immunol 196:3199-211).

In some embodiments, the Fab domain comprises a 143Q and 188V substitutions in the CH1 domain and 113T and 176V substitutions in the CL domain, which serves to swap hydrophobic and polar regions of contact between the CH1 and CL domain (see, e.g., Golay et al., 2016, J Immunol 196:3199-211).

In some embodiments, the Fab domain can comprise modifications in some or all of the VH, CH1, VL, CL domains to introduce orthogonal Fab interfaces which promote correct assembly of Fab domains (Lewis et al., 2014 Nature Biotechnology 32:191-198). In an embodiment, 39K, 62E modifications are introduced in the VH domain, H172A, F174G modifications are introduced in the CH1 domain, 1R, 38D, (36F) modifications are introduced in the VL domain, and L135Y, S176W modifications are introduced in the CL domain. In another embodiment, a 39Y modification is introduced in the VH domain and a 38R modification is introduced in the VL domain.

Fab domains can also be modified to replace the native CH1: CL disulfide bond with an engineered disulfide bond, thereby increasing the efficiency of Fab component pairing. For example, an engineered disulfide bond can be introduced by introducing a 126C in the CH1 domain and a 121 C in the CL domain (see, e.g., Mazor et al., 2015, MAbs 7:377-89).

Fab domains can also be modified by replacing the CH1 domain and CL domain with alternative domains that promote correct assembly. For example, Wu et al., 2015, MAbs 7:364-76, describes substituting the CH1 domain with the constant domain of the T cell receptor and substituting the CL domain with the b domain of the T cell receptor, and pairing these domain replacements with an additional charge-charge interaction between the VL and VH domains by introducing a 38D modification in the VL domain and a 39K modification in the VH domain.

In lieu of, or in addition to, the use of Fab heterodimerization strategies to promote correct VH-VL pairings, the VL of common light chain (also referred to as a universal light chain) can be used for each unique target binding domain in the VEGF antagonists of the disclosure. In various embodiments, employing a common light chain as described herein reduces the number of inappropriate species in the VEGF antagonists as compared to employing original cognate VLs. In various embodiments, the VL domains of target binding domains are identified from monospecific antibodies comprising a common light chain. In various embodiments, the VH regions of the target binding domains in the VEGF antagonists comprise human heavy chain variable gene segments that are rearranged in vivo within mouse B cells that have been previously engineered to express a limited human light chain repertoire, or a single human light chain, cognate with human heavy chains and, in response to exposure with an antigen of interest, generate an antibody repertoire containing a plurality of human VHs that are cognate with one or one of two possible human VLs, wherein the antibody repertoire specific for the antigen of interest. Common light chains are those derived from a rearranged human Vκ1-39Jκ5 sequence or a rearranged human Vκ3-20Jκ1 sequence, and include somatically mutated (e.g., affinity matured) versions. See, for example, U.S. Pat. No. 10,412,940.

5.3.3.2. scFv

Single chain Fv or “scFv” antibody fragments comprise the VH and VL domains of an antibody in a single polypeptide chain, are capable of being expressed as a single chain polypeptide, and retain the specificity of the intact antibodies from which they are derived. Generally, the scFv polypeptide further comprises a polypeptide linker between the VH and VL domain that enables the scFv to form the desired structure for target binding. Examples of linkers suitable for connecting the VH and VL chains of an scFv are described in Section 5.5.

Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The scFv can comprise VH and VL sequences from any suitable species, such as murine, human or humanized VH and VL sequences.

To create an scFv-encoding nucleic acid, the VH and VL-encoding DNA fragments are operably linked to another fragment encoding a linker, e.g., encoding any of the linkers described in Section 5.5 (typically a repeat of a sequence containing the amino acids glycine and serine, such as the amino acid sequence (GGGGS) (SEQ ID NO: 71)), such that the VH and VL sequences can be expressed as a contiguous single-chain protein, with the VL and VH regions joined by the flexible linker (see, e.g., Bird et al., 1988, Science 242:423-426; Huston et al., 1988, Proc. Natl. Acad. Sci. USA 85:5879-5883; McCafferty et al., 1990, Nature 348:552-554).

### 5.4. Multimerization Domains

The VEGF antagonists of the disclosure comprise a multimerization domain. In some embodiments, the multimerization domain comprises an Fc domain. A multimerization domain of the disclosure in some cases further comprises a tailpiece C-terminal to the Fc domain. Exemplary Fc domains are described further in Section 5.4.1, and subsections thereof. Exemplary tailpieces are described further in Section 5.4.2.

### 5.4.1. Fc Domains

In some embodiments, VEGF antagonists of the disclosure include at least one Fc domain, and typically comprise two Fc domains that associate to form an Fc region. In native antibodies, Fc regions comprise hinge regions at their N-termini. Throughout this disclosure, the reference to an Fc domain encompasses (but is not limited to) an Fc domain with a hinge domain at its N-terminus unless specified otherwise. Exemplary hinge domains are set forth in Section 5.6.

The Fc domains can be derived from any suitable species operably linked to a target binding domain or component thereof. In one embodiment the Fc domain is derived from a human Fc domain.

The Fc domains can be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domain is derived from IgG1, IgG2, IgG3 or IgG4. In one embodiment the Fc domain is derived from IgG1. In one embodiment the Fc domain is derived from IgG4.

Example Fc domain sequences are provided in Table F-1, below.

TABLE F-1
Fc Sequences

| | | SEQ |
|---|---|---|
| Fc | Sequence | ID NO |
| hIgG1 Fc | EPKSCDKTHTCPPCPAPELLGGPSVFLFPPKPKDTLMISRTPEVT | 1 |
| (amino acids | CVVVDVSHEDPEVKFNWYVDGVEVHNAKTKPREEQYNSTYRVVSV | |
| 99-330 of | LTVLHQDWLNGKEYKCKVSNKALPAPIEKTISKAKGQPREPQVYT | |
| UniprotKB | LPPSRDELTKNQVSLTCLVKGFYPSDIAVEWESNGQPENNYKTTP | |
| P01857-1) | PVLDSDGSFFLYSKLTVDKSRWQQGNVFSCSVMHEALHNHYTQKS | |
| | LSLSPGK | |
| hIgG2 Fc | ERKCCVECPPCPAPPVAGPSVFLFPPKPKDTLMISRTPEVTCVVV | 21 |
| (amino acids | DVSHEDPEVQFNWYVDGVEVHNAKTKPREEQFNSTFRVVSVLTVV | |
| 99-326 of | HQDWLNGKEYKCKVSNKGLPAPIEKTISKTKGQPREPQVYTLPPS | |
| UniprotKB | REEMTKNQVSLTCLVKGFYPSDISVEWESNGQPENNYKTTPPMLD | |
| P01859-1) | SDGSFFLYSKLTVDKSRWQQGNVFSCSVMHEALHNHYTQKSLSLS | |
| | PGK | |
| hIgG3 Fc | ELKTPLGDTTHTCPRCPEPKSCDTPPPCPRCPEPKSCDTPPPCPR | 22 |
| (amino acids | CPEPKSCDTPPPCPRCPAPELLGGPSVFLFPPKPKDTLMISRTPE | |
| 99-377 of | VTCVVVDVSHEDPEVQFKWYVDGVEVHNAKTKPREEQYNSTFRVV | |
| UniprotKB | SVLTVLHQDWLNGKEYKCKVSNKALPAPIEKTISKTKGQPREPQV | |
| P01860-1) | YTLPPSREEMTKNQVSLTCLVKGFYPSDIAVEWESSGQPENNYNT | |
| | TPPMLDSDGSFFLYSKLTVDKSRWQQGNIFSCSVMHEALHNRFTQ | |
| | KSLSLSPGK | |
| hIgG4 Fc | ESKYGPPCPSCPAPEFLGGPSVFLFPPKPKDTLMISRTPEVTCVV | 23 |
| (amino acids | VDVSQEDPEVQFNWYVDGVEVHNAKTKPREEQFNSTYRVVSVLTV | |
| 99-327 of | LHQDWLNGKEYKCKVSNKGLPSSIEKTISKAKGQPREPQVYTLPP | |
| UniprotKB | SQEEMTKNQVSLTCLVKGFYPSDIAVEWESNGQPENNYKTTPPVL | |
| P01861-1) | DSDGSFFLYSRLTVDKSRWQEGNVFSCSVMHEALHNHYTQKSLSL | |
| | SLGK | |
| hIgG4s Fc | ESKYGPPCPPCPAPPVAGPSVFLFPPKPKDTLMISRTPEVTCVVV | 24 |
| | DVSQEDPEVQFNWYVDGVEVHNAKTKPREEQFNSTYRVVSVLTVL | |
| | HQDWLNGKEYKCKVSNKGLPSSIEKTISKAKGQPREPQVYTLPPS | |
| | QEEMTKNQVSLTCLVKGFYPSDIAVEWESNGQPENNYKTTPPVLD | |
| | SDGSFFLYSRLTVDKSRWQEGNVFSCSVMHEALHNHYTQKSLSLS | |
| | LGK | |
| hIgG1 PVA Fc | EPKSCDKTHTCPPCPAPPVAGPSVFLFPPKPKDTLMISRTPEVTC | 25 |
| | VVVDVSHEDPEVKFNWYVDGVEVHNAKTKPREEQYNSTYRVVSVL | |
| | TVLHQDWLNGKEYKCKVSNKALPAPIEKTISKAKGQPREPQVYTL | |
| | PPSRDELTKNQVSLTCLVKGFYPSDIAVEWESNGQPENNYKTTPP | |
| | VLDSDGSFFLYSKLTVDKSRWQQGNVFSCSVMHEALHNHYTQKSL | |
| | SLSPGK | |
| hIgG1 | EPKSCDKTHTCPPCPAPPVAGPSVFLFPPKPKDTLMISRTPEVTC | 26 |
| PVA/P329A Fc | VVVDVSHEDPEVKFNWYVDGVEVHNAKTKPREEQYNSTYRVVSVL | |
| | TVLHQDWLNGKEYKCKVSNKALAAPIEKTISKAKGQPREPQVYTL | |
| | PPSRDELTKNQVSLTCLVKGFYPSDIAVEWESNGQPENNYKTTPP | |
| | VLDSDGSFFLYSKLTVDKSRWQQGNVFSCSVMHEALHNHYTQKSL | |
| | SLSPGK | |
| hIgG1 LALAPG | EPKSCDKTHTCPPCPAPEAAGGPSVFLFPPKPKDTLMISRTPEVT | 27 |
| Fc | CVVVDVSHEDPEVKFNWYVDGVEVHNAKTKPREEQYNSTYRVVSV | |
| | LTVLHQDWLNGKEYKCKVSNKALGAPIEKTISKAKGQPREPQVYT | |
| | LPPSRDELTKNQVSLTCLVKGFYPSDIAVEWESNGQPENNYKTTP | |
| | PVLDSDGSFFLYSKLTVDKSRWQQGNVFSCSVMHEALHNHYTQKS | |
| | LSLSPGK | |
| hIgG1 PVA star | EPKSCDKTHTCPPCPAPPVAGPSVFLFPPKPKDTLMISRTPEVTC | 28 |
| | VVVDVSHEDPEVKFNWYVDGVEVHNAKTKPREEQYNSTYRVVSVL | |
| | TVLHQDWLNGKEYKCKVSNKALPAPIEKTISKAKGQPREPQVYTL | |
| | PPSRDELTKNQVSLTCLVKGFYPSDIAVEWESNGQPENNYKTTPP | |
| | VLDSDGSFFLYSKLTVDKSRWQQGNVFSCSVMHEALHNRFTQKSL | |
| | SLSPGK | |
| hIgG1s | DKKVEPKSCDKTHTCPPCPAPPVAGPSVFLFPPKPKDTLMISRTP | 29 |
| | EVTCVVVDVSQEDPEVQFNWYVDGVEVHNAKTKPREEQFNSTYRV | |
| | VSVLTVLHQDWLNGKEYKCKVSNKGLPSSIEKTISKAKGQPREPQ | |
| | VYTLPPSRDELTKNQVSLTCLVKGFYPSDIAVEWESNGQPENNYK | |
| | TTPPVLDSDGSFFLYSKLTVDKSRWQQGNVFSCSVMHEALHNHY | |
| | TQKSLSLSPGK | |
| hIgG1 N180G, | EPKSCDKTHTCPPCPAPELLGGPSVFLFPPKPKDTLMISRTPEVT | 30 |
| also referred to | CVVVDVSHEDPEVKFNWYVDGVEVHNAKTKPREEQYGSTYRVVSV | |
| as N297G | LTVLHQDWLNGKEYKCKVSNKALPAPIEKTISKAKGQPREPQVYT | |
| | LPPSRDELTKNQVSLTCLVKGFYPSDIAVEWESNGQPENNYKTTP | |
| | PVLDSDGSFFLYSKLTVDKSRWQQGNVFSCSVMHEALHNHYTQKS | |
| | LSLSPGK | |
| hIgG2 variant | ERKCCVECPPCPAPPVAGPSVFLFPPKPKDTLMISRTPEVTCVVV | 31 |
| | DVSHEDPEVQFNWYVDGVEVHNAKTKPREEQFNSTFRVVSVLTVV | |
| | HQDWLNGKEYKCKVSNKGLPAPIEKTISKTKGQPREPQVYTLPPS | |
| | REEMTKNQVSLTCLVKGFYPSDIAVEWESNGQPENNYKTTPPMLD | |
| | SDGSFFLYSKLTVDKSRWQQGNVFSCSVMHEALHNHYTQKSLSLS | |
| | PGK | |
| hIgG4 S108P | ESKYGPPCPPCPAPEFLGGPSVFLFPPKPKDTLMISRTPEVTCVV | 32 |
| | VDVSQEDPEVQFNWYVDGVEVHNAKTKPREEQFNSTYRVVSVLTV | |
| | LHQDWLNGKEYKCKVSNKGLPSSIEKTISKAKGQPREPQVYTLPP | |
| | SQEEMTKNQVSLTCLVKGFYPSDIAVEWESNGQPENNYKTTPPVL | |
| | DSDGSFFLYSRLTVDKSRWQEGNVFSCSVMHEALHNHYTQKSLSL | |
| | SLGK | |
| IgG1-T- | DKTHTCPPCPAPELLGGPSVFLFPPKPKDTLMISRTPEVTCVVVD | 5 |
| Hex(WT) | VSHEDPEVKFNWYVDGVEVHNAKTKPREEQYNSTYRVVSVLTVLH | |
| | QDWLNGKEYKCKVSNKALPAPIEKTISKAKGQPREPQVYTLPPSR | |
| | DELTKNQVSLTCLVKGFYPSDIAVEWESNGQPENNYKTTPPVLDS | |
| | DGSFFLYSKLTVDKSRWQQGNVFSCSVMHEALHNHYTQKSLDKST | |
| | GKPTLYNVSLVMSDTAGTCY | |
| IgG1-T- | EPKSCDKTHTCPPCPAPELLGGPSVFLFPPKPKDTLMISRTPEVT | 6 |
| Hex(Mut) | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSRDELTKNQVSLTCLVKGFYPSDIAVEWESNGQPENNYKTTP | |
| | PVLDSDGSFFLYSKLTVDKSRWQQGNVFSCSVMHEALHNHYTQKS | |
| | LDKSTGKPTLYNVSLVMSDTAGTCY | |
| IgG1-T- | DKTHTCPPCPAPELLGGPSVFLFPPKPKDTLMISRTPEVTCVVVD | 51 |
| Hex(Mut-V2) | VSHEDPEVKFNWYVDGVEVHNAKTKPREEQYNSTYRVVSVLTVLH | |
| | QDWLNGKEYKCKVSNKALPAPIEKTISKAKGQPREPQVYTLPPSR | |
| | DELTKNQVSLTCLVKGFYPSDIAVEWESNGQPENNYKTTPPVLDS | |
| | DGSFFLYSKLTVDKSRWQQGNVFSCSVMHEALHNHYTQKSLDKST | |
| | GKPTLYNVSLVMSDTAGTCY | |
| IgG1-T- | DKTHTCPPCPAPELLGGPSVFLFPPKPKDTLMISRTPEVTCVVVD | 7 |
| Hex(Mut1) | VSHEDPEVKFNWYVDGVEVHNAKTKPREEQYNSTYRVVSVLTVCH | |
| | QDWLNGKEYKCKVSNKALPAPIEKTISKAKGQPREPQVYTLPPSR | |
| | EEMTKNQVSLTCLVKGFYPSDIAVEWESNGQPENNYKTTPPVLDS | |
| | DGSFFLYSKLTVDKSRWQQGNVFSCSVMHEALHNHYTQKSLDKST | |
| | GKPTLYNVSLIMSDTGGTCY | |
| IgG1-T- | DKTHTCPPCPAPELLGGPSVFLFPPKPKDTLMISRTPEVTCVVVD | 33 |
| Hex(Mut1-V2) | VSHEDPEVKFNWYVDGVEVHNAKTKPREEQYNSTYRVVSVLTVCH | |
| | QDWLNGKEYKCKVSNKALPAPIEKTISKAKGQPREPQVYTLPPSR | |
| | EEMTKNQVSLTCLVKGFYPSDIAVEWESNGQPENNYKTTPPVLDS | |
| | DGSFFLYSKLTVDKSRWQQGNVFSCSVMHEALHNHYTQKSLDKST | |
| | GKPTLYNVSLVMSDTAGTCY | |
| IgG1-T- | EPKSCDKTHTCPPCPAPELLGGPSVFLFPPKPKDTLMISRTPEVT | 52 |
| Hex(Mut1-V3) | CVVVDVSHEDPEVKFNWYVDGVEVHNAKTKPREEQYNSTYRVVSV | |
| | LTVCHQDWLNGKEYKCKVSNKALPAPIEKTISKAKGQPREPQVYT | |
| | LPPSREEMTKNQVSLTCLVKGFYPSDIAVEWESNGQPENNYKTTP | |
| | PVLDSDGSFFLYSKLTVDKSRWQQGNVFSCSVMHEALHNHYTQKS | |
| | LDKSTGKPTLYNVSLIMSDTGGTCY | |
| IgG1-T- | EPKSCDKTHTCPPCPAPELLGGPSVFLFPPKPKDTLMISRTPEVT | 53 |
| Hex(Mut1-V4) | CVVVDVSHEDPEVKFNWYVDGVEVHNAKTKPREEQYNSTYRVVSV | |
| | LTVCHQDWLNGKEYKCKVSNKALPAPIEKTISKAKGQPREPQVYT | |
| | LPPSREEMTKNQVSLTCLVKGFYPSDIAVEWESNGQPENNYKTTP | |
| | PVLDSDGSFFLYSKLTVDKSRWQQGNVFSCSVMHEALHNHYTQKS | |
| | LDKSTGKPTLYNVSLVMSDTAGTCY | |
| IgG1-T- | DKTHTCPPCPAPELLGGPSVFLFPPKPKDTLMCSRTPEVTCVVVD | 8 |
| Hex(Mut2) | VSHEAPEVKFNWYVDGVEVHNAKTKPREEQYNSTYRVVSVLTVLH | |
| | QDWLNGKEYKCKVSNKALPAGIEKTISKAKGQPREPQVYTLPPSR | |
| | DELTKNQVSLTCLVKGFYPSDIAVEWESNGQPENNYKTTPPVLDS | |
| | DGSFFLYSKLTVDKSRWQQGNVFSCSVMHEALHNHYTQKSLSLSP | |
| | GKPTLYNVSLVMSDTAGTCY | |
| IgG1-T- | EPKSCDKTHTCPPCPAPELLGGPSVFLFPPKPKDTLMCSRTPEVT | 54 |
| Hex(Mut2-V2) | CVVVDVSHEAPEVKFNWYVDGVEVHNAKTKPREEQYNSTYRVVSV | |
| | LTVLHQDWLNGKEYKCKVSNKALPAGIEKTISKAKGQPREPQVYT | |
| | LPPSRDELTKNQVSLTCLVKGFYPSDIAVEWESNGQPENNYKTTP | |
| | PVLDSDGSFFLYSKLTVDKSRWQQGNVFSCSVMHEALHNHYTQKS | |
| | LSLSPGKPTLYNVSLVMSDTAGTCY | |
| IgG1-T-Mut1- | DKTHTCPPCPAPEAAGGPSVFLFPPKPKDTLMISRTPEVTCVVVD | 9 |
| V2 LALAPG | VSHEDPEVKFNWYVDGVEVHNAKTKPREEQYNSTYRVVSVLTVCH | |
| | QDWLNGKEYKCKVSNKALGAPIEKTISKAKGQPREPQVYTLPPSR | |
| | EEMTKNQVSLTCLVKGFYPSDIAVEWESNGQPENNYKTTPPVLDS | |
| | DGSFFLYSKLTVDKSRWQQGNVFSCSVMHEALHNHYTQKSLDKST | |
| | GKPTLYNVSLVMSDTAGTCY | |
| IgG1-T-Mut1- | EPKSCDKTHTCPPCPAPEAAGGPSVFLFPPKPKDTLMISRTPEVT | 55 |
| V2 LALAPG | CVVVDVSHEDPEVKFNWYVDGVEVHNAKTKPREEQYNSTYRVVSV | |
| (V2) | LTVCHQDWLNGKEYKCKVSNKALGAPIEKTISKAKGQPREPQVYT | |
| | LPPSREEMTKNQVSLTCLVKGFYPSDIAVEWESNGQPENNYKTTP | |
| | PVLDSDGSFFLYSKLTVDKSRWQQGNVFSCSVMHEALHNHYTQKS | |
| | LDKSTGKPTLYNVSLVMSDTAGTCY | |
| IgG1-T-Mut1 | DKTHTCPPCPAPPVAGPSVFLFPPKPKDTLMISRTPEVTCVVVDV | 10 |
| PVA/P329A | SHEDPEVKFNWYVDGVEVHNAKTKPREEQYNSTYRVVSVLTVCHQ | |
| | DWLNGKEYKCKVSNKALAAPIEKTISKAKGQPREPQVYTLPPSRE | |
| | EMTKNQVSLTCLVKGFYPSDIAVEWESNGQPENNYKTTPPVLDSD | |
| | GSFFLYSKLTVDKSRWQQGNVFSCSVMHEALHNHYTQKSLDKSTG | |
| | KPTLYNVSLVMSDTAGTCY | |
| IgG1-T-Mut1 | EPKSCDKTHTCPPCPAPPVAGPSVFLFPPKPKDTLMISRTPEVTC | 56 |
| PVA/P329A | VVVDVSHEDPEVKFNWYVDGVEVHNAKTKPREEQYNSTYRVVSVL | |
| (V2) | TVCHQDWLNGKEYKCKVSNKALAAPIEKTISKAKGQPREPQVYTL | |
| | PPSREEMTKNQVSLTCLVKGFYPSDIAVEWESNGQPENNYKTTPP | |
| | VLDSDGSFFLYSKLTVDKSRWQQGNVFSCSVMHEALHNHYTQKSL | |
| | DKSTGKPTLYNVSLVMSDTAGTCY | |
| IgG1-T-Mut2 | DKTHTCPPCPAPEAAGGPSVFLFPPKPKDTLMCSRTPEVTCVVVD | 11 |
| LALAPG | VSHEAPEVKFNWYVDGVEVHNAKTKPREEQYNSTYRVVSVLTVLH | |
| | QDWLNGKEYKCKVSNKALGAGIEKTISKAKGQPREPQVYTLPPSR | |
| | DELTKNQVSLTCLVKGFYPSDIAVEWESNGQPENNYKTTPPVLDS | |
| | DGSFFLYSKLTVDKSRWQQGNVFSCSVMHEALHNHYTQKSLSLSP | |
| | GKPTLYNVSLVMSDTAGTCY | |
| IgG1-T-Mut2 | EPKSCDKTHTCPPCPAPEAAGGPSVFLFPPKPKDTLMCSRTPEVT | 57 |
| LALAPG (V2) | CVVVDVSHEAPEVKFNWYVDGVEVHNAKTKPREEQYNSTYRVVSV | |
| | LTVLHQDWLNGKEYKCKVSNKALGAGIEKTISKAKGQPREPQVYT | |
| | LPPSRDELTKNQVSLTCLVKGFYPSDIAVEWESNGQPENNYKTTP | |
| | PVLDSDGSFFLYSKLTVDKSRWQQGNVFSCSVMHEALHNHYTQKS | |
| | LSLSPGKPTLYNVSLVMSDTAGTCY | |
| IgG4/IgG1-T- | ESKYGPPCPPCPAPGGGGPSVFLFPPKPKDTLMISRTPEVTCVVV | 12 |
| Mut1 | DVSQEDPEVQFNWYVDGVEVHNAKTKPREEQFNSTYRVVSVLTVC | |
| | HQDWLNGKEYKCKVSNKGLPSSIEKTISKAKGQPREPQVYTLPPS | |
| | REEMTKNQVSLTCLVKGFYPSDIAVEWESNGQPENNYKTTPPVLD | |
| | SDGSFFLYSKLTVDKSRWQQGNVFSCSVMHEALHNHYTQKSLDKS | |
| | TGKPTLYNVSLVMSDTAGTCY | |
| IgG4/IgG1-T- | PPCPPCPAPGGGGPSVFLFPPKPKDTLMISRTPEVTCVVVDVSQE | 58 |
| Mut1 (V2) | DPEVQFNWYVDGVEVHNAKTKPREEQFNSTYRVVSVLTVCHQDWL | |
| | NGKEYKCKVSNKGLPSSIEKTISKAKGQPREPQVYTLPPSREEMT | |
| | KNQVSLTCLVKGFYPSDIAVEWESNGQPENNYKTTPPVLDSDGSF | |
| | FLYSKLTVDKSRWQQGNVFSCSVMHEALHNHYTQKSLDKSTGKPT | |
| | LYNVSLVMSDTAGTCY | |

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:1, exclusive or inclusive of C-terminal substitutions described in Section 5.4.1.5. In cases where an Fc domain comprises at least 90% sequence identity and less than 100% sequence identity to SEQ ID NO:1 (e.g., between 90% and 99% sequence identity to SEQ ID NO:1), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:5, exclusive or inclusive of C-terminal substitutions described in Section 5.4.1.5. In cases where an Fc domain comprises at least 90% sequence identity and less than 100% sequence identity to SEQ ID NO:5 (e.g., between 90% and 99% sequence identity to SEQ ID NO:5), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:6, exclusive or inclusive of C-terminal substitutions described in Section 5.4.1.5. In cases where an Fc domain comprises at least 90% sequence identity and less than 100% sequence identity to SEQ ID NO:6 (e.g., between 90% and 99% sequence identity to SEQ ID NO:6), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:7, exclusive or inclusive of C-terminal substitutions described in Section 5.4.1.5. In cases where an Fc domain comprises at least 90% sequence identity and less than 100% sequence identity to SEQ ID NO:7 (e.g., between 90% and 99% sequence identity to SEQ ID NO:7), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:8, exclusive or inclusive of C-terminal substitutions described in Section 5.4.1.5. In cases where an Fc domain comprises at least 90% sequence identity and less than 100% sequence identity to SEQ ID NO:8 (e.g., between 90% and 99% sequence identity to SEQ ID NO:8), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:9, exclusive or inclusive of C-terminal substitutions described in Section 5.4.1.5. In cases where an Fc domain comprises at least 90% sequence identity and less than 100% sequence identity to SEQ ID NO:9 (e.g., between 90% and 99% sequence identity to SEQ ID NO:9), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO: 1, exclusive or inclusive of C-terminal substitutions described in Section 5.4.1.5. In cases where an Fc domain comprises at least 90% sequence identity and less than 100% sequence identity to SEQ ID NO: 1 (e.g., between 90% and 99% sequence identity to SEQ ID NO:1), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO: 10, exclusive or inclusive of C-terminal substitutions described in Section 5.4.1.5. In cases where an Fc domain comprises at least 90% sequence identity and less than 100% sequence identity to SEQ ID NO: 10 (e.g., between 90% and 99% sequence identity to SEQ ID NO: 10), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO: 11, exclusive or inclusive of C-terminal substitutions described in Section 5.4.1.5. In cases where an Fc domain comprises at least 90% sequence identity and less than 100% sequence identity to SEQ ID NO: 11 (e.g., between 90% and 99% sequence identity to SEQ ID NO: 11), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO: 12, exclusive or inclusive of C-terminal substitutions described in Section 5.4.1.5. In cases where an Fc domain comprises at least 90% sequence identity and less than 100% sequence identity to SEQ ID NO: 12 (e.g., between 90% and 99% sequence identity to SEQ ID NO: 12), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:21, exclusive or inclusive of C-terminal substitutions described in Section 5.4.1.5. In cases where an Fc domain comprises at least 90% sequence identity and less than 100% sequence identity to SEQ ID NO: 21 (e.g., between 90% and 99% sequence identity to SEQ ID NO:21), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:22, exclusive or inclusive of C-terminal substitutions described in Section 5.4.1.5. In cases where an Fc domain comprises at least 90% sequence identity and less than 100% sequence identity to SEQ ID NO: 22 (e.g., between 90% and 99% sequence identity to SEQ ID NO:22), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:23, exclusive or inclusive of C-terminal substitutions described in Section 5.4.1.5. In cases where an Fc domain comprises at least 90% sequence identity and less than 100% sequence identity to SEQ ID NO: 23 (e.g., between 90% and 99% sequence identity to SEQ ID NO:23), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:24, exclusive or inclusive of C-terminal substitutions described in Section 5.4.1.5. In cases where an Fc domain comprises at least 90% sequence identity and less than 100% sequence identity to SEQ ID NO: 24 (e.g., between 90% and 99% sequence identity to SEQ ID NO:24), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:25, exclusive or inclusive of C-terminal substitutions described in Section 5.4.1.5. In cases where an Fc domain comprises at least 90% sequence identity and less than 100% sequence identity to SEQ ID NO: 25 (e.g., between 90% and 99% sequence identity to SEQ ID NO:25), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:26, exclusive or inclusive of C-terminal substitutions described in Section 5.4.1.5. In cases where an Fc domain comprises at least 90% sequence identity and less than 100% sequence identity to SEQ ID NO: 26 (e.g., between 90% and 99% sequence identity to SEQ ID NO:26), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:27, exclusive or inclusive of C-terminal substitutions described in Section 5.4.1.5. In cases where an Fc domain comprises at least 90% sequence identity and less than 100% sequence identity to SEQ ID NO: 27 (e.g., between 90% and 99% sequence identity to SEQ ID NO:27), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:28, exclusive or inclusive of C-terminal substitutions described in Section 5.4.1.5. In cases where an Fc domain comprises at least 90% sequence identity and less than 100% sequence identity to SEQ ID NO: 28 (e.g., between 90% and 99% sequence identity to SEQ ID NO:28), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:29, exclusive or inclusive of C-terminal substitutions described in Section 5.4.1.5. In cases where an Fc domain comprises at least 90% sequence identity and less than 100% sequence identity to SEQ ID NO: 29 (e.g., between 90% and 99% sequence identity to SEQ ID NO:29), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:30, exclusive or inclusive of C-terminal substitutions described in Section 5.4.1.5. In cases where an Fc domain comprises at least 90% sequence identity and less than 100% sequence identity to SEQ ID NO: 30 (e.g., between 90% and 99% sequence identity to SEQ ID NO:30), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:31, exclusive or inclusive of C-terminal substitutions described in Section 5.4.1.5. In cases where an Fc domain comprises at least 90% sequence identity and less than 100% sequence identity to SEQ ID NO: 31 (e.g., between 90% and 99% sequence identity to SEQ ID NO:31), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:32, exclusive or inclusive of C-terminal substitutions described in Section 5.4.1.5. In cases where an Fc domain comprises at least 90% sequence identity and less than 100% sequence identity to SEQ ID NO: 32 (e.g., between 90% and 99% sequence identity to SEQ ID NO:32), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:51, exclusive or inclusive of C-terminal substitutions described in Section 5.4.1.5. In cases where an Fc domain comprises at least 90% sequence identity and less than 100% sequence identity to SEQ ID NO: 51 (e.g., between 90% and 99% sequence identity to SEQ ID NO:51), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:52, exclusive or inclusive of C-terminal substitutions described in Section 5.4.1.5. In cases where an Fc domain comprises at least 90% sequence identity and less than 100% sequence identity to SEQ ID NO: 52 (e.g., between 90% and 99% sequence identity to SEQ ID NO:52), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:53, exclusive or inclusive of C-terminal substitutions described in Section 5.4.1.5. In cases where an Fc domain comprises at least 90% sequence identity and less than 100% sequence identity to SEQ ID NO: 53 (e.g., between 90% and 99% sequence identity to SEQ ID NO:53), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:54, exclusive or inclusive of C-terminal substitutions described in Section 5.4.1.5. In cases where an Fc domain comprises at least 90% sequence identity and less than 100% sequence identity to SEQ ID NO: 54 (e.g., between 90% and 99% sequence identity to SEQ ID NO:54), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:55, exclusive or inclusive of C-terminal substitutions described in Section 5.4.1.5. In cases where an Fc domain comprises at least 90% sequence identity and less than 100% sequence identity to SEQ ID NO: 55 (e.g., between 90% and 99% sequence identity to SEQ ID NO:55), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:56, exclusive or inclusive of C-terminal substitutions described in Section 5.4.1.5. In cases where an Fc domain comprises at least 90% sequence identity and less than 100% sequence identity to SEQ ID NO: 56 (e.g., between 90% and 99% sequence identity to SEQ ID NO:56), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:57, exclusive or inclusive of C-terminal substitutions described in Section 5.4.1.5. In cases where an Fc domain comprises at least 90% sequence identity and less than 100% sequence identity to SEQ ID NO: 57 (e.g., between 90% and 99% sequence identity to SEQ ID NO:57), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

In some aspects, an Fc domain comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO:58, exclusive or inclusive of C-terminal substitutions described in Section 5.4.1.5. In cases where an Fc domain comprises at least 90% sequence identity and less than 100% sequence identity to SEQ ID NO: 58 (e.g., between 90% and 99% sequence identity to SEQ ID NO:58), an Fc domain may also comprise one or more amino acid substitutions described herein, for example one or more substitutions that alter effector function (e.g., as described in Section 5.4.1) and/or one or more substitutions that promote Fc heterodimerization (e.g., as described in Section 5.4.1.4).

The two Fc domains within a dimer of the VEGF antagonist core of the disclosure can be the same or different from one another. In a native antibody, the Fc domains are typically identical; however, in certain applications having different Fc domains might be advantageous, such as for the purpose of producing multispecific binding molecules and/or generating heterodimers with distinct effector functions, as described in Sections 5.4.1 and 5.4.1.4 below.

In one embodiment the Fc domain comprises CH2 and CH3 domains derived from IgG1.

In one embodiment the Fc domain comprises CH2 and CH3 domains derived from IgG2.

In one embodiment the Fc domain comprises CH2 and CH3 domains derived from IgG3.

In one embodiment the Fc domain comprises CH2 and CH3 domains derived from IgG4.

It will be appreciated that the Fc domains for use in producing the VEGF antagonists of the present disclosure may include variants of the naturally occurring Fc domains described above. Such variants may comprise one or more amino acid variations compared to wild type Fc domains. In one example, an Fc domain of the present disclosure varies in sequence from the wildtype Fc domain. It will be appreciated that the variant Fc domains may be longer or shorter than the wild-type Fc domain. Preferably the variant Fc domains are at least 60% identical or similar to a wild-type Fc domain. In another example the variant Fc domains are at least 70% identical or similar. In another example the variant Fc domains are at least 80% identical or similar. In another example the variant Fc domains are at least 90% identical or similar. In another example the variant Fc domains are at least 95% identical or similar.

In some embodiments, the C-terminal amino acids of the IgG Fc domains include one or more mutations (e.g., amino acid substitutions, deletions and/or inserts) to effectively replace the C-terminal amino acids of the IgG Fc domains with those of an IgM CH4 (Cμ4) domain. In various embodiments, (a) 5, 6, 7, 8, 9 or all 10 of the 10 C-terminal amino acids of the IgG Fc correspond to those of an IgM CH4 (Cμ4) domain and/or (b) all 4, 5, or 6 C-terminal amino acids correspond to those of an IgM CH4 (Cμ4) domain. An exemplary amino acid sequence at the C-terminus of an IgG Fc domain preceding the tailpiece is DKSTGK (SEQ ID NO: 72), which in some embodiments directly precedes the tailpiece without an intervening linker sequence.

The Fc domains that are incorporated into the VEGF antagonists of the present disclosure can comprise one or more modifications that alter the functional properties of the proteins, for example, binding to Fc-receptors such as FcRn or leukocyte receptors, binding to complement, modified disulfide bond architecture, or altered glycosylation patterns. Exemplary Fc modifications that alter effector function are described in Section 5.4.1.3. Exemplary modifications to disulfide bond architecture are disclosed in Section 5.4.1.1.

The Fc domains can also be altered to include modifications that improve manufacturability of heterodimers to be included in VEGF antagonists. Heterodimerization is the preferential pairing of non-identical Fc domains over identical Fc domains and permits the production of VEGF antagonists in which individual dimers comprise polypeptide components that differ in sequence. Examples of heterodimerization strategies are exemplified in Section 5.4.1.4.

It will be appreciated that any of the modifications mentioned above can be combined in any suitable manner to achieve the desired functional properties and/or combined with other modifications to alter the properties of the VEGF antagonists.

5.4.1.1. Fc Domains with Engineered Disulfides

In some embodiments, the Fc domain comprises one or more amino acid substitutions that facilitate formation of an engineered disulfide bond between two Fc domains of an Fc region. An “engineered disulfide bond” describes a disulfide bond between two Fc domains (e.g., between two CH2 domains or between two CH3 domains) which is not present in a natural immunoglobulin of the same type (e.g., IgG1, IgG2, IgG3, IgG4). In general, such a substitution comprises replacement of a non-cysteine residue with a cysteine residue on one or both Fc domains of an Fc region, facilitating formation of a disulfide bond between two cysteine residues.

In some embodiments, the Fc domain comprises a cysteine residue at position 309, EU numbering (L309C). In some embodiments, the Fc domain comprises a cysteine residue at position 253, EU numbering (I253C). In some embodiments, the Fc domain comprises a cysteine residue at position 253 (I253C) and a cysteine residue at position 309 (L309C), EU numbering. Such substitutions can be combined with one or more additional Fc domain substitutions known in the art or described herein, for example they can be used in chimeric Fc domains as described in Section 5.4.1.2, together with substitutions conferring altered effector function as described in Section 5.4.1.3, and/or together with C-terminal substitutions as described in Section 5.4.1.5.

In some embodiments, the CH2 and/or CH3 domains in an Fc region of a VEGF antagonist of the disclosure comprise one or more engineered cysteines designed to form disulfide bonds, for example between corresponding I253C or L309C mutations within an Fc pair.

In other embodiments, the CH2 and CH3 domains in an Fc region of a VEGF antagonist of the disclosure comprises do not include engineered cysteines as compared to a wild-type Fc domain, e.g., wild-type IgG1, IgG2, IgG3, or IgG4, that result in the formation of additional disulfide bonds. Accordingly, in some embodiments, the CH2 and CH3 domains in an Fc region of a VEGF antagonist of the disclosure include only native cysteines (which can be all or a subset of the native cysteines) and/or native disulfide bonding (to the extent present) as compared to a wild-type Fc domain. Exemplary wild-type IgG1, IgG2, IgG3, and IgG4 domains are presented in SEQ ID NO:1, SEQ ID NO:21, SEQ ID NO:22, and SEQ ID NO:23, respectively.

### 5.4.1.2. Chimeric Fc Domains

In some embodiments, an Fc domain disclosed herein is a chimeric Fc domain. Chimeric Fc domains of the disclosure comprise an IgG1 upper hinge domain, an IgG1 lower hinge domain having the substitution/deletion mutation ELLG→PVA-(“ELLG” disclosed as SEQ ID NO: 73) (where “-” represents an unoccupied position) at amino acid positions 233-236 (EU numbering), an IgG1 CH2 domain, and an IgG1 CH3 domain. In some embodiments, a chimeric Fc domain also comprises an IgG1 CH1 domain or a fragment thereof.

IgG1 Fc domains with a hinge region modified to reduce Fc receptor and/or effector function are provided. The modification occurs at amino acid positions 233-236 (EU numbering) by substitution/deletion of ELLG (SEQ ID NO: 73) with PVA-, where amino acid 236 is deleted. In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

Excluding the ELLG→PVA-substitution/deletion at amino acid positions 233-236 (“ELLG” disclosed as SEQ ID NO: 73) (EU numbering), a chimeric Fc domain is considered to be of an IgG1 isotype if it differs from IgG1 by no more than 1, 2, 3, 4, 5, 6, 7, 8, 9, 10, 11, 12, 13, 14, 15, 16, 17, 18, 19, or 20 substitutions, deletions or insertions, except however, that the CH1 domain can optionally be omitted entirely, as can the upper hinge region. CH1, CH2 and CH3 domains are each considered to be of IgG1 isotype if differing from the CH1, CH2 and CH3 region of the IgG1 wild type sequence by no more than 1, 2, 3, 4, 5, 6, 7, 8, 9, or 10 substitutions, deletions, or insertions. Substitutions, deletion, and/or insertions (excluding the ELLG→PVA-substitution/deletion at amino acid positions 233-236 (“ELLG” disclosed as SEQ ID NO: 73) (EU numbering)) can be any substitutions, deletions, and/or insertions. In some embodiments, the substitutions, deletion, and/or insertions do not result in a sequence identical to, e.g., CH1, CH2, or CH3 of another IgG isotype (e.g., IgG2, IgG3, or IgG4). For example, in some embodiments, if a chimeric Fc domain of the disclosure comprises one or more of H268Q, K274Q, Y296F, A327G, A330S, and P331S (EU numbering), they do not all occur simultaneously.

The sequence of wild type IgG1 Fc domain (amino acids 216-447; EU numbering)) comprises:

(SEQ ID NO: 1)
EPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVL
HQDWLNGKEYKCKVSNKALPAPIEKTISKAKGQPREPQVYTLPPSRD
ELTKNQVSLTCLVKGFYPSDIAVEWESNGQPENNYKTTPPVLDSDGS
FFLYSKLTVDKSRWQQGNVFSCSVMHEALHNHYTQKSLSLSPGK,

where the upper hinge region comprises EPKSCDKTHT (SEQ ID NO: 74) (amino acids 216-225 (EU numbering); amino acids 1-10 of the wild type IgG1 Fc domain sequence); the core hinge comprises CPPC (SEQ ID NO: 75) (amino acids 226-229 (EU numbering); amino acids 11-14 of the wild type IgG1 Fc domain sequence); the lower hinge region comprises PAPELLG (SEQ ID NO: 76) (amino acids 230-236 (EU numbering); amino acids 15-21 of the wild type IgG1 Fc domain sequence), CH2 comprises GPSVFLFPPKPKDTLMISRTPEVTCVVVDVSHEDPEVKFNWYVDGVEVHNAKTKPREEQYNST YRVVSVLTVLHQDWLNGKEYKCKVSNKALPAPIEKTISKAK (SEQ ID NO: 77) (amino acids 237-340 (EU numbering); amino acids 22-125 of the wild type IgG1 Fc domain sequence), and CH3 comprises GQPREPQVYTLPPSRDELTKNQVSLTCLVKGFYPSDIAVEWESNGQPENNYKTTPPVLDSDGS FFLYSKLTVDKSRWQQGNVFSCSVMHEALHNHYTQKSLSLSPGK (SEQ ID NO: 78) (amino acids 341-447 (EU numbering); amino acids 126-232 of the wild type IgG1 Fc sequence). It will be recognized by those skilled in the art that amino acids 237-238 (EU numbering) represents both the C-terminus of the lower hinge and the N-terminus of the CH2 region. For the purposes of the instant disclosure, however, amino acids 237-238 (EU numbering) are shown as part of the lower hinge.

In some embodiments, the sequence of the chimeric Fc region of the disclosure, also termed IgG1 PVA herein (amino acids 216-447; EU numbering)) comprises:

(SEQ ID NO: 25)
EPKSCDKTHTCPPCPAPPVAGPSVFLFPPKPKDTLMISRTPEVTCVV
VDVSHEDPEVKFNWYVDGVEVHNAKTKPREEQYNSTYRVVSVLTVLH
QDWLNGKEYKCKVSNKALPAPIEKTISKAKGQPREPQVYTLPPSRDE
LTKNQVSLTCLVKGFYPSDIAVEWESNGQPENNYKTTPPVLDSDGSF
FLYSKLTVDKSRWQQGNVFSCSVMHEALHNHYTQKSLSLSPGK,

where the upper hinge region comprises EPKSCDKTHT (SEQ ID NO: 74) (amino acids 216-225 (EU numbering); amino acids 1-10 of the IgG1 PVA Fc domain sequence); the core hinge comprises CPPC (SEQ ID NO: 75) (amino acids 226-229 (EU numbering); amino acids 11-14 of the IgG1 PVA Fc domain sequence); the lower hinge region comprises PAPPVA (SEQ ID NO: 79) (amino acids 230-236 (EU numbering); amino acids 15-20 of the IgG1 PVA Fc domain sequence), CH2 comprises GPSVFLFPPKPKDTLMISRTPEVTCVVVDVSHEDPEVKFNWYVDGVEVHNAKTKPREEQYNST YRVVSVLTVLHQDWLNGKEYKCKVSNKALPAPIEKTISKAK (SEQ ID NO: 77) (amino acids 237-340 (EU numbering); amino acids 21-124 of the IgG1 PVA sequence), and CH3 comprises GQPREPQVYTLPPSRDELTKNQVSLTCLVKGFYPSDIAVEWESNGQPENNYKTTPPVLDSDGS FFLYSKLTVDKSRWQQGNVFSCSVMHEALHNHYTQKSLSLSPGK (SEQ ID NO: 78) (amino acids 341-447 (EU numbering); amino acids 125-231 of the IgG1 PVA sequence).

In some aspects, the chimeric Fc domain comprises an amino acid sequence having at least 90%, at least 91%, at least 92%, at least 93%, at least 94%, at least 95%, at least 96%, at least 97%, at least 98%, at least 98%, or 100% sequence identity with:

(SEQ ID NO: 25)
EPKSCDKTHTCPPCPAPPVAGPSVFLFPPKPKDTLMISRTPEVTCVV
VDVSHEDPEVKFNWYVDGVEVHNAKTKPREEQYNSTYRVVSVLTVLH
QDWLNGKEYKCKVSNKALPAPIEKTISKAKGQPREPQVYTLPPSRDE
LTKNQVSLTCLVKGFYPSDIAVEWESNGQPENNYKTTPPVLDSDGSF
FLYSKLTVDKSRWQQGNVFSCSVMHEALHNHYTQKSLSLSPGK.

In some embodiments, the chimeric Fc domain comprises one or more amino acid substitutions in addition to the ELLG→PVA-substitution/deletion at amino acid positions 233-236 (“ELLG” disclosed as SEQ ID NO: 73) (EU numbering), with the additional amino acid substitutions further reducing binding to an Fc receptor and/or effector function.

In some embodiments, the chimeric Fc domain lacks up to five amino acids from the N-terminus of the amino acid sequence relative to the sequence of SEQ ID NO:25. For example, in some embodiments, the chimeric Fc domain comprises an amino acid sequence having at least 90%, at least 91%, at least 92%, at least 93%, at least 94%, at least 95%, at least 96%, at least 97%, at least 98%, at least 98%, or 100% sequence identity with the amino acid sequence of SEQ ID NO:25 minus the first five amino acids (EPKSC (SEQ ID NO: 80)).

In one embodiment, the chimeric Fc domain comprises one or more amino acid substitution at one or more of positions L234, L235, G237, D265, N297, P329, A330, P331, and P329 (EU numbering).

In some embodiments, the chimeric Fc domain comprises an amino acid substitution at position P329 (EU numbering). In a more specific embodiment, the amino acid substitution is P329A or P329G (EU numbering). In a particular embodiment, the chimeric Fc domain comprises, in addition to the ELLG→PVA-substitution/deletion at amino acid positions 233-236 (“ELLG” disclosed as SEQ ID NO: 73), the amino acid substitution P329A (EU numbering). Such a chimeric Fc domain is sometimes referred to herein as “IgG1 PVA/P329A,” an exemplary sequence of which is provided below:

(SEQ ID NO: 26)
EPKSCDKTHTCPPCPAPPVAGPSVFLFPPKPKDTLMISRTPEVTCVV
VDVSHEDPEVKFNWYVDGVEVHNAKTKPREEQYNSTYRVVSVLTVLH
QDWLNGKEYKCKVSNKALAAPIEKTISKAKGQPREPQVYTLPPSRDE
LTKNQVSLTCLVKGFYPSDIAVEWESNGQPENNYKTTPPVLDSDGSF
FLYSKLTVDKSRWQQGNVFSCSVMHEALHNHYTQKSLSLSPGK

Accordingly, in some embodiments, the chimeric Fc domain comprises an amino acid sequence having at least 90%, at least 91%, at least 92%, at least 93%, at least 94%, at least 95%, at least 96%, at least 97%, at least 98%, at least 98%, or 100% sequence identity with SEQ ID NO:26.

In one embodiment, the chimeric Fc domain comprises an amino acid substitution at position P329 and a further amino acid substitution at position N297 and/or P331 (EU numbering). In a more specific embodiment, the further amino acid substitution is N297A or N297D and/or P331S.

In some embodiments, the chimeric Fc domain comprises amino acid substitutions at positions G237, A330, and P331 (EU numbering). In a more specific embodiment, the amino acid substitutions are G237A, A330S, and P331S (EU numbering).

In some embodiments, the chimeric Fc domain comprises D265A and N297A mutations (EU numbering) to reduce effector function.

5.4.1.3. Fc Domains with Altered Effector Function

In some embodiments, the Fc domain comprises one or more amino acid substitutions that reduces binding to an Fc receptor and/or reduces effector function. In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

In one embodiment, the Fc domain comprises an amino acid substitution at a position selected from the group of E233, L234, L235, N297, P331 and P329 (EU numbering). In a more specific embodiment, the Fc domain comprises an amino acid substitution at a position selected from the group of L234, L235 and P329 (EU numbering index). In some embodiments, one or both Fc domains comprises the amino acid substitutions L234A and L235A (EU numbering). In one embodiment, the Fc domain comprises an amino acid substitution at position P329. In a more specific embodiment, the amino acid substitution is P329A or P329G (EU numbering). In one embodiment, the Fc domain comprises an amino acid substitution at position P329 and a further amino acid substitution at a position selected from E233, L234, L235, N297 and P331 (numberings according to Kabat EU index). In a more specific embodiment, the further amino acid substitution is E233P, L234A, L235A, L235E, N297A, N297D or P331S. In particular embodiments, the Fc domain or the Fc region comprises amino acid substitutions at positions P329, L234 and L235 (numberings according to Kabat EU index). In more particular embodiments, the Fc domain comprises the amino acid mutations L234A, L235A and P329G (“P329G LALA”, “PGLALA” or “LALAPG”).

In one embodiment, the Fc domain is an IgG1 Fc domain, particularly a human IgG1 Fc domain. In some embodiments, the IgG1 Fc domain is a variant IgG1 comprising D265A, N297A mutations (EU numbering) to reduce effector function.

In one embodiment, the Fc domain comprises one or more amino acid substitutions that decrease FcRn valency as compared to a wild-type IgG1 Fc domain. Exemplary amino acid residues that can be substituted to decrease FcRn valency include but are not limited to the amino acid isoleucine at position 253, histidine at position 310, and histidine at position 435 (EU numbering). Substitutions at all three positions are sometimes referred to as “IHH” substitutions.

In one embodiment, isoleucine at position 253 in an Fc domain is substituted with alanine.

In one embodiment, histidine at position 310 in an Fc domain is substituted with alanine.

In one embodiment, histidine at position 435 in an Fc domain is substituted with alanine, lysine or arginine. Substituting histidine 435 with arginine facilitates purification as described in Section 5.4.1.4.

Accordingly, in various embodiments, an Fc domain comprises one, two, or all three of the amino acid substitutions I253A, H310A, and H435A/K/R.

In another embodiment, the Fc domain is an IgG4 Fc domain (e.g., as set forth in SEQ ID NO: 23) or a variant thereof with reduced binding to Fc receptors. Exemplary IgG4 Fc domains with reduced binding to Fc receptors are exemplified by SEQ ID NO:24, SEQ ID NO: 32, and variants thereof as described herein.

### 5.4.1.4. Fc Heterodimerization Variants

Certain VEGF antagonists of the disclosure comprise, and entail heterodimerization between, two non-identical Fc domains. Inadequate heterodimerization of two Fc domains in a mixed Fc population to form an Fc region has can be an obstacle for increasing the yield of desired heterodimeric molecules and represents challenges for purification. A variety of approaches available in the art can be used in for enhancing dimerization of Fc domains in the VEGF antagonists of the disclosure, for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO 2009/089004A1.

Typically, each Fc domain in the Fc heterodimer comprises a CH3 domain of an antibody. The CH3 domains are derived from the Fc region of an antibody of any isotype, class or subclass, and preferably of IgG (IgG1, IgG2, IgG3 and IgG4) class, as described in the preceding section.

In embodiments involving VEGF antagonists with different Fc domains, heterodimerization of the two different Fc domains at CH3 domains gives rise to the desired VEGF antagonist, while homodimerization of identical Fc domains will reduce yield of the desired VEGF antagonist. Thus, in certain embodiments, the polypeptides that associate to form a heterodimeric Fc unit will contain CH3 domains with modifications that favor heterodimeric association relative to unmodified Fc domains.

In a specific embodiment said modification promoting the formation of Fc heterodimers is a so-called “knob-into-hole” or “knob-in-hole” modification, comprising a “knob” modification in one of the Fc domains and a “hole” modification in the other Fc domain. The knob-into-hole technology is described e.g., in U.S. Pat. Nos. 5,731,168; 7,695,936; Ridgway et al., 1996, Prot Eng 9:617-621, and Carter, 2001, Immunol Meth 248:7-15. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in some embodiments, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine(S), threonine (T), and valine (V). The protuberance and cavity can be made by altering the nucleic acid encoding the polypeptides, e.g., by site-specific mutagenesis, or by peptide synthesis. An exemplary substitution is Y470T.

In a specific such embodiment, in the first Fc domain the threonine residue at position 366 is replaced with a tryptophan residue (T366W), and in the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further embodiment, in the first Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second Fc domain additionally the tyrosine residue at position 349 is replaced by a cysteine residue (Y349C) (numbering according to Kabat EU index). In a particular embodiment, the first Fc domain comprises the amino acid substitutions S354C and T366W, and the second Fc domain comprises the amino acid substitutions Y349C, T366S, L368A and Y407V (numbering according to Kabat EU index).

In some embodiments, electrostatic steering (e.g., as described in Gunasekaran et al., 2010, J Biol Chem 285(25): 19637-46) can be used to promote the association of the first and the second Fc domains of the Fc region.

As an alternative, or in addition, to the use of Fc domains that are modified to promote heterodimerization, an Fc domain can be modified to allow a purification strategy that enables selections of Fc heterodimers. In one such embodiment, one polypeptide comprises a modified Fc domain that abrogates its binding to Protein A, thus enabling a purification method that yields a heterodimeric protein. See, for example, U.S. Pat. No. 8,586,713. As such, the VEGF antagonists comprise a first CH3 domain and a second Ig CH3 domain, wherein the first and second Ig CH3 domains differ from one another by at least one amino acid, and wherein at least one amino acid difference reduces binding of the VEGF antagonist to Protein A as compared to a corresponding VEGF antagonist lacking the amino acid difference. In one embodiment, the first CH3 domain binds Protein A and the second CH3 domain contains a mutation/modification that reduces or abolishes Protein A binding such as an H95R modification (by IMGT exon numbering; H435R by EU numbering). The second CH3 may further comprise a Y96F modification (by IMGT; Y436F by EU). This class of modifications is referred to herein as “star” mutations.

In some embodiments, the Fc domain can contain one or more mutations (e.g., knob and hole mutations) to facilitate heterodimerization as well as star mutations to facilitate purification.

### 5.4.1.5. C-Terminal Substitutions

The C-terminal amino acids of the Fc domains of the VEGF antagonists of the disclosure can include one or more mutations (e.g., amino acid substitutions, deletions and/or insertions) to effectively replace the C-terminal amino acids of the IgG Fc domains with those of an IgM CH4 (Cμ4) domain.

In various embodiments, (a) 5, 6, 7, 8, 9 or all 10 of the 10 C-terminal amino acids of the IgG Fc correspond to those of an IgM CH4 (Cμ4) domain and/or (b) all 4, 5, or 6 C-terminal amino acids correspond to those of an IgM CH4 (Cμ4) domain. Any substitutions in the last 10 amino acids of an Fc domain are referred to herein as “C-terminal substitutions” for convenience, notwithstanding that such substitutions are N-terminal to the tailpieces of the Fc domains of the disclosure.

An exemplary amino acid sequence at the C-terminus of an IgG Fc domain preceding the tailpiece is DKSTGK (SEQ ID NO: 72), which in some embodiments directly precedes the tailpiece without an intervening linker sequence.

### 5.4.2. Tailpiece (T)

In some embodiments, the multimerization domains of the disclosure comprise a tailpiece C-terminal to an Fc domain. The tailpiece in the multimerization domains of the disclosure can be derived from any suitable species. Antibody tailpieces are evolutionarily conserved and are found in most species, including primitive species such as teleosts. In one embodiment the tailpiece is derived from a human IgM antibody.

IgM occurs naturally in humans as a covalent multimer of the common H2L2 antibody unit. IgM occurs as a pentamer when it has incorporated a J-chain, or as a hexamer when it lacks a J-chain. The heavy chains of IgM possess an 18 amino acid extension to the C-terminal Fc domain, known as a tailpiece. This tailpiece includes a cysteine residue that forms a disulfide bond and is believed to be involved in polymerization of IgM Fc pairs. The tailpiece also contains a glycosylation site.

The tailpiece can be fused directly to the C-terminus of the Fc domain. Alternatively, it can be fused indirectly by means of an intervening amino acid sequence such as that of a short linker.

The tailpiece may include variants or fragments of a native IgM tailpiece, e.g., a variant having one or more mutations including but not limited to substitution, deletion or insertion mutations.

In some embodiments, the tailpiece comprises the amino acid sequence of any one of SEQ ID NOS: 2, 3 and 4, or an amino acid sequence having at least 85% or at least 90% sequence identity thereto. In some embodiments, a tailpiece that does not have 100% sequence identity to any one of SEQ ID NOS: 2, 3 and 4 retains the cysteine at position 17 of the 18-mer sequence.

In some embodiments, the tailpiece comprises the amino acid sequence of SEQ ID NO: 2, or an amino acid sequence having at least 85% or at least 90% sequence identity thereto. In some embodiments, the tailpiece comprises the amino acid sequence of SEQ ID NO: 3. In some embodiments, the tailpiece comprises the amino acid sequence of SEQ ID NO:4.

In some embodiments, the C-terminal amino acids of the IgG Fc domain include one or more mutations (e.g., amino acid substitutions, deletions and/or inserts) to effectively replace the C-terminal amino acids of the IgG Fc with those of an IgM CH4 (Cμ4) domain. In various embodiments, (a) 5, 6, 7, 8, 9 or all 10 of the 10 C-terminal amino acids of the IgG Fc correspond to those of an IgM CH4 (Cμ4) domain and/or (b) all 4, 5, or 6 C-terminal amino acids correspond to those of an IgM CH4 (Cμ4) domain. An exemplary amino acid sequence at the C-terminus of the IgG Fc domain preceding the tailpiece is DKSTGK (SEQ ID NO: 72), which in some embodiments directly precedes the tailpiece without an intervening linker sequence.

### 5.5. Linkers

In certain aspects, the present disclosure provides VEGF antagonists in which two or more components are connected to one another by a peptide linker.

By way of example and not limitation, linkers can be used to connect a VEGF binding domain to the hinge domain of an IgG Fc domain, an IgM tailpiece sequence to the C-terminus of an IgG Fc domain, and/or a VEGF target binding domain to a VEGF target binding domain.

A peptide linker can range from 2 amino acids to 60 or more amino acids, and in certain aspects a peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length.

In particular aspects, a peptide linker is at least 5 amino acids, at least 6 amino acids or at least 7 amino acids in length and optionally is up to 30 amino acids, up to 40 amino acids, up to 50 amino acids or up to 60 amino acids in length.

In some embodiments of the foregoing, the linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

In some embodiments, the linker is a GGGGS (G4S) linker (SEQ ID NO: 71). In some embodiments the linker comprises two consecutive G4S sequences (SEQ ID NO: 81), three consecutive G4S sequences (SEQ ID NO: 82), four consecutive G4S sequences (SEQ ID NO: 83), five consecutive G4S sequences (SEQ ID NO: 84), or six consecutive G4S sequences (SEQ ID NO: 85).

### 5.6. Hinge Domains

In certain embodiments, an Fc domain in a VEGF antagonist of the disclosure comprises a hinge region, e.g., a hinge region composed of two hinge domains. A hinge domain can be used to connect a VEGF binding domain to an Fc domain or to stabilize the configuration of the hexameric VEGF antagonist or its Fc core.

The hinge region can comprise a native or a modified hinge domain. The term “hinge domain”, unless the context dictates otherwise, refers to a naturally or non-naturally occurring hinge sequence that in the context of a single or monomeric polypeptide chain is a monomeric hinge domain. Sometimes, in the context of a dimeric polypeptide, two associated hinge sequences on separate polypeptide chains are referred to as a “hinge region”.

A native hinge region is the hinge region that would normally be found between Fab and Fc domains in a naturally occurring antibody. A modified hinge region is any hinge that differs in length and/or composition from the native hinge region. Such hinges can include hinge regions from other species, such as human, mouse, rat, rabbit, shark, pig, hamster, camel, llama or goat hinge regions. Other modified hinge regions may comprise a complete hinge region derived from an antibody of a different class or subclass from that of the heavy chain Fc domain or Fc region. Alternatively, the modified hinge region may comprise part of a natural hinge or a repeating unit in which each unit in the repeat is derived from a natural hinge region. In a further alternative, the natural hinge region may be altered by converting one or more cysteine or other residues into neutral residues, such as serine or alanine, or by converting suitably placed residues into cysteine residues. By such means the number of cysteine residues in the hinge region may be increased or decreased. Other modified hinge regions may be entirely synthetic and may be designed to possess desired properties such as length, cysteine composition and flexibility.

A number of modified hinge regions have already been described for example, in U.S. Pat. No. 5,677,425, WO 99/15549, WO 2005/003170, WO 2005/003169, WO 2005/003170, WO 98/25971 and WO 2005/003171 and these are incorporated herein by reference.

In various embodiments, positions 233-236 within a hinge domain may be G, G, G and unoccupied; G, G, unoccupied, and unoccupied; G, unoccupied, unoccupied, and unoccupied; or all unoccupied, with positions numbered by EU numbering.

In some embodiments, an Fc domain of the disclosure comprises a modified hinge domain that reduces binding affinity for an Fcγ receptor relative to a wild-type hinge domain of the same isotype (e.g., human IgG1 or human IgG4).

In one embodiment, the Fc domain of one or both chains of an Fc unit possesses an intact hinge domain at its N-terminus.

In one embodiment both the Fc domain and the hinge region of an Fc domain of the disclosure are derived from IgG4 and the hinge region comprises the modified sequence CPPC (SEQ ID NO: 75). The core hinge region of human IgG4 contains the sequence CPSC (SEQ ID NO: 86) compared to IgG1 that contains the sequence CPPC (SEQ ID NO: 75). The serine residue present in the IgG4 sequence leads to increased flexibility in this region, and therefore a proportion of molecules form disulfide bonds within the same protein chain (an intrachain disulfide) rather than bridging to the other heavy chain in the IgG molecule to form the interchain disulfide (Angel et al., 1993, Mol Immunol 30(1): 105-108). Changing the serine residue to a proline to give the same core sequence as IgG1 allows complete formation of inter-chain disulfides in the IgG4 hinge region, thus reducing heterogeneity in the purified product. This altered isotype is termed IgG4P.

Additional modified hinge regions contemplated herein include those having a truncated hinge domain which is missing the first one, two, three, four, or five amino acids relative to the wild type immunoglobulin sequence (e.g., wild type IgG1, IgG2, IgG3, or IgG4). For example, in some embodiments, an Fc domain of a VEGF antagonist of the disclosure comprises an IgG1 Fc domain having a truncated hinge domain which is missing the first one, two, three, four, or five amino acids relative to wild type IgG1. In some embodiments, the truncated hinge domain is missing the first five amino acids, EPKSC (SEQ ID NO: 80), relative to wild type IgG1. Examples of multimerization domains comprising IgG1 Fc domains having such a truncated hinge domain include, for example, those presented in SEQ ID NOS: 5, 51, 7, 33, 8, 9, 10, and 11. Fc domains having such truncated hinge domains may be particularly used in VEGF antagonists comprising a VEGF binding domain which is or comprises one or more VEGF receptor domains, such as a VEGF binding domain as described in Section 5.3.2, which is N-terminal to the Fc domain. In some embodiments, there is no intervening sequence between the VEGF binding domain and the Fc domain.

### 5.6.1. Chimeric Hinge Sequences

The hinge region can be a chimeric hinge region, with or without an N-terminal truncation as described herein, e.g., a truncated hinge domain missing the first five amino acids, EPKSC (SEQ ID NO: 80), relative to wild type IgG1.

For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region.

In particular embodiments, a chimeric hinge region comprises the amino acid sequence EPKSCDKTHTCPPCPAPPVA (SEQ ID NO:100 herein; previously disclosed as SEQ ID NO:8 of WO2014/121087, which is incorporated by reference in its entirety herein), ESKYGPPCPPCPAPPVA (SEQ ID NO:101 herein; previously disclosed as SEQ ID NO:9 of WO2014/121087), or a chimeric hinge sequence in which up to five amino acids are truncated from the N-terminus of the amino acid sequence of SEQ ID NO: 100 or SEQ ID NO: 101. Such chimeric hinge sequences can be suitably linked to an IgG4 CH2 region (for example by incorporation into an IgG4 Fc domain, for example a human or murine Fc domain, which can be further modified in the CH2 and/or CH3 domain to reduce effector function, for example as described below.

5.6.2. Hinge Sequences with Reduced Effector Function

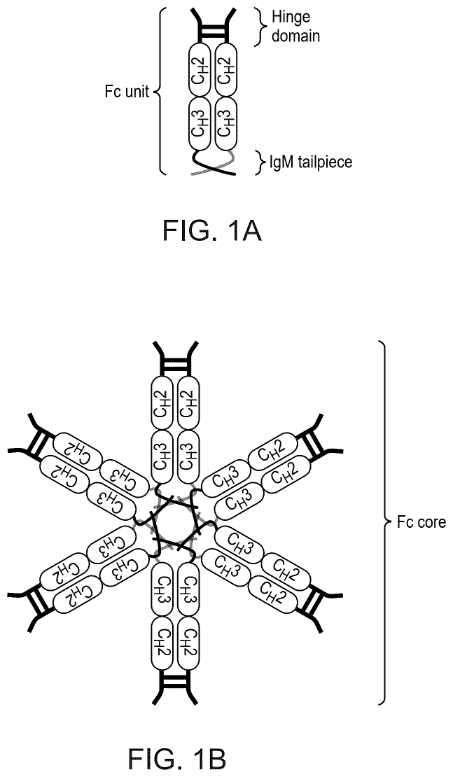

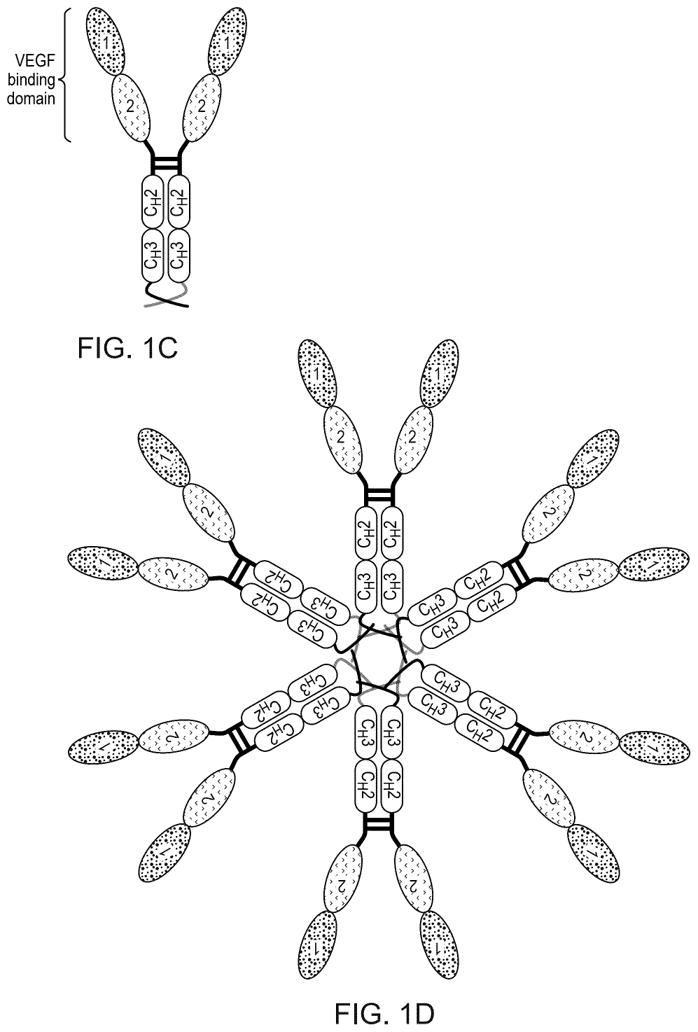

In further embodiments, the hinge region can be modified to reduce effector function, for example as described in WO2016161010A2, which is incorporated by reference in its entirety herein. In various embodiments, the positions 233-236 of the modified hinge region are G, G, G and unoccupied; G, G, unoccupied, and unoccupied; G, unoccupied, unoccupied, and unoccupied; or all unoccupied, with positions numbered by EU numbering (as shown in FIG. 1 of WO2016161010A2). These segments can be represented as GGG-, GG--, G--- or --- with “-” representing an unoccupied position.

Position 236 is unoccupied in canonical human IgG2 but is occupied by in other canonical human IgG isotypes. Positions 233-235 are occupied by residues other than G in all four human isotypes (as shown in FIG. 1 of WO2016161010A2).

The hinge modification within positions 233-236 can be combined with position 228 being occupied by P. Position 228 is naturally occupied by P in human IgG1 and IgG2 but is occupied by S in human IgG4 and R in human IgG3. An S228P mutation in an IgG4 antibody is advantageous in stabilizing an IgG4 antibody and reducing exchange of heavy chain light chain pairs between exogenous and endogenous antibodies. Preferably positions 226-229 are occupied by C, P, P and C respectively.

Exemplary hinge regions have residues 226-236, sometimes referred to as middle (or core) and lower hinge, occupied by the modified hinge sequences designated GGG-(233-236), GG--(233-236), G---(233-236) and no G (233-236). Optionally, the hinge domain amino acid sequence comprises CPPCPAPGGG-GPSVF (SEQ ID NO: 87) (previously disclosed as SEQ ID NO: 1 of WO2016161010A2), CPPCPAPGG--GPSVF (SEQ ID NO: 88) (previously disclosed as SEQ ID NO:2 of WO2016161010A2), CPPCPAPG---GPSVF (SEQ ID NO: 89) (previously disclosed as SEQ ID NO:3 of WO2016161010A2), or CPPCPAP---GPSVF (SEQ ID NO: 90) (previously disclosed as SEQ ID NO:4 of WO2016161010A2).

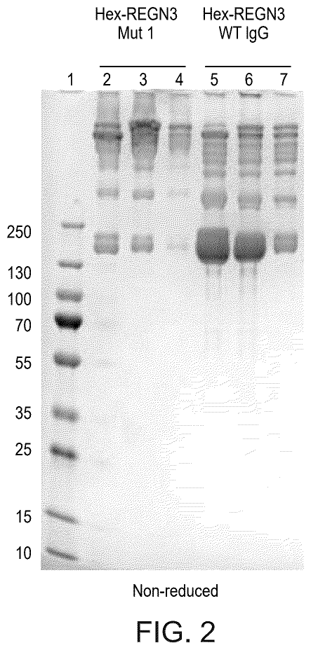

The modified hinge regions described above can be incorporated into a heavy chain constant region, which typically include CH2 and CH3 domains, and which may have an additional hinge segment (e.g., an upper hinge) flanking the designated region. Such additional constant region segments present are typically of the same isotype, preferably a human isotype, although can be hybrids of different isotypes. The isotype of such additional human constant regions segments is preferably human IgG4 but can also be human IgG1, IgG2, or IgG3 or hybrids thereof in which domains are of different isotypes. Exemplary sequences of human IgG1, IgG2 and IgG4 are shown in FIGS. 2-4 of WO2016161010A2.

In specific embodiments, the modified hinge sequences can be linked to an IgG4 CH2 region (for example by incorporation into an IgG4 Fc domain, for example a human or murine Fc domain, which can be further modified in the CH2 and/or CH3 domain to reduce effector function).

### 5.7. Nucleic Acids, Host Cells and Methods of Production

In another aspect, the disclosure provides nucleic acids encoding VEGF antagonists of the disclosure.

In some embodiments, a VEGF antagonist is encoded by a single nucleic acid. In other embodiments, a VEGF antagonist can be encoded by a plurality (e.g., two, three, four or more) nucleic acids.

A single nucleic acid can encode an entire VEGF antagonist or a portion of a VEGF antagonist (for example, a single nucleic acid can encode two polypeptide chains of a VEGF antagonist comprising three, four or more polypeptide chains, or three polypeptide chains of a VEGF antagonist comprising four or more polypeptide chains).

For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements and separated by internal ribosome entry site (IRES) sequences allowing for translation into separate polypeptides.

In some embodiments, a VEGF antagonist comprising two or more polypeptide chains is encoded by two or more nucleic acids. The number of nucleic acids encoding a VEGF antagonist can be equal to or less than the number of polypeptide chains in the VEGF antagonist (for example, when two or more polypeptide chains are encoded by a single nucleic acid).

The nucleic acids of the disclosure can be DNA or RNA (e.g., mRNA).

In another aspect, the disclosure provides host cells and vectors containing the nucleic acids of the disclosure. The nucleic acids may be present in a single vector or separate vectors present in the same host cell or separate host cell, as described in more detail herein below.

VEGF antagonists of the disclosure can be produced by culturing a host cell (e.g., as described in Section 5.7.2) under conditions suitable for expression of the VEGF antagonist, and recovering the VEGF antagonist from the host cell (or host cell culture medium).

### 5.7.1. Vectors

The disclosure provides vectors comprising nucleotide sequences encoding a VEGF antagonist or a component thereof described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes can be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed or introduced into the same cell by co-transformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid-based transfection, or other conventional techniques. Methods and conditions for culturing the resulting transfected cells and for recovering the expressed polypeptides are known to those skilled in the art and may be varied or optimized depending upon the specific expression vector and mammalian host cell employed, based upon the present description.

### 5.7.2. Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, Hela cells, COS cells, CHO cells, HEK293 cells, BHK cells and MDCKII cells. Suitable insect cells include, but are not limited to, Sf9 cells.

### 5.8. Pharmaceutical Compositions

The VEGF antagonists of the disclosure may be in the form of compositions comprising the VEGF antagonist and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the VEGF antagonists and, for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravitreally, ocularly, intraocularly, subconjunctivally, intravenously, intramuscularly, intratumorally, intrathecally, topically, or locally. The most suitable route for administration in any given case will depend on the particular antibody, the subject, and the nature and severity of the disease and the physical condition of the subject. In some embodiments, the pharmaceutical composition is administered by ocular, intraocular, intravitreal or subconjunctival injection. In some embodiments, the pharmaceutical composition is administered intravitreally. In other embodiments, the VEGF antagonist can be administered by topical administration, e.g., via eye drops or other liquid, gel, ointment, or fluid which contains the VEGF antagonist and can be applied directly to the eye.

Pharmaceutical compositions can be conveniently presented in unit dosage forms containing a predetermined amount of a VEGF antagonist of the disclosure per dose. The quantity of a VEGF antagonist included in a unit dose will depend on the disease being treated, as well as other factors as are well known in the art. Such unit dosages may be in the form of a lyophilized dry powder containing an amount of VEGF antagonist suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of VEGF antagonist suitable for a single administration.

The pharmaceutical compositions may also be supplied in bulk from containing quantities of VEGF antagonists suitable for multiple administrations.

Pharmaceutical compositions may be prepared for storage as lyophilized formulations or aqueous solutions by mixing a VEGF antagonist having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives should be nontoxic to the recipients at the dosages and concentrations employed.

Buffering agents help to maintain the pH in the range which approximates physiological conditions. They may be present at a wide variety of concentrations but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol, and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10 wt % per wt of VEGF antagonist.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188, etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

The VEGF antagonists of the disclosure can be formulated as pharmaceutical compositions comprising the VEGF antagonists, for example containing one or more pharmaceutically acceptable excipients or carriers. To prepare pharmaceutical or sterile compositions comprising the VEGF antagonists of the present disclosure, a VEGF antagonist preparation can be combined with one or more pharmaceutically acceptable excipient or carrier.

For example, formulations of VEGF antagonists can be prepared by mixing VEGF antagonists with physiologically acceptable carriers, excipients, or stabilizers in the form of, e.g., lyophilized powders, slurries, aqueous solutions, lotions, or suspensions (see, e.g., Hardman et al., 2001, Goodman and Gilman's The Pharmacological Basis of Therapeutics, McGraw-Hill, New York, N.Y.; Gennaro, 2000, Remington: The Science and Practice of Pharmacy, Lippincott, Williams, and Wilkins, New York, N.Y.; Avis, et al. (eds.), 1993, Pharmaceutical Dosage Forms: General Medications, Marcel Dekker, NY; Lieberman, et al. (eds.), 1990, Pharmaceutical Dosage Forms: Tablets, Marcel Dekker, NY; Lieberman, et al. (eds.), 1990, Pharmaceutical Dosage Forms: Disperse Systems, Marcel Dekker, NY; Weiner and Kotkoskie, 2000, Excipient Toxicity and Safety, Marcel Dekker, Inc., New York, N.Y.).

### 5.9. Methods of Using VEGF Antagonists

The present disclosure provides methods for using and applications for VEGF antagonists of the disclosure.

Avidity of immunoglobulins reflects the strength of the interactions between the immunoglobulin and its target. Hexameric VEGF antagonists of the disclosure provide high avidity relative to standard IgG molecules, which are bivalent as they can only be linked to two target binding domains. In contrast, the avidity of the VEGF antagonists of the disclosure is similar to the avidity of IgM molecules. For instance, a hexameric VEGF antagonist is capable of being bound to twelve copies of VEGF.

High avidity immunoglobulins, such as pentameric or hexameric IgM molecules have low stability and relatively short half-lives. The hexameric VEGF antagonists of the disclosure can provide increased stability relative to IgM molecules. As a result, high stability of hexameric VEGF antagonists results in improved shelf-lives and longer half-lives, reducing the need for frequent application of the pharmaceutical compositions comprising hexameric VEGF antagonists.

VEGF antagonists of the disclosure can be used to treat diseases and disorders including eye diseases and other conditions involving excessive, unwanted, or inappropriate angiogenesis. In some embodiments, VEGF antagonists are used in a subject to treat an angiogenic eye disorder, e.g., age-related macular degeneration (e.g., wet AMD, exudative AMD, etc.), retinal vein occlusion (RVO), central retinal vein occlusion (CRVO; e.g., macular edema following CRVO), branch retinal vein occlusion (BRVO), diabetic macular edema (DME), choroidal neovascularization (CNV; e.g., myopic CNV), iris neovascularization, neovascular glaucoma, post-surgical fibrosis in glaucoma, proliferative vitreoretinopathy (PVR), optic disc neovascularization, corneal neovascularization, retinal neovascularization, vitreal neovascularization, pannus, pterygium, vascular retinopathy, diabetic retinopathies, and edemas associated with injuries or inflammation.

In some embodiments, the angiogenic eye disorder is diabetic macular edema. In some embodiments, the angiogenic eye disorder is central retinal vein occlusion. In some embodiments, the angiogenic eye disorder is branch retinal vein occlusion. In some embodiments, the angiogenic eye disorder is corneal neovascularization.

The methods of the disclosure can comprise sequentially administering to a patient multiple doses of a VEGF antagonist. As used herein, “sequentially administering” means that each dose of VEGF antagonist is administered to the patient at a different point in time, e.g., on different days separated by a predetermined interval (e.g., hours, days, weeks or months). In some embodiments, the methods of the disclosure comprise sequentially administering to the patient a single initial dose of a VEGF antagonist, followed by one or more subsequent doses of the VEGF antagonist.

The methods of the disclosure may comprise administering to a subject any number of secondary doses of a VEGF antagonist. In some embodiments, only a single secondary dose is administered to the subject. In other embodiments, two or more (e.g., 2, 3, 4, 5, 6, 7, 8, or more) secondary doses are administered to the patient.

In some embodiments, the initial and subsequent doses contain the same amount of VEGF antagonist. In other embodiments, the initial and subsequent doses contain different amounts of VEGF antagonist.

The frequency of VEGF antagonist administration depends on the type and severity of the disease or disorder being treated, and may be adjusted during the course of treatment regimen. In some embodiments, a subsequent dose is administered 2 to 4 (e.g., 2, 2.5, 3, 3.5, or 4) weeks after the immediately preceding dose. In some embodiments, a subsequent dose is administered at least 8 (e.g., 8, 8.5, 9, 9.5, 10, 10.5, 11, 11.5, 12, 12.5, 13, 13.5, 14, or more) weeks after the immediately preceding dose. The term “the immediately preceding dose,” as used herein, means, in a sequence of multiple administrations, the dose of VEGF antagonist which is administered to a patient prior to the administration of the very next dose in the sequence with no intervening doses.

### 6. SEQUENCES

Certain sequences of the disclosure are provided in Table S below. Should any discrepancy or inconsistency exist between the sequences listed in Table S and those presented elsewhere in the specification, the sequences and associated sequence identifiers listed in Table S shall govern.

TABLE S
Sequences

| | | SEQ |
|---|---|---|
| Description | Sequence | ID NO |
| WT IgG1 Fc | EPKSCDKTHTCPPCPAPELLGGPSVFLFPPKPKDTLMI | 1 |
| | SRTPEVTCVVVDVSHEDPEVKFNWYVDGVEVHNAKT | |
| | KPREEQYNSTYRVVSVLTVLHQDWLNGKEYKCKVSN | |
| | KALPAPIEKTISKAKGQPREPQVYTLPPSRDELTKNQV | |
| | SLTCLVKGFYPSDIAVEWESNGQPENNYKTTPPVLDS | |
| | DGSFFLYSKLTVDKSRWQQGNVFSCSVMHEALHNHY | |
| | TQKSLSLSPGK | |
| Tailpiece | PTLYNVSLX₁MSDTX₂GTCY | 2 |
| | (where X₁ is any amino acid and X₂ is | |
| | any amino acid) | |
| Tailpiece-WT | PTLYNVSLVMSDTAGTCY | 3 |
| Tailpiece-mut | PTLYNVSLIMSDTGGTCY | 4 |
| IgG1-T- | DKTHTCPPCPAPELLGGPSVFLFPPKPKDTLMISRTPE | 5 |
| Hex(WT) | VTCVVVDVSHEDPEVKFNWYVDGVEVHNAKTKPREE | |
| | QYNSTYRVVSVLTVLHQDWLNGKEYKCKVSNKALPAP | |
| | IEKTISKAKGQPREPQVYTLPPSRDELTKNQVSLTCLV | |
| | KGFYPSDIAVEWESNGQPENNYKTTPPVLDSDGSFFL | |
| | YSKLTVDKSRWQQGNVFSCSVMHEALHNHYTQKSLD | |
| | KSTGKPTLYNVSLVMSDTAGTCY | |
| IgG1-T- | EPKSCDKTHTCPPCPAPELLGGPSVFLFPPKPKDTLMI | 6 |
| Hex(Mut) | SRTPEVTCVVVDVSHEDPEVKFNWYVDGVEVHNAKT | |
| | KPREEQYNSTYRVVSVLTVCHQDWLNGKEYKCKVSN | |
| | KALPAPIEKTISKAKGQPREPQVYTLPPSREEMTKNQV | |
| | SLTCLVKGFYPSDIAVEWESNGQPENNYKTTPPVLDS | |
| | DGSFFLYSKLTVDKSRWQQGNVFSCSVMHEALHNHY | |
| | TQKSLDKSTGKPTLYNVSLIMSDTGGTCY | |
| IgG1-T- | DKTHTCPPCPAPELLGGPSVFLFPPKPKDTLMISRTPE | 7 |
| Hex(Mut1) | VTCVVVDVSHEDPEVKFNWYVDGVEVHNAKTKPREE | |
| | QYNSTYRVVSVLTVCHQDWLNGKEYKCKVSNKALPA | |
| | PIEKTISKAKGQPREPQVYTLPPSREEMTKNQVSLTCL | |
| | VKGFYPSDIAVEWESNGQPENNYKTTPPVLDSDGSFF | |
| | LYSKLTVDKSRWQQGNVFSCSVMHEALHNHYTQKSL | |
| | DKSTGKPTLYNVSLIMSDTGGTCY | |
| IgG1-T- | DKTHTCPPCPAPELLGGPSVFLFPPKPKDTLMISRTPE | 33 |
| Hex(Mut1-V2) | VTCVVVDVSHEDPEVKFNWYVDGVEVHNAKTKPREE | |
| | QYNSTYRVVSVLTVCHQDWLNGKEYKCKVSNKALPA | |
| | PIEKTISKAKGQPREPQVYTLPPSREEMTKNQVSLTCL | |
| | VKGFYPSDIAVEWESNGQPENNYKTTPPVLDSDGSFF | |
| | LYSKLTVDKSRWQQGNVFSCSVMHEALHNHYTQKSL | |
| | DKSTGKPTLYNVSLVMSDTAGTCY | |
| IgG1-T- | DKTHTCPPCPAPELLGGPSVFLFPPKPKDTLMCSRTP | 8 |
| Hex(Mut2) | EVTCVVVDVSHEAPEVKFNWYVDGVEVHNAKTKPRE | |
| | EQYNSTYRVVSVLTVLHQDWLNGKEYKCKVSNKALPA | |
| | GIEKTISKAKGQPREPQVYTLPPSRDELTKNQVSLTCL | |
| | VKGFYPSDIAVEWESNGQPENNYKTTPPVLDSDGSFF | |
| | LYSKLTVDKSRWQQGNVFSCSVMHEALHNHYTQKSL | |
| | SLSPGKPTLYNVSLVMSDTAGTCY | |
| IgG1-T-Mut1- | DKTHTCPPCPAPEAAGGPSVFLFPPKPKDTLMISRTPE | 9 |
| V2 LALAPG | VTCVVVDVSHEDPEVKFNWYVDGVEVHNAKTKPREE | |
| | QYNSTYRVVSVLTVCHQDWLNGKEYKCKVSNKALGA | |
| | PIEKTISKAKGQPREPQVYTLPPSREEMTKNQVSLTCL | |
| | VKGFYPSDIAVEWESNGQPENNYKTTPPVLDSDGSFF | |
| | LYSKLTVDKSRWQQGNVFSCSVMHEALHNHYTQKSL | |
| | DKSTGKPTLYNVSLVMSDTAGTCY | |
| IgG1-T-Mut1- | DKTHTCPPCPAPPVAGPSVFLFPPKPKDTLMISRTPEV | 10 |
| V2 | TCVVVDVSHEDPEVKFNWYVDGVEVHNAKTKPREEQ | |
| PVA/P329A | YNSTYRVVSVLTVCHQDWLNGKEYKCKVSNKALAAPI | |
| | EKTISKAKGQPREPQVYTLPPSREEMTKNQVSLTCLV | |
| | KGFYPSDIAVEWESNGQPENNYKTTPPVLDSDGSFFL | |
| | YSKLTVDKSRWQQGNVFSCSVMHEALHNHYTQKSLD | |
| | KSTGKPTLYNVSLVMSDTAGTCY | |
| IgG1-T-Mut2 | DKTHTCPPCPAPEAAGGPSVFLFPPKPKDTLMCSRTP | 11 |
| LALAPG | EVTCVVVDVSHEAPEVKFNWYVDGVEVHNAKTKPRE | |
| | EQYNSTYRVVSVLTVLHQDWLNGKEYKCKVSNKALG | |
| | AGIEKTISKAKGQPREPQVYTLPPSRDELTKNQVSLTC | |
| | LVKGFYPSDIAVEWESNGQPENNYKTTPPVLDSDGSF | |
| | FLYSKLTVDKSRWQQGNVFSCSVMHEALHNHYTQKS | |
| | LSLSPGKPTLYNVSLVMSDTAGTCY | |
| IgG4/IgG1-T- | ESKYGPPCPPCPAPGGGGPSVFLFPPKPKDTLMISRT | 12 |
| Mut1 | PEVTCVVVDVSQEDPEVQFNWYVDGVEVHNAKTKPR | |
| | EEQFNSTYRVVSVLTVCHQDWLNGKEYKCKVSNKGL | |
| | PSSIEKTISKAKGQPREPQVYTLPPSREEMTKNQVSLT | |
| | CLVKGFYPSDIAVEWESNGQPENNYKTTPPVLDSDGS | |
| | FFLYSKLTVDKSRWQQGNVFSCSVMHEALHNHYTQK | |
| | SLDKSTGKPTLYNVSLVMSDTAGTCY | |
| VEGFR1 | GRELVIPCRVTSPNITVTLKKFPLDTLIPDGKRIIWDSRK | 13 |
| domain 2 (1) | GFIISNATYKEIGLLTCEATVNGH | |
| VEGFR1 | SDTGRPFVEMYSEIPEIIHMTEGRELVIPCRVTSPNITV | 14 |
| domain 2 (2) | TLKKFPLDTLIPDGKRIIWDSRKGFIISNATYKEIGLLTCE | |
| | ATVNGHLYKTNYLTHRQTNTIID | |
| VEGFR2 | VVLSPSHGIELSVGEKLVLNCTARTELNVGIDFNWEYP | 15 |
| domain 3 | SSKHQHKKLVNRDLKTQSGSEMKKFLSTLTIDGVTRS | |
| | DQGLYTCAASSGLMTKKNSTFVRVHEK | |
| VEGFR2 | VVLSPSHGIELSVGEKLVLNCTARTELNVGIDFNWEYP | 50 |
| domain 3 (2) | SSKHQHKKLVNRDLKTQSGSEMKKFLSTLTIDGVTRS | |
| | DQGLYTCAASSGLMTKKNST | |
| Example | SDTGRPFVEMYSEIPEIIHMTEGRELVIPCRVTSPNITV | 16 |
| VEGF binding | TLKKFPLDTLIPDGKRIIWDSRKGFIISNATYKEIGLLTCE | |
| domain | ATVNGHLYKTNYLTHRQTNTIIDVVLSPSHGIELSVGEK | |
| | LVLNCTARTELNVGIDFNWEYPSSKHQHKKLVNRDLK | |
| | TQSGSEMKKFLSTLTIDGVTRSDQGLYTCAASSGLMT | |
| | KKNSTFVRVHEK | |
| Hex-REGN3 | SDTGRPFVEMYSEIPEIIHMTEGRELVIPCRVTSPNITV | 17 |
| (IgG1) | TLKKFPLDTLIPDGKRIIWDSRKGFIISNATYKEIGLLTCE | |
| Mut1-V2 | ATVNGHLYKTNYLTHRQTNTIIDVVLSPSHGIELSVGEK | |
| LALAPG | LVLNCTARTELNVGIDFNWEYPSSKHQHKKLVNRDLK | |
| | TQSGSEMKKFLSTLTIDGVTRSDQGLYTCAASSGLMT | |
| | KKNSTFVRVHEKDKTHTCPPCPAPEAAGGPSVFLFPP | |
| | KPKDTLMISRTPEVTCVVVDVSHEDPEVKFNWYVDGV | |
| | EVHNAKTKPREEQYNSTYRVVSVLTVCHQDWLNGKE | |
| | YKCKVSNKALGAPIEKTISKAKGQPREPQVYTLPPSRE | |
| | EMTKNQVSLTCLVKGFYPSDIAVEWESNGQPENNYKT | |
| | TPPVLDSDGSFFLYSKLTVDKSRWQQGNVFSCSVMH | |
| | EALHNHYTQKSLDKSTGKPTLYNVSLVMSDTAGTCY | |
| Hex-REGN3 | SDTGRPFVEMYSEIPEIIHMTEGRELVIPCRVTSPNITV | 18 |
| (IgG1) Mut1- | TLKKFPLDTLIPDGKRIIWDSRKGFIISNATYKEIGLLTCE | |
| V2 | ATVNGHLYKTNYLTHRQTNTIIDVVLSPSHGIELSVGEK | |
| PVA/P329A | LVLNCTARTELNVGIDFNWEYPSSKHQHKKLVNRDLK | |
| | TQSGSEMKKFLSTLTIDGVTRSDQGLYTCAASSGLMT | |
| | KKNSTFVRVHEKDKTHTCPPCPAPPVAGPSVFLFPPK | |
| | PKDTLMISRTPEVTCVVVDVSHEDPEVKFNWYVDGVE | |
| | VHNAKTKPREEQYNSTYRVVSVLTVCHQDWLNGKEY | |
| | KCKVSNKALAAPIEKTISKAKGQPREPQVYTLPPSREE | |
| | MTKNQVSLTCLVKGFYPSDIAVEWESNGQPENNYKTT | |
| | PPVLDSDGSFFLYSKLTVDKSRWQQGNVFSCSVMHE | |
| | ALHNHYTQKSLDKSTGKPTLYNVSLVMSDTAGTCY | |
| Hex-REGN3 | SDTGRPFVEMYSEIPEIIHMTEGRELVIPCRVTSPNITV | 19 |
| (IgG1) Mut2 | TLKKFPLDTLIPDGKRIIWDSRKGFIISNATYKEIGLLTCE | |
| LALAPG | ATVNGHLYKTNYLTHRQTNTIIDVVLSPSHGIELSVGEK | |
| | LVLNCTARTELNVGIDFNWEYPSSKHQHKKLVNRDLK | |
| | TQSGSEMKKFLSTLTIDGVTRSDQGLYTCAASSGLMT | |
| | KKNSTFVRVHEKDKTHTCPPCPAPEAAGGPSVFLFPP | |
| | KPKDTLMCSRTPEVTCVVVDVSHEAPEVKFNWYVDG | |
| | VEVHNAKTKPREEQYNSTYRVVSVLTVLHQDWLNGK | |
| | EYKCKVSNKALGAGIEKTISKAKGQPREPQVYTLPPSR | |
| | DELTKNQVSLTCLVKGFYPSDIAVEWESNGQPENNYK | |
| | TTPPVLDSDGSFFLYSKLTVDKSRWQQGNVFSCSVM | |
| | HEALHNHYTQKSLSLSPGKPTLYNVSLVMSDTAGTCY | |
| Hex-REGN3 | SDTGRPFVEMYSEIPEIIHMTEGRELVIPCRVTSPNITV | 20 |
| (IgG4/IgG1 | TLKKFPLDTLIPDGKRIIWDSRKGFIISNATYKEIGLLTCE | |
| chimera) Mut1- | ATVNGHLYKTNYLTHRQTNTIIDVVLSPSHGIELSVGEK | |
| V2 | LVLNCTARTELNVGIDFNWEYPSSKHQHKKLVNRDLK | |
| | TQSGSEMKKFLSTLTIDGVTRSDQGLYTCAASSGLMT | |
| | KKNSTFVRVHEKESKYGPPCPPCPAPGGGGPSVFLFP | |
| | PKPKDTLMISRTPEVTCVVVDVSQEDPEVQFNWYVDG | |
| | VEVHNAKTKPREEQFNSTYRVVSVLTVCHQDWLNGK | |
| | EYKCKVSNKGLPSSIEKTISKAKGQPREPQVYTLPPSR | |
| | EEMTKNQVSLTCLVKGFYPSDIAVEWESNGQPENNYK | |
| | TTPPVLDSDGSFFLYSKLTVDKSRWQQGNVFSCSVM | |
| | HEALHNHYTQKSLDKSTGKPTLYNVSLVMSDTAGTCY | |
| hIgG2 Fc | ERKCCVECPPCPAPPVAGPSVFLFPPKPKDTLMISRTP | 21 |
| | EVTCVVVDVSHEDPEVQFNWYVDGVEVHNAKTKPRE | |
| | EQFNSTFRVVSVLTVVHQDWLNGKEYKCKVSNKGLP | |
| | APIEKTISKTKGQPREPQVYTLPPSREEMTKNQVSLTC | |
| | LVKGFYPSDISVEWESNGQPENNYKTTPPMLDSDGSF | |
| | FLYSKLTVDKSRWQQGNVFSCSVMHEALHNHYTQKS | |
| | LSLSPGK | |
| hIgG3 Fc | ELKTPLGDTTHTCPRCPEPKSCDTPPPCPRCPEPKSC | 22 |
| | DTPPPCPRCPEPKSCDTPPPCPRCPAPELLGGPSVFL | |
| | FPPKPKDTLMISRTPEVTCVVVDVSHEDPEVQFKWYV | |
| | DGVEVHNAKTKPREEQYNSTFRVVSVLTVLHQDWLN | |
| | GKEYKCKVSNKALPAPIEKTISKTKGQPREPQVYTLPP | |
| | SREEMTKNQVSLTCLVKGFYPSDIAVEWESSGQPENN | |
| | YNTTPPMLDSDGSFFLYSKLTVDKSRWQQGNIFSCSV | |
| | MHEALHNRFTQKSLSLSPGK | |
| hIgG4 Fc | ESKYGPPCPSCPAPEFLGGPSVFLFPPKPKDTLMISRT | 23 |
| | PEVTCVVVDVSQEDPEVQFNWYVDGVEVHNAKTKPR | |
| | EEQFNSTYRVVSVLTVLHQDWLNGKEYKCKVSNKGLP | |
| | SSIEKTISKAKGQPREPQVYTLPPSQEEMTKNQVSLTC | |
| | LVKGFYPSDIAVEWESNGQPENNYKTTPPVLDSDGSF | |
| | FLYSRLTVDKSRWQEGNVFSCSVMHEALHNHYTQKS | |
| | LSLSLGK | |
| hIgG4s Fc | ESKYGPPCPPCPAPPVAGPSVFLFPPKPKDTLMISRTP | 24 |
| | EVTCVVVDVSQEDPEVQFNWYVDGVEVHNAKTKPRE | |
| | EQFNSTYRVVSVLTVLHQDWLNGKEYKCKVSNKGLPS | |
| | SIEKTISKAKGQPREPQVYTLPPSQEEMTKNQVSLTCL | |
| | VKGFYPSDIAVEWESNGQPENNYKTTPPVLDSDGSFF | |
| | LYSRLTVDKSRWQEGNVFSCSVMHEALHNHYTQKSL | |
| | SLSLGK | |
| hIgG1 PVA Fc | EPKSCDKTHTCPPCPAPPVAGPSVFLFPPKPKDTLMIS | 25 |
| | RTPEVTCVVVDVSHEDPEVKFNWYVDGVEVHNAKTK | |
| | PREEQYNSTYRVVSVLTVLHQDWLNGKEYKCKVSNK | |
| | ALPAPIEKTISKAKGQPREPQVYTLPPSRDELTKNQVS | |
| | LTCLVKGFYPSDIAVEWESNGQPENNYKTTPPVLDSD | |
| | GSFFLYSKLTVDKSRWQQGNVFSCSVMHEALHNHYT | |
| | QKSLSLSPGK | |
| hIgG1 | EPKSCDKTHTCPPCPAPPVAGPSVFLFPPKPKDTLMIS | 26 |
| PVA/P329A Fc | RTPEVTCVVVDVSHEDPEVKFNWYVDGVEVHNAKTK | |
| | PREEQYNSTYRVVSVLTVLHQDWLNGKEYKCKVSNK | |
| | ALAAPIEKTISKAKGQPREPQVYTLPPSRDELTKNQVS | |
| | LTCLVKGFYPSDIAVEWESNGQPENNYKTTPPVLDSD | |
| | GSFFLYSKLTVDKSRWQQGNVFSCSVMHEALHNHYT | |
| | QKSLSLSPGK | |
| hIgG1 | EPKSCDKTHTCPPCPAPEAAGGPSVFLFPPKPKDTLMI | 27 |
| LALAPG Fc | SRTPEVTCVVVDVSHEDPEVKFNWYVDGVEVHNAKT | |
| | KPREEQYNSTYRVVSVLTVLHQDWLNGKEYKCKVSN | |
| | KALGAPIEKTISKAKGQPREPQVYTLPPSRDELTKNQV | |
| | SLTCLVKGFYPSDIAVEWESNGQPENNYKTTPPVLDS | |
| | DGSFFLYSKLTVDKSRWQQGNVFSCSVMHEALHNHY | |
| | TQKSLSLSPGK | |
| hIgG1 PVA | EPKSCDKTHTCPPCPAPPVAGPSVFLFPPKPKDTLMIS | 28 |
| star | RTPEVTCVVVDVSHEDPEVKFNWYVDGVEVHNAKTK | |
| | PREEQYNSTYRVVSVLTVLHQDWLNGKEYKCKVSNK | |
| | ALPAPIEKTISKAKGQPREPQVYTLPPSRDELTKNQVS | |
| | LTCLVKGFYPSDIAVEWESNGQPENNYKTTPPVLDSD | |
| | GSFFLYSKLTVDKSRWQQGNVFSCSVMHEALHNRFT | |
| | QKSLSLSPGK | |
| hIgG1s | DKKVEPKSCDKTHTCPPCPAPPVAGPSVFLFPPKPKD | 29 |
| | TLMISRTPEVTCVVVDVSQEDPEVQFNWYVDGVEVHN | |
| | AKTKPREEQFNSTYRVVSVLTVLHQDWLNGKEYKCKV | |
| | SNKGLPSSIEKTISKAKGQPREPQVYTLPPSRDELTKN | |
| | QVSLTCLVKGFYPSDIAVEWESNGQPENNYKTTPPVL | |
| | DSDGSFFLYSKLTVDKSRWQQGNVFSCSVMHEALHN | |
| | HYTQKSLSLSPGK | |
| hIgG1 N180G, | EPKSCDKTHTCPPCPAPELLGGPSVFLFPPKPKDTLMI | 30 |
| also referred to | SRTPEVTCVVVDVSHEDPEVKFNWYVDGVEVHNAKT | |
| as N297G | KPREEQYGSTYRVVSVLTVLHQDWLNGKEYKCKVSN | |
| | KALPAPIEKTISKAKGQPREPQVYTLPPSRDELTKNQV | |
| | SLTCLVKGFYPSDIAVEWESNGQPENNYKTTPPVLDS | |
| | DGSFFLYSKLTVDKSRWQQGNVFSCSVMHEALHNHY | |
| | TQKSLSLSPGK | |
| hIgG2 variant | ERKCCVECPPCPAPPVAGPSVFLFPPKPKDTLMISRTP | 31 |
| | EVTCVVVDVSHEDPEVQFNWYVDGVEVHNAKTKPRE | |
| | EQFNSTFRVVSVLTVVHQDWLNGKEYKCKVSNKGLP | |
| | APIEKTISKTKGQPREPQVYTLPPSREEMTKNQVSLTC | |
| | LVKGFYPSDIAVEWESNGQPENNYKTTPPMLDSDGSF | |
| | FLYSKLTVDKSRWQQGNVFSCSVMHEALHNHYTQKS | |
| | LSLSPGK | |
| hIgG4 S108P | ESKYGPPCPPCPAPEFLGGPSVFLFPPKPKDTLMISRT | 32 |
| | PEVTCVVVDVSQEDPEVQFNWYVDGVEVHNAKTKPR | |
| | EEQFNSTYRVVSVLTVLHQDWLNGKEYKCKVSNKGLP | |
| | SSIEKTISKAKGQPREPQVYTLPPSQEEMTKNQVSLTC | |
| | LVKGFYPSDIAVEWESNGQPENNYKTTPPVLDSDGSF | |
| | FLYSRLTVDKSRWQEGNVFSCSVMHEALHNHYTQKS | |
| | LSLSLGK | |
| HEX-REGN3 | SDTGRPFVEMYSEIPEIIHMTEGRELVIPCRVTSPNITV | 34 |
| (IgG4us) WT | TLKKFPLDTLIPDGKRIIWDSRKGFIISNATYKEIGLLTCE | |
| | ATVNGHLYKTNYLTHRQTNTIIDVVLSPSHGIELSVGEK | |
| | LVLNCTARTELNVGIDFNWEYPSSKHQHKKLVNRDLK | |
| | TQSGSEMKKFLSTLTIDGVTRSDQGLYTCAASSGLMT | |
| | KKNSTFVRVHESKYGPPCPPCPAPGGGGPSVFLFPPK | |
| | PKDTLMISRTPEVTCVVVDVSQEDPEVQFNWYVDGVE | |
| | VHNAKTKPREEQFNSTYRVVSVLTVLHQDWLNGKEYK | |
| | CKVSNKGLPSSIEKTISKAKGQPREPQVYTLPPSQEEM | |
| | TKNQVSLTCLVKGFYPSDIAVEWESNGQPENNYKTTP | |
| | PVLDSDGSFFLYSRLTVDKSRWQEGNVFSCSVMHEA | |
| | LHNHYTQKSLSLSLGKPTLYNVSLVMSDTAGTCY | |
| HEX-REGN3 | SDTGRPFVEMYSEIPEIIHMTEGRELVIPCRVTSPNITV | 35 |
| (IgG4us) Mut1 | TLKKFPLDTLIPDGKRIIWDSRKGFIISNATYKEIGLLTCE | |
| | ATVNGHLYKTNYLTHRQTNTIIDVVLSPSHGIELSVGEK | |
| | LVLNCTARTELNVGIDFNWEYPSSKHQHKKLVNRDLK | |
| | TQSGSEMKKFLSTLTIDGVTRSDQGLYTCAASSGLMT | |
| | KKNSTFVRVHESKYGPPCPPCPAPGGGGPSVFLFPPK | |
| | PKDTLMISRTPEVTCVVVDVSQEDPEVQFNWYVDGVE | |
| | VHNAKTKPREEQFNSTYRVVSVLTVCHQDWLNGKEY | |
| | KCKVSNKGLPSSIEKTISKAKGQPREPQVYTLPPSQEE | |
| | MTKNQVSLTCLVKGFYPSDIAVEWESNGQPENNYKTT | |
| | PPVLDSDGSFFLYSRLTVDKSRWQEGNVFSCSVMHE | |
| | ALHNHYTQKSLDKSTGKPTLYNVSLIMSDTGGTCY | |
| HEX-REGN3 | SDTGRPFVEMYSEIPEIIHMTEGRELVIPCRVTSPNITV | 36 |
| (IgG1) | TLKKFPLDTLIPDGKRIIWDSRKGFIISNATYKEIGLLTCE | |
| LALAPG WT | ATVNGHLYKTNYLTHRQTNTIIDVVLSPSHGIELSVGEK | |
| | LVLNCTARTELNVGIDFNWEYPSSKHQHKKLVNRDLK | |
| | TQSGSEMKKFLSTLTIDGVTRSDQGLYTCAASSGLMT | |
| | KKNSTFVRVHEKDKTHTCPPCPAPEAAGGPSVFLFPP | |
| | KPKDTLMISRTPEVTCVVVDVSHEDPEVKFNWYVDGV | |
| | EVHNAKTKPREEQYNSTYRVVSVLTVLHQDWLNGKE | |
| | YKCKVSNKALGAPIEKTISKAKGQPREPQVYTLPPSRD | |
| | ELTKNQVSLTCLVKGFYPSDIAVEWESNGQPENNYKT | |
| | TPPVLDSDGSFFLYSKLTVDKSRWQQGNVFSCSVMH | |
| | EALHNHYTQKSLSLSPGKPTLYNVSLVMSDTAGTCY | |
| HEX-REGN3 | SDTGRPFVEMYSEIPEIIHMTEGRELVIPCRVTSPNITV | 37 |
| (IgG1) | TLKKFPLDTLIPDGKRIIWDSRKGFIISNATYKEIGLLTCE | |
| LALAPG Mut1 | ATVNGHLYKTNYLTHRQTNTIIDVVLSPSHGIELSVGEK | |
| | LVLNCTARTELNVGIDFNWEYPSSKHQHKKLVNRDLK | |
| | TQSGSEMKKFLSTLTIDGVTRSDQGLYTCAASSGLMT | |
| | KKNSTFVRVHEKDKTHTCPPCPAPEAAGGPSVFLFPP | |
| | KPKDTLMISRTPEVTCVVVDVSHEDPEVKFNWYVDGV | |
| | EVHNAKTKPREEQYNSTYRVVSVLTVCHQDWLNGKE | |
| | YKCKVSNKALGAPIEKTISKAKGQPREPQVYTLPPSRE | |
| | EMTKNQVSLTCLVKGFYPSDIAVEWESNGQPENNYKT | |
| | TPPVLDSDGSFFLYSKLTVDKSRWQQGNVFSCSVMH | |
| | EALHNHYTQKSLDKSTGKPTLYNVSLIMSDTGGTCY | |

### 7. SPECIFIC EMBODIMENTS

While various specific embodiments have been illustrated and described, it will be appreciated that various changes can be made without departing from the spirit and scope of the disclosure(s). The present disclosure is exemplified by the numbered embodiments set forth below.

1. A VEGF antagonist comprising five or more dimers, each dimer comprising two polypeptide chains, each polypeptide chain comprising, in N- to C-terminal orientation:
(a) a VEGF binding domain comprising, which is optionally (I) a VEGF binding portion of a VEGF antibody or (II) a VEGF binding portion of a VEGF receptor, the VEGF binding portion of the VEGF receptor:
   (i) an Ig-like domain 2 of VEGFR1 comprising an amino acid sequence having at least 90% sequence identity (e.g., at least at least 91%, at least 92%, at least 93%, at least 94%, or at least 95% sequence identity) to SEQ ID NO: 13; and
   (ii) an Ig-like domain 3 of VEGFR2 comprising an amino acid sequence having at least 90% sequence identity (e.g., at least at least 91%, at least 92%, at least 93%, at least 94%, or at least 95% sequence identity) to SEQ ID NO: 15 or SEQ ID NO:50; and
(b) a multimerization domain comprising, in N- to C-terminal orientation:
   (i) an IgG Fc domain having at least 80%, optionally at least 90% sequence identity to the amino acid sequence of SEQ ID NO:1; and
   (ii) an IgM tailpiece having at least 80%, optionally at least 90%, sequence identity to the amino acid sequence of SEQ ID NO:2.

2. The VEGF antagonist of embodiment 1, wherein the IgM tailpiece has at least 85% sequence identity to the amino acid sequence of SEQ ID NO:2.

3 The VEGF antagonist of embodiment 1, wherein the IgM tailpiece has at least 90% sequence identity to the amino acid sequence of SEQ ID NO:2.

4. The VEGF antagonist of any one of embodiments 1 to 3, wherein X₁ in SEQ ID NO: 2 is valine and X₂ in SEQ ID NO:2 is alanine.

5. The VEGF antagonist of any one of embodiments 1 to 3, wherein X₁ in SEQ ID NO: 2 is isoleucine and X₂ in SEQ ID NO:2 is glycine.

6. The VEGF antagonist of any one of embodiments 1 to 3, wherein the IgM tailpiece comprises the amino acid sequence of SEQ ID NO:3.

7. The VEGF antagonist of any one of embodiments 1 to 3, wherein the IgM tailpiece comprises the amino acid sequence of SEQ ID NO:4.

8. The VEGF antagonist of any one of embodiments 1 to 7, wherein the IgG Fc domain is an IgG1 Fc domain.

9. The VEGF antagonist of any one of embodiments 1 to 7, wherein the IgG Fc domain is an IgG4 Fc domain.

10 The VEGF antagonist of any one of embodiments 1 to 9, wherein the IgG Fc domain has at least 95% sequence identity to the amino acid sequence of SEQ ID NO:1.

11. The VEGF antagonist of any one of embodiments 1 to 9, wherein the IgG Fc domain has at least 96% sequence identity to the amino acid sequence of SEQ ID NO:1.

12. The VEGF antagonist of any one of embodiments 1 to 9, wherein the IgG Fc domain has at least 97% sequence identity to the amino acid sequence of SEQ ID NO: 1.

13. The VEGF antagonist of any one of embodiments 1 to 9, wherein the IgG Fc domain has at least 98% sequence identity to the amino acid sequence of SEQ ID NO:1.

14. The VEGF antagonist of any one of embodiments 1 to 9, wherein the IgG Fc domain has at least 90% sequence identity to amino acids 6 to 232 of the amino acid sequence of SEQ ID NO: 1.

15. The VEGF antagonist of any one of embodiments 1 to 9, wherein the IgG Fc domain has at least 95% sequence identity to amino acids 6 to 232 of the amino acid sequence of SEQ ID NO:1.

16 The VEGF antagonist of any one of embodiments 1 to 9, wherein the IgG Fc domain has at least 96% sequence identity to amino acids 6 to 232 of the amino acid sequence of SEQ ID NO: 1.

17. The VEGF antagonist of any one of embodiments 1 to 13, wherein the IgG Fc domain has at least 97% sequence identity to amino acids 6 to 232 of the amino acid sequence of SEQ ID NO: 1.

18 The VEGF antagonist of any one of embodiments 1 to 9, wherein the IgG Fc domain has at least 98% sequence identity to amino acids 6 to 232 of the amino acid sequence of SEQ ID NO: 1.

19 The VEGF antagonist of any one of embodiments 1 to 9, wherein the IgG Fc domain has at least 90% sequence identity to an amino acid sequence set forth in Table F-1.

20. The VEGF antagonist of any one of embodiments 1 to 9, wherein the IgG Fc domain has at least 95% sequence identity to an amino acid sequence set forth in Table F-1.

21. The VEGF antagonist of any one of embodiments 1 to 9, wherein the IgG Fc domain has at least 98% sequence identity to an amino acid sequence set forth in Table F-1.

22. The VEGF antagonist of any one of embodiments 1 to 21, wherein the IgG Fc domain comprises an amino acid other than cysteine at position 309, optionally the amino acid leucine at position 309, as defined by EU numbering.

23. The VEGF antagonist of any one of embodiments 1 to 21, wherein the IgG Fc domain comprises the amino acid cysteine at position 309, as defined by EU numbering.

24. The VEGF antagonist of any one of embodiments 1 to 23, wherein the IgG Fc domain comprises an amino acid other than cysteine at position 253, optionally the amino acid isoleucine at position 253, as defined by EU numbering.

25. The VEGF antagonist of any one of embodiments 1 to 23, wherein the IgG Fc domain comprises the amino acid cysteine at position 253, as defined by EU numbering.

26. The VEGF antagonist of any one of embodiments 1 to 25, wherein the IgG Fc domain comprises the amino acids aspartic acid at position 359 and leucine at position 361, as defined by EU numbering.

27 The VEGF antagonist of any one of embodiments 1 to 25, wherein the IgG Fc domain comprises the amino acids glutamic acid at position 359 and methionine at position 361, as defined by EU numbering.

28. The VEGF antagonist of any one of embodiments 1 to 27, wherein the IgG Fc domain comprises the amino acids serine at position 442, leucine at position 443, and proline at position 445, as defined by EU numbering.

29. The VEGF antagonist of any one of embodiments 1 to 27, wherein the IgG Fc domain comprises the amino acids aspartic acid at position 442, lysine at position 443, and threonine at position 445, as defined by EU numbering.

30. The VEGF antagonist of any one of embodiments 1 to 29, wherein the IgG Fc domain comprises one or more mutations that decrease affinity for FcRn as compared to an IgG Fc domain having the amino acid sequence of SEQ ID NO:1.

31 The VEGF antagonist of any one of embodiments 1 to 30, wherein the IgG Fc domain comprises amino acid substitutions at one, two, or all three of positions 253, 310, and 435, as defined by EU numbering.

32. The VEGF antagonist of any one of embodiments 1 to 31, wherein the IgG Fc domain comprises the amino acid alanine at position 253, as defined be EU numbering.

33. The VEGF antagonist of any one of embodiments 1 to 32, wherein the IgG Fc domain comprises the amino acid alanine at position 310, as defined be EU numbering.

34. The VEGF antagonist of any one of embodiments 1 to 33, wherein the IgG Fc domain comprises the amino acid alanine, lysine, or arginine at position 435, as defined be EU numbering.

35. The VEGF antagonist of any one of embodiments 1 to 30, wherein the IgG Fc domain comprises amino acid substitutions at positions 253, 310, and 435, as defined by EU numbering.

36. The VEGF antagonist of embodiment 31, wherein the IgG Fc domain comprises the amino acids alanine at position 253, alanine at position 310, and alanine, lysine, or arginine at position 435, as defined by EU numbering.

37 The VEGF antagonist of embodiment 36, wherein the IgG Fc domain comprises the amino acid alanine at position 435 as compared to SEQ ID NO:1, as defined by EU numbering.

38 The VEGF antagonist of embodiment 36, wherein the IgG Fc domain comprises the amino acid lysine at position 435, as defined by EU numbering.

39. The VEGF antagonist of embodiment 36, wherein the IgG Fc domain comprises the amino acid arginine at position 435, as defined by EU numbering.

40. The VEGF antagonist of any one of embodiments 1 to 39, wherein the IgG Fc domain comprises one or more mutations that decrease affinity for a FcγR as compared to an IgG Fc domain having the amino acid sequence of SEQ ID NO:1, e.g., one or more mutations as described in Section 5.4.1.3.

41. The VEGF antagonist of embodiment 40, wherein the FcγR is human FcγRIIIa.

42. The VEGF antagonist of embodiment 40 or 41, wherein the IgG Fc domain comprises amino acid mutations at positions 234, 235, and 329, as defined by EU numbering.

43. The VEGF antagonist of embodiment 40 or 41, wherein the IgG Fc domain comprises the amino acid alanine at position 234, the amino acid alanine at position 235, and the amino acid glycine at position 329, as defined by EU numbering.

44. The VEGF antagonist of embodiment 40, wherein the IgG Fc domain is a chimeric IgG Fc domain having the sequence P-V-A-absent at amino acids 233 to 236, as defined by EU numbering.

45. The VEGF antagonist of embodiment 44, wherein the chimeric IgG Fc domain has an amino acid substitution at position 329, as defined by EU numbering.

46. The VEGF antagonist of embodiment 45, wherein the chimeric IgG Fc domain has the amino acid substitution P329A, as defined by EU numbering.

47. The VEGF antagonist of any one of embodiments 1 to 7, wherein the IgG Fc domain comprises the amino acid sequence of SEQ ID NO:1.

48 The VEGF antagonist of any one of embodiments 1 to 7, wherein the IgG Fc domain comprises amino acids 6 to 232 of the amino acid sequence of SEQ ID NO:1.

49. The VEGF antagonist of any one of embodiments 1 to 7, wherein the IgG Fc domain comprises an amino acid sequence set forth in Table F-1.

50. A VEGF antagonist, optionally the VEGF antagonist of any one of embodiments 1 to 49, comprising five or more dimers, each dimer comprising two polypeptides, each polypeptide comprising, in N- to C-terminal orientation:
(a) a VEGF binding domain, which is optionally (I) a VEGF binding portion of a VEGF antibody or (II) a VEGF binding portion of a VEGF receptor, the VEGF binding portion of the VEGF receptor comprising:
   (i) an Ig-like domain 2 of VEGFR1 comprising an amino acid sequence having at least 90% sequence identity (e.g., at least 91%, at least 92%, at least 93%, at least 94%, or at least 95% sequence identity) to SEQ ID NO: 13; and
   (ii) an Ig-like domain 3 of VEGFR2 comprising an amino acid sequence having at least 90% sequence identity (e.g., at least 91%, at least 92%, at least 93%, at least 94%, or at least 95% sequence identity) to SEQ ID NO: 15 or SEQ ID NO:50; and
(b) a multimerization domain having at least 90% identity to the amino acid sequence of SEQ ID NO:5.

51 The VEGF antagonist of embodiment 50, wherein the multimerization domain has at least 93% identity to the amino acid sequence of SEQ ID NO:5.

52 The VEGF antagonist of embodiment 50, wherein the multimerization domain has at least 95% identity to the amino acid sequence of SEQ ID NO:5.

53. The VEGF antagonist of embodiment 50, wherein the multimerization domain has at least 98% identity to the amino acid sequence of SEQ ID NO:5.

54. The VEGF antagonist of embodiment 50, wherein the multimerization domain has at least 99% identity to the amino acid sequence of SEQ ID NO:5.

55 The VEGF antagonist of embodiment 50, wherein the multimerization domain comprises the amino acid sequence of SEQ ID NO:5.

56. A VEGF antagonist, optionally the VEGF antagonist of any one of embodiments 1 to 49, comprising five or more dimers, each dimer comprising two polypeptides, each polypeptide comprising, in N- to C-terminal orientation:
(a) a VEGF binding domain, which is optionally (I) a VEGF binding portion of a VEGF antibody or (II) a VEGF binding portion of a VEGF receptor, the VEGF binding portion of the VEGF receptor comprising:
   (i) an Ig-like domain 2 of VEGFR1 comprising an amino acid sequence having at least 90% sequence identity (e.g., at least 91%, at least 92%, at least 93%, at least 94%, or at least 95% sequence identity) to SEQ ID NO:13; and
   (ii) an Ig-like domain 3 of VEGFR2 comprising an amino acid sequence having at least 90% sequence identity (e.g., at least 91%, at least 92%, at least 93%, at least 94%, or at least 95% sequence identity) to SEQ ID NO: 15 or SEQ ID NO:50; and
(b) a multimerization domain having at least 90% identity to the amino acid sequence of SEQ ID NO:6.

57 The VEGF antagonist of embodiment 56, wherein the multimerization domain has at least 93% identity to the amino acid sequence of SEQ ID NO:6.

58 The VEGF antagonist of embodiment 56, wherein the multimerization domain has at least 95% identity to the amino acid sequence of SEQ ID NO:6.

59 The VEGF antagonist of embodiment 56, wherein the multimerization domain has at least 98% identity to the amino acid sequence of SEQ ID NO:6.

60. The VEGF antagonist of embodiment 56, wherein the multimerization domain has at least 99% identity to the amino acid sequence of SEQ ID NO:6.

61. The VEGF antagonist of embodiment 56, wherein the multimerization domain comprises the amino acid sequence of SEQ ID NO:6.

62. A VEGF antagonist, optionally the VEGF antagonist of any one of embodiments 1 to 49, comprising five or more dimers, each dimer comprising two polypeptides, each polypeptide comprising, in N- to C-terminal orientation:
(a) a VEGF binding domain, which is optionally (I) a VEGF binding portion of a VEGF antibody or (II) a VEGF binding portion of a VEGF receptor, the VEGF binding portion of the VEGF receptor comprising:
   (i) an Ig-like domain 2 of VEGFR1 comprising an amino acid sequence having at least 90% sequence identity (e.g., at least 91%, at least 92%, at least 93%, at least 94%, or at least 95% sequence identity) to SEQ ID NO: 13; and
   (ii) an Ig-like domain 3 of VEGFR2 comprising an amino acid sequence having at least 90% sequence identity (e.g., at least 91%, at least 92%, at least 93%, at least 94%, or at least 95% sequence identity) to SEQ ID NO: 15 or SEQ ID NO:50; and
(b) a multimerization domain having at least 90% identity to the amino acid sequence of SEQ ID NO:7.

63. The VEGF antagonist of embodiment 62, wherein the multimerization domain has at least 93% identity to the amino acid sequence of SEQ ID NO:7.

64. The VEGF antagonist of embodiment 62, wherein the multimerization domain has at least 95% identity to the amino acid sequence of SEQ ID NO:7.

65. The VEGF antagonist of embodiment 62, wherein the multimerization domain has at least 98% identity to the amino acid sequence of SEQ ID NO:7.

66. The VEGF antagonist of embodiment 62, wherein the multimerization domain has at least 99% identity to the amino acid sequence of SEQ ID NO:7.

67. The VEGF antagonist of embodiment 62, wherein the multimerization domain comprises the amino acid sequence of SEQ ID NO:7.

68. A VEGF antagonist, optionally the VEGF antagonist of any one of embodiments 1 to 49, comprising five or more dimers, each dimer comprising two polypeptides, each polypeptide comprising, in N- to C-terminal orientation:
(a) a VEGF binding domain, which is optionally (I) a VEGF binding portion of a VEGF antibody or (II) a VEGF binding portion of a VEGF receptor, the VEGF binding portion of the VEGF receptor comprising:
   (i) an Ig-like domain 2 of VEGFR1 comprising an amino acid sequence having at least 90% sequence identity (e.g., at least 91%, at least 92%, at least 93%, at least 94%, or at least 95% sequence identity) to SEQ ID NO:13; and
   (ii) an Ig-like domain 3 of VEGFR2 comprising an amino acid sequence having at least 90% sequence identity (e.g., at least 91%, at least 92%, at least 93%, at least 94%, or at least 95% sequence identity) to SEQ ID NO: 15 or SEQ ID NO:50; and
(b) a multimerization domain having at least 90% identity to the amino acid sequence of SEQ ID NO:8.

69. The VEGF antagonist of embodiment 68, wherein the multimerization domain has at least 93% identity to the amino acid sequence of SEQ ID NO:8.

70 The VEGF antagonist of embodiment 68, wherein the multimerization domain has at least 95% identity to the amino acid sequence of SEQ ID NO:8.

71 The VEGF antagonist of embodiment 68, wherein the multimerization domain has at least 98% identity to the amino acid sequence of SEQ ID NO:8.

72 The VEGF antagonist of embodiment 68, wherein the multimerization domain has at least 99% identity to the amino acid sequence of SEQ ID NO:8.

73. The VEGF antagonist of embodiment 68, wherein the multimerization domain comprises the amino acid sequence of SEQ ID NO:8.

74 A VEGF antagonist, optionally the VEGF antagonist of any one of embodiments 1 to 49, comprising five or more dimers, each dimer comprising two polypeptides, each polypeptide comprising, in N- to C-terminal orientation:
(a) a VEGF binding domain, which is optionally (I) a VEGF binding portion of a VEGF antibody or (II) a VEGF binding portion of a VEGF receptor, the VEGF binding portion of the VEGF receptor comprising:
   (i) an Ig-like domain 2 of VEGFR1 comprising an amino acid sequence having at least 90% sequence identity (e.g., at least 91%, at least 92%, at least 93%, at least 94%, or at least 95% sequence identity) to SEQ ID NO:13; and
   (ii) an Ig-like domain 3 of VEGFR2 comprising an amino acid sequence having at least 90% sequence identity (e.g., at least 91%, at least 92%, at least 93%, at least 94%, or at least 95% sequence identity) to SEQ ID NO: 15 or SEQ ID NO:50; and
(b) a multimerization domain having at least 90% identity to the amino acid sequence of SEQ ID NO:9.

75 The VEGF antagonist of embodiment 74, wherein the multimerization domain has at least 93% identity to the amino acid sequence of SEQ ID NO:9.

76 The VEGF antagonist of embodiment 74, wherein the multimerization domain has at least 95% identity to the amino acid sequence of SEQ ID NO:9.

77 The VEGF antagonist of embodiment 74, wherein the multimerization domain has at least 98% identity to the amino acid sequence of SEQ ID NO:9.

78. The VEGF antagonist of embodiment 74, wherein the multimerization domain has at least 99% identity to the amino acid sequence of SEQ ID NO:9.

79 The VEGF antagonist of embodiment 74, wherein the multimerization domain comprises the amino acid sequence of SEQ ID NO:9.

80. A VEGF antagonist, optionally the VEGF antagonist of any one of embodiments 1 to 49, comprising five or more dimers, each dimer comprising two polypeptides, each polypeptide comprising, in N- to C-terminal orientation:
(a) a VEGF binding domain, which is optionally (I) a VEGF binding portion of a VEGF antibody or (II) a VEGF binding portion of a VEGF receptor, the VEGF binding portion of the VEGF receptor comprising:
   (i) an Ig-like domain 2 of VEGFR1 comprising an amino acid sequence having at least 90% sequence identity (e.g., at least 91%, at least 92%, at least 93%, at least 94%, or at least 95% sequence identity) to SEQ ID NO: 13; and
   (ii) an Ig-like domain 3 of VEGFR2 comprising an amino acid sequence having at least 90% sequence identity (e.g., at least 91%, at least 92%, at least 93%, at least 94%, or at least 95% sequence identity) to SEQ ID NO:15 or SEQ ID NO:50; and
(b) a multimerization domain having at least 90% identity to the amino acid sequence of SEQ ID NO: 10.

81. The VEGF antagonist of embodiment 80, wherein the multimerization domain has at least 93% identity to the amino acid sequence of SEQ ID NO: 10.

82 The VEGF antagonist of embodiment 80, wherein the multimerization domain has at least 95% identity to the amino acid sequence of SEQ ID NO: 10.

83 The VEGF antagonist of embodiment 80, wherein the multimerization domain has at least 98% identity to the amino acid sequence of SEQ ID NO: 10.

84 The VEGF antagonist of embodiment 80, wherein the multimerization domain has at least 99% identity to the amino acid sequence of SEQ ID NO: 10.

85. The VEGF antagonist of embodiment 80, wherein the multimerization domain comprises the amino acid sequence of SEQ ID NO: 10.

86. A VEGF antagonist, optionally the VEGF antagonist of any one of embodiments 1 to 49, comprising five or more dimers, each dimer comprising two polypeptides, each polypeptide comprising, in N- to C-terminal orientation:
(a) a VEGF binding domain, which is optionally (I) a VEGF binding portion of a VEGF antibody or (II) a VEGF binding portion of a VEGF receptor, the VEGF binding portion of the VEGF receptor comprising:
   (i) an Ig-like domain 2 of VEGFR1 comprising an amino acid sequence having at least 90% sequence identity (e.g., at least 91%, at least 92%, at least 93%, at least 94%, or at least 95% sequence identity) to SEQ ID NO:13; and
   (ii) an Ig-like domain 3 of VEGFR2 comprising an amino acid sequence having at least 90% sequence identity (e.g., at least 91%, at least 92%, at least 93%, at least 94%, or at least 95% sequence identity) to SEQ ID NO:15 or SEQ ID NO:50; and
(b) a multimerization domain having at least 90% identity to the amino acid sequence of SEQ ID NO: 11.

87 The VEGF antagonist of embodiment 86, wherein the multimerization domain has at least 90% identity to the amino acid sequence of SEQ ID NO: 11.

88. The VEGF antagonist of embodiment 86, wherein the multimerization domain has at least 95% identity to the amino acid sequence of SEQ ID NO: 11.

89. The VEGF antagonist of embodiment 86, wherein the multimerization domain has at least 98% identity to the amino acid sequence of SEQ ID NO: 11.

90 The VEGF antagonist of embodiment 86, wherein the multimerization domain has at least 99% identity to the amino acid sequence of SEQ ID NO: 11.

91. The VEGF antagonist of embodiment 86, wherein the multimerization domain comprises the amino acid sequence of SEQ ID NO: 11.

92. The VEGF antagonist of any one of embodiments 1 to 91, wherein the Ig-like domain 2 of VEGFR1 has at least 96% sequence identity to SEQ ID NO: 13.

93. The VEGF antagonist of any one of embodiments 1 to 91, wherein the Ig-like domain 2 of VEGFR1 has at least 97% sequence identity to SEQ ID NO: 13.

94 The VEGF antagonist of any one of embodiments 1 to 91, wherein the Ig-like domain 2 of VEGFR1 has at least 98% sequence identity to SEQ ID NO: 13.

95. The VEGF antagonist of any one of embodiments 1 to 91, wherein the Ig-like domain 2 of VEGFR1 has at least 99% sequence identity to SEQ ID NO: 13.

96. The VEGF antagonist of any one of embodiments 1 to 91, wherein the Ig-like domain 2 of VEGFR1 comprises the amino acid sequence of SEQ ID NO: 13.

97. The VEGF antagonist of any one of embodiments 1 to 96, wherein the Ig-like domain 2 of VEGFR1 has at least 95% sequence identity to SEQ ID NO: 14.

98 The VEGF antagonist of any one of embodiments 1 to 96, wherein the Ig-like domain 2 of VEGFR1 has at least 96% sequence identity to SEQ ID NO: 14.

99. The VEGF antagonist of any one of embodiments 1 to 96, wherein the Ig-like domain 2 of VEGFR1 has at least 97% sequence identity to SEQ ID NO: 14.

100. The VEGF antagonist of any one of embodiments 1 to 96, wherein the Ig-like domain 2 of VEGFR1 has at least 98% sequence identity to SEQ ID NO: 14.

101. The VEGF antagonist of any one of embodiments 1 to 96, wherein the Ig-like domain 2 of VEGFR1 has at least 99% sequence identity to SEQ ID NO: 14.

102. The VEGF antagonist of any one of embodiments 1 to 96, wherein the Ig-like domain 2 of VEGFR1 comprises the amino acid sequence of SEQ ID NO: 14.

103. The VEGF antagonist of any one of embodiments 1 to 102, wherein the Ig-like domain 3 of VEGFR2 has at least 96% sequence identity to SEQ ID NO: 15 or SEQ ID NO:50.

104. The VEGF antagonist of any one of embodiments 1 to 102, wherein the Ig-like domain 3 of VEGFR2 has at least 97% sequence identity to SEQ ID NO: 15 or SEQ ID NO:50.

105. The VEGF antagonist of any one of embodiments 1 to 102, wherein the Ig-like domain 3 of VEGFR2 has at least 98% sequence identity to SEQ ID NO: 15 or SEQ ID NO:50.

106. The VEGF antagonist of any one of embodiments 1 to 102, wherein the Ig-like domain 3 of VEGFR2 has at least 99% sequence identity to SEQ ID NO: 15 or SEQ ID NO:50.

107. The VEGF antagonist of any one of embodiments 1 to 106, wherein the Ig-like domain 3 of VEGFR2 comprises SEQ ID NO: 15 or SEQ ID NO:50.

108. The VEGF antagonist of any one of embodiments 1 to 107, wherein the VEGF binding domain comprises an amino acid sequence having at least 90% identity to SEQ ID NO: 16.

109. The VEGF antagonist of any one of embodiments 1 to 107, wherein the VEGF binding domain comprises an amino acid sequence having at least 93% identity to SEQ ID NO: 16.

110. The VEGF antagonist of any one of embodiments 1 to 107, wherein the VEGF binding domain comprises an amino acid sequence having at least 94% identity to SEQ ID NO: 16.

111. The VEGF antagonist of any one of embodiments 1 to 107, wherein the VEGF binding domain comprises an amino acid sequence having at least 95% identity to SEQ ID NO: 16.

112. The VEGF antagonist of any one of embodiments 1 to 107, wherein the VEGF binding domain comprises an amino acid sequence having at least 96% identity to SEQ ID NO: 16.

113. The VEGF antagonist of any one of embodiments 1 to 107, wherein the VEGF binding domain comprises an amino acid sequence having at least 97% identity to SEQ ID NO: 16.

114. The VEGF antagonist of any one of embodiments 1 to 107, wherein the VEGF binding domain comprises an amino acid sequence having at least 98% identity to SEQ ID NO: 16.

115. The VEGF antagonist of any one of embodiments 1 to 107, wherein the VEGF binding domain comprises an amino acid sequence having at least 99% identity to SEQ ID NO: 16.

116. The VEGF antagonist of any one of embodiments 1 to 107, wherein the VEGF binding domain comprises an amino acid sequence having at least 99.5% identity to SEQ ID NO: 16.

117. The VEGF antagonist of any one of embodiments 1 to 107, wherein the VEGF binding domain comprises the amino acid sequence of SEQ ID NO:16.

118. A VEGF antagonist, optionally the VEGF antagonist of any one of embodiments 1 to 117, comprising five or more dimers, each dimer comprising two polypeptides, each comprising an amino acid sequence having at least 90% sequence identity to the amino acid sequence of SEQ ID NO: 18.

119. The VEGF antagonist of embodiment 118, wherein each polypeptide comprises an amino acid sequence having at least 93% identity to SEQ ID NO: 18.

120. The VEGF antagonist of embodiment 118, wherein each polypeptide comprises an amino acid sequence having at least 95% identity to SEQ ID NO: 18.

121. The VEGF antagonist of embodiment 118, wherein each polypeptide comprises an amino acid sequence having at least 96% identity to SEQ ID NO: 18.

122. The VEGF antagonist of embodiment 118, wherein each polypeptide comprises an amino acid sequence having at least 97% identity to SEQ ID NO: 18.

123. The VEGF antagonist of embodiment 118, wherein each polypeptide comprises an amino acid sequence having at least 98% identity to SEQ ID NO: 18.

124. The VEGF antagonist of embodiment 118, wherein each polypeptide comprises an amino acid sequence having at least 99% identity to SEQ ID NO: 18.

125. The VEGF antagonist of embodiment 118, wherein each polypeptide comprises the amino acid sequence of SEQ ID NO: 18.

126. A VEGF antagonist, optionally the VEGF antagonist of any one of embodiments 1 to 117, comprising five or more dimers, each dimer comprising two polypeptides, each comprising an amino acid sequence having at least 90% sequence identity to the amino acid sequence of SEQ ID NO: 19.

127. The VEGF antagonist of embodiment 126, wherein each polypeptide comprises an amino acid sequence having at least 93% identity to SEQ ID NO: 19.

128. The VEGF antagonist of embodiment 126, wherein each polypeptide comprises an amino acid sequence having at least 95% identity to SEQ ID NO: 19.

129. The VEGF antagonist of embodiment 126, wherein each polypeptide comprises an amino acid sequence having at least 96% identity to SEQ ID NO: 19.

130. The VEGF antagonist of embodiment 126, wherein each polypeptide comprises an amino acid sequence having at least 97% identity to SEQ ID NO: 19.

131. The VEGF antagonist of embodiment 126, wherein each polypeptide comprises an amino acid sequence having at least 98% identity to SEQ ID NO: 19.

132. The VEGF antagonist of embodiment 126, wherein each polypeptide comprises an amino acid sequence having at least 99% identity to SEQ ID NO: 19.

133. The VEGF antagonist of embodiment 126, wherein each polypeptide comprises the amino acid sequence of SEQ ID NO:19.

134. A VEGF antagonist, optionally the VEGF antagonist of any one of embodiments 1 to 117, comprising five or more dimers, each dimer comprising two polypeptides, each comprising an amino acid sequence having at least 90% sequence identity to the amino acid sequence of SEQ ID NO:20.

135. The VEGF antagonist of embodiment 134, wherein each polypeptide comprises an amino acid sequence having at least 93% identity to SEQ ID NO:20.

136. The VEGF antagonist of embodiment 134, wherein each polypeptide comprises an amino acid sequence having at least 95% identity to SEQ ID NO:20.

137. The VEGF antagonist of embodiment 134, wherein each polypeptide comprises an amino acid sequence having at least 96% identity to SEQ ID NO:20.

138. The VEGF antagonist of embodiment 134, wherein each polypeptide comprises an amino acid sequence having at least 97% identity to SEQ ID NO:20.

139. The VEGF antagonist of embodiment 134, wherein each polypeptide comprises an amino acid sequence having at least 98% identity to SEQ ID NO:20.

140. The VEGF antagonist of embodiment 134, wherein each polypeptide comprises an amino acid sequence having at least 99% identity to SEQ ID NO:20.

141. The VEGF antagonist of embodiment 134, wherein each polypeptide comprises the amino acid sequence of SEQ ID NO:20.

142. A VEGF antagonist, optionally the VEGF antagonist of any one of embodiments 1 to 117, comprising five or more dimers, each dimer comprising two polypeptides, each comprising an amino acid sequence having at least 90% sequence identity to the amino acid sequence of SEQ ID NO:21.

143. The VEGF antagonist of embodiment 142, wherein each polypeptide comprises an amino acid sequence having at least 93% identity to SEQ ID NO:21.

144. The VEGF antagonist of embodiment 142, wherein each polypeptide comprises an amino acid sequence having at least 95% identity to SEQ ID NO:21.

145. The VEGF antagonist of embodiment 142, wherein each polypeptide comprises an amino acid sequence having at least 96% identity to SEQ ID NO:21.

146. The VEGF antagonist of embodiment 142, wherein each polypeptide comprises an amino acid sequence having at least 97% identity to SEQ ID NO:21.

147. The VEGF antagonist of embodiment 142, wherein each polypeptide comprises an amino acid sequence having at least 98% identity to SEQ ID NO:21.

148. The VEGF antagonist of embodiment 142, wherein each polypeptide comprises an amino acid sequence having at least 99% identity to SEQ ID NO:21.

149. The VEGF antagonist of embodiment 142, wherein each polypeptide comprises the amino acid sequence of SEQ ID NO:21.

150. The VEGF antagonist of any one of embodiments 1 to 149, wherein the VEGF antagonist comprises six dimers.

151. A VEGF antagonist comprising:
(a) a VEGF binding domain; and
(b) a multimerization domain comprising:
   (i) a chimeric IgG Fc domain comprising an amino acid sequence that has at least 90% identity to SEQ ID NO:1, provided that the chimeric IgG Fc domain has the sequence P-V-A-absent at amino acids 233 to 236 and the amino acid substitution P329A as compared to SEQ ID NO:1, as defined by EU numbering; and
   (ii) optionally, an IgM tailpiece having at least 80% sequence identity to the amino acid sequence of SEQ ID NO:2.

152. The VEGF antagonist of embodiment 151, wherein the VEGF binding domain is a Fab or component thereof.

153. The VEGF antagonist of embodiment 151, wherein the VEGF binding domain is an scFv.

154. The VEGF antagonist of any one of embodiments 151 to 153, wherein the VEGF binding domain specifically binds to human VEGF.

155. The VEGF antagonist of any one of embodiments 151 to 154, wherein the VEGF binding domain comprises heavy chain and light chain CDRs of an antibody set forth in Table T1.

156. The VEGF antagonist of any one of embodiments 151 to 155, wherein the first target binding domain comprises a VH and VL of an antibody set forth in Table T1.

157. The VEGF antagonist of any one of embodiments 151 to 154, wherein the VEGF binding domain comprises a VEGF receptor or portion thereof.

158. The VEGF antagonist of embodiment 157, wherein the VEGF binding domain comprises, in N- to C-terminal orientation, an Ig-like domain 2 of VEGFR1 and an Ig-like domain 3 of VEGFR2.

159. The VEGF antagonist of embodiment 158, wherein the Ig-like domain 2 of VEGFR1 has at least 96% sequence identity to SEQ ID NO: 13.

160. The VEGF antagonist of embodiment 158, wherein the Ig-like domain 2 of VEGFR1 has at least 97% sequence identity to SEQ ID NO: 13.

161. The VEGF antagonist of embodiment 158, wherein the Ig-like domain 2 of VEGFR1 has at least 98% sequence identity to SEQ ID NO: 13.

162. The VEGF antagonist of embodiment 158, wherein the Ig-like domain 2 of VEGFR1 has at least 99% sequence identity to SEQ ID NO: 13.

163. The VEGF antagonist of embodiment 158, wherein the Ig-like domain 2 of VEGFR1 comprises the amino acid sequence of SEQ ID NO: 13.

164. The VEGF antagonist of any one of embodiments 158 to 163, wherein the Ig-like domain 2 of VEGFR1 comprises an amino acid sequence having at least 95% sequence identity to SEQ ID NO: 14.

165. The VEGF antagonist of any one of embodiments 158 to 163, wherein the Ig-like domain 2 of VEGFR1 comprises an amino acid sequence having at least 96% sequence identity to SEQ ID NO: 14.

166. The VEGF antagonist of any one of embodiments 158 to 163, wherein the Ig-like domain 2 of VEGFR1 comprises an amino acid sequence having at least 97% sequence identity to SEQ ID NO: 14.

167. The VEGF antagonist of any one of embodiments 158 to 163, wherein the Ig-like domain 2 of VEGFR1 comprises an amino acid sequence having at least 98% sequence identity to SEQ ID NO: 14.

168. The VEGF antagonist of any one of embodiments 158 to 163, wherein the Ig-like domain 2 of VEGFR1 comprises an amino acid sequence having at least 99% sequence identity to SEQ ID NO: 14.

169. The VEGF antagonist of any one of embodiments 158 to 163, wherein the Ig-like domain 2 of VEGFR1 comprises the amino acid sequence of SEQ ID NO: 14.

170. The VEGF antagonist of any one of embodiments 158 to 169, wherein the Ig-like domain 3 of VEGFR2 has at least 96% sequence identity to SEQ ID NO: 15 or SEQ ID NO:50.

171. The VEGF antagonist of any one of embodiments 158 to 169, wherein the Ig-like domain 3 of VEGFR2 has at least 97% sequence identity to SEQ ID NO: 15 or SEQ ID NO:50.

172. The VEGF antagonist of any one of embodiments 158 to 169, wherein the Ig-like domain 3 of VEGFR2 has at least 98% sequence identity to SEQ ID NO: 15 or SEQ ID NO:50.

173. The VEGF antagonist of any one of embodiments 158 to 169, wherein the Ig-like domain 3 of VEGFR2 has at least 99% sequence identity to SEQ ID NO: 15 or SEQ ID NO:50.

174. The VEGF antagonist of any one of embodiments 158 to 169, wherein the Ig-like domain 3 of VEGFR2 comprises SEQ ID NO: 15 or SEQ ID NO:50.

175. The VEGF antagonist of any one of embodiments 158 to 174, wherein the VEGF binding domain comprises an amino acid sequence having at least 90% identity to SEQ ID NO: 16.

176. The VEGF antagonist of any one of embodiments 158 to 174, wherein the VEGF binding domain comprises an amino acid sequence having at least 93% identity to SEQ ID NO: 16.

177. The VEGF antagonist of any one of embodiments 158 to 174, wherein the VEGF binding domain comprises an amino acid sequence having at least 94% identity to SEQ ID NO: 16.

178. The VEGF antagonist of any one of embodiments 158 to 174, wherein the VEGF binding domain comprises an amino acid sequence having at least 95% identity to SEQ ID NO: 16.

179. The VEGF antagonist of any one of embodiments 158 to 174, wherein the VEGF binding domain comprises an amino acid sequence having at least 96% identity to SEQ ID NO: 16.

180. The VEGF antagonist of any one of embodiments 158 to 174, wherein the VEGF binding domain comprises an amino acid sequence having at least 97% identity to SEQ ID NO: 16.

181. The VEGF antagonist of any one of embodiments 158 to 174, wherein the VEGF binding domain comprises an amino acid sequence having at least 98% identity to SEQ ID NO: 16.

182. The VEGF antagonist of any one of embodiments 158 to 174, wherein the VEGF binding domain comprises an amino acid sequence having at least 99% identity to SEQ ID NO: 16.

183. The VEGF antagonist of any one of embodiments 158 to 174, wherein the VEGF binding domain comprises the amino acid sequence of SEQ ID NO: 16.

184. The VEGF antagonist of any one of embodiments 158 to 182, wherein the VEGF antagonist comprises the IgM tailpiece.

185. The VEGF antagonist of any one of embodiments 158 to 184, wherein the IgM tailpiece has at least 85% sequence identity to the amino acid sequence of SEQ ID NO:2.

186. The VEGF antagonist of any one of embodiments 158 to 184, wherein the IgM tailpiece has at least 90% sequence identity to the amino acid sequence of SEQ ID NO:2.

187. The VEGF antagonist of any one of embodiments 158 to 186, wherein X₁ in SEQ ID NO: 2 is valine and X₂ in SEQ ID NO:2 is alanine.

188. The VEGF antagonist of any one of embodiments 158 to 186, wherein X₁ in SEQ ID NO: 2 is isoleucine and X₂ in SEQ ID NO:2 is glycine.

189. The VEGF antagonist of any one of embodiments 158 to 186, wherein the IgM tailpiece comprises the amino acid sequence of SEQ ID NO:3.

190. The VEGF antagonist of any one of embodiments 158 to 186, wherein the IgM tailpiece comprises the amino acid sequence of SEQ ID NO:4.

191. The VEGF antagonist of any one of embodiments 151 to 153, wherein the VEGF binding domain comprises means for binding human VEGF.

192. The VEGF antagonist of any one of embodiments 158 to 191, wherein the VEGF antagonist comprises a dimer comprising two polypeptides, each polypeptide comprising the VEGF binding domain, the chimeric IgG Fc domain, and, optionally, the IgM tailpiece.

193. The VEGF antagonist of embodiment 192, wherein the VEGF antagonist comprises five or more dimers.

194. The VEGF antagonist of embodiment 192 or 193, wherein the VEGF antagonist comprises six dimers.

195. A composition comprising a population of VEGF antagonists according to any one of embodiments 1 to 194.

196. The composition of embodiment 195, wherein at least 80% of the population of VEGF antagonists have a molecular weight within a 5% range.

197. The composition of embodiment 195 or embodiment 196, wherein at least 80% of the population of VEGF antagonists have a molecular weight of greater than 500 kDa.

198. The composition of any one of embodiments 195 to 197, wherein at least 80% of the population of VEGF antagonists are molecules comprising five or more dimers.

199. The composition of any one of embodiments 195 to 198, wherein at least 80% of the population of VEGF antagonists are molecules comprising six dimers.

200. The composition of any one of embodiments 195 to 199, wherein the population of VEGF antagonists is at least 80% homogeneous.

201. The composition of any one of embodiments 195 to 200, wherein at least 80% of the population of VEGF antagonists elude within a 5% range as measured by size-exclusion ultra performance liquid chromatography (SE-UPLC) analysis.

202. The composition of any one of embodiments 195 to 201, wherein the composition retains one or more of the properties recited in any one of embodiments 151 to 201 after storage at 4° C. for 28 days.

203. The composition of any one of embodiments 195 to 202, which is a pharmaceutical composition comprising one or more pharmaceutically acceptable carriers or excipients.

204. A nucleic acid or plurality of nucleic acids encoding the polypeptides of the VEGF antagonist of any one of embodiments 1 to 194.

205. A host cell engineered to express the nucleic acid(s) of embodiment 204.

206. A cell transfected with one or more expression vectors comprising one or more nucleic acid sequences encoding the polypeptides of the VEGF antagonist of any one of embodiments 1 to 194.

207. A method of producing the VEGF antagonist of any one of embodiments 1 to 194 comprising culturing the cell of embodiment 205 or 206 under conditions sufficient to express the polypeptides.

208. A method of reducing systemic exposure of a VEGF antagonist comprising administering to a subject the VEGF antagonist of any one of embodiments 1 to 194 or the composition of any one of embodiments 195 to 203.

209. A method of inhibiting VEGF receptor activity in a cell comprising contacting the cell with the VEGF antagonist of any one of embodiments 1 to 194 or the composition of any one of embodiments 195 to 203.

210. A method of inhibiting VEGF receptor activity in a subject comprising administering to a subject the VEGF antagonist of any one of embodiments 1 to 194 or the composition of any one of embodiments 195 to 203.

211. A method for treating an angiogenic eye disorder in a subject comprising administering to the subject the VEGF antagonist of any one of embodiments 1 to 194 or the composition of any one of embodiments 195 to 203.

212. The method of embodiment 211, wherein the angiogenic eye disorder is age related macular degeneration, diabetic retinopathy, diabetic macular edema, central retinal vein occlusion, branch retinal vein occlusion, or corneal neovascularization.

213. The method of embodiment 211, wherein the angiogenic eye disorder is age related macular degeneration.

214. The method of embodiment 211, wherein the angiogenic eye disorder is diabetic retinopathy.

215. The method of embodiment 211, wherein the angiogenic eye disorder is diabetic macular edema.

216. The method of embodiment 211, wherein the angiogenic eye disorder is central retinal vein occlusion.

217. The method of embodiment 211, wherein the angiogenic eye disorder is branch retinal vein occlusion.

218. The method of embodiment 211, wherein the angiogenic eye disorder is corneal neovascularization.

219. The method of embodiment 211, wherein the VEGF antagonist is administered to the subject intravitreally.

### 8. EXAMPLES

### 8.1. Materials and Methods for Examples 1 to 4

### 8.1.1. Design of Hexameric VEGF Antagonists

Various multimerization domains were engineered, each comprising, in N- to C-terminal order, a hinge domain (in some cases a truncated IgG1 hinge domain lacking the first five amino acids, EPKSC (SEQ ID NO: 80)), an IgG1 CH2 domain, an IgG1 CH3 domain, and an IgM tailpiece (as depicted in FIG. 1A). Sequences of the engineered multimerization domains are provided in Table E1, below.

TABLE E1
Engineered Multimerization domain Sequences

| | | SEQ ID |
|---|---|---|
| Name | Sequence | NO: |
| IgG1-T- | DKTHTCPPCPAPELLGGPSVFLFPPKPKDTLMISRTPEVTCVVVDVSHEDPEVK | 5 |
| Hex(WT) | FNWYVDGVEVHNAKTKPREEQYNSTYRVVSVLTVLHQDWLNGKEYKCKVSNKAL | |
| | PAPIEKTISKAKGQPREPQVYTLPPSRDELTKNQVSLTCLVKGFYPSDIAVEWE | |
| | SNGQPENNYKTTPPVLDSDGSFFLYSKLTVDKSRWQQGNVFSCSVMHEALHNHY | |
| | TQKSLDKSTGKPTLYNVSLVMSDTAGTCY | |
| IgG1-T- | EPKSCDKTHTCPPCPAPELLGGPSVFLFPPKPKDTLMISRTPEVTCVVVDVSHE | 6 |
| Hex(Mut) | DPEVKFNWYVDGVEVHNAKTKPREEQYNSTYRVVSVLTVCHQDWLNGKEYKCKV | |
| | SNKALPAPIEKTISKAKGQPREPQVYTLPPSREEMTKNQVSLTCLVKGFYPSDI | |
| | AVEWESNGQPENNYKTTPPVLDSDGSFFLYSKLTVDKSRWQQGNVFSCSVMHEA | |
| | LHNHYTQKSLDKSTGKPTLYNVSLIMSDTG | |
| IgG1-T- | DKTHTCPPCPAPELLGGPSVFLFPPKPKDTLMISRTPEVTCVVVDVSHEDPEVK | 7 |
| Hex(Mut1) | FNWYVDGVEVHNAKTKPREEQYNSTYRVVSVLTVCHQDWLNGKEYKCKVSNKAL | |
| | PAPIEKTISKAKGQPREPQVYTLPPSREEMTKNQVSLTCLVKGFYPSDIAVEWE | |
| | SNGQPENNYKTTPPVLDSDGSFFLYSKLTVDKSRWQQGNVFSCSVMHEALHNHY | |
| | TQKSLDKSTGKPTLYNVSLIMSDTGGTCY | |
| IgG1-T- | DKTHTCPPCPAPELLGGPSVFLFPPKPKDTLMISRTPEVTCVVVDVSHEDPEVK | 33 |
| Hex | FNWYVDGVEVHNAKTKPREEQYNSTYRVVSVLTVCHQDWLNGKEYKCKVSNKAL | |
| (Mut1-V2) | PAPIEKTISKAKGQPREPQVYTLPPSREEMTKNQVSLTCLVKGFYPSDIAVEWE | |
| | SNGQPENNYKTTPPVLDSDGSFFLYSKLTVDKSRWQQGNVFSCSVMHEALHNHY | |
| | TQKSLDKSTGKPTLYNVSLVMSDTAGTCY | |
| IgG1-T- | DKTHTCPPCPAPELLGGPSVFLFPPKPKDTLMCSRTPEVTCVVVDVSHEAPEVK | 8 |
| Hex | FNWYVDGVEVHNAKTKPREEQYNSTYRVVSVLTVLHQDWLNGKEYKCKVSNKAL | |
| (Mut2) | PAGIEKTISKAKGQPREPQVYTLPPSRDELTKNQVSLTCLVKGFYPSDIAVEWE | |
| | SNGQPENNYKTTPPVLDSDGSFFLYSKLTVDKSRWQQGNVFSCSVMHEALHNHY | |
| | TQKSLSLSPGKPTLYNVSLVMSDTAGTCY | |
| IgG1-T- | DKTHTCPPCPAPEAAGGPSVFLFPPKPKDTLMISRTPEVTCVVVDVSHEDPEVK | 9 |
| Mut1-V2 | FNWYVDGVEVHNAKTKPREEQYNSTYRVVSVLTVCHQDWLNGKEYKCKVSNKAL | |
| LALAPG | GAPIEKTISKAKGQPREPQVYTLPPSREEMTKNQVSLTCLVKGFYPSDIAVEWE | |
| | SNGQPENNYKTTPPVLDSDGSFFLYSKLTVDKSRWQQGNVFSCSVMHEALHNHY | |
| | TQKSLDKSTGKPTLYNVSLVMSDTAGTCY | |
| IgG1-T- | DKTHTCPPCPAPPVAGPSVFLFPPKPKDTLMISRTPEVTCVVVDVSHEDPEVKF | 10 |
| Mut1-V2 | NWYVDGVEVHNAKTKPREEQYNSTYRVVSVLTVCHQDWLNGKEYKCKVSNKALA | |
| PVA/ | APIEKTISKAKGQPREPQVYTLPPSREEMTKNQVSLTCLVKGFYPSDIAVEWES | |
| P329A | NGQPENNYKTTPPVLDSDGSFFLYSKLTVDKSRWQQGNVFSCSVMHEALHNHYT | |
| | QKSLDKSTGKPTLYNVSLVMSDTAGTCY | |
| IgG1-T- | DKTHTCPPCPAPEAAGGPSVFLFPPKPKDTLMCSRTPEVTCVVVDVSHEAPEVK | 11 |
| Mut2 | FNWYVDGVEVHNAKTKPREEQYNSTYRVVSVLTVLHQDWLNGKEYKCKVSNKAL | |
| LALAPG | GAGIEKTISKAKGQPREPQVYTLPPSRDELTKNQVSLTCLVKGFYPSDIAVEWE | |
| | SNGQPENNYKTTPPVLDSDGSFFLYSKLTVDKSRWQQGNVFSCSVMHEALHNHY | |
| | TQKSLSLSPGKPTLYNVSLVMSDTAGTCY | |
| IgG4/ | ESKYGPPCPPCPAPGGGGPSVFLFPPKPKDTLMISRTPEVTCVVVDVSQEDPEV | 12 |
| IgG1-T- | QFNWYVDGVEVHNAKTKPREEQFNSTYRVVSVLTVCHQDWLNGKEYKCKVSNKG | |
| Mut1 | LPSSIEKTISKAKGQPREPQVYTLPPSREEMTKNQVSLTCLVKGFYPSDIAVEW | |
| | ESNGQPENNYKTTPPVLDSDGSFFLYSKLTVDKSRWQQGNVFSCSVMHEALHNH | |
| | YTQKSLDKSTGKPTLYNVSLVMSDTAGTCY | |

8.1.2. Production of Hexameric IgG-Tail Piece Constructs with VEGF Targeting Domains

Hexameric VEGF antagonists were constructed with a VEGF binding domain comprising an Ig-like domain 2 of VEGFR1 (comprising the amino acid sequence of SEQ ID NO: 14) and an Ig-like domain 3 of VEGFR2 (comprising the amino acid sequence of SEQ ID NO:15), fused directly to the hinge domains (FIGS. 1C and 1D). The sequence of the VEGF binding domain used in the constructs described in the Examples is provided in Table E2, below.

TABLE E2
REGN3 sequence

| | | SEQ ID |
|---|---|---|
| Name | Sequence | NO: |
| VEGF | DTGRPFVEMYSEIPEIIHMTEGRE | 16 |
| binding | LVIPCRVTSPNITVTLKKFPLDTL | |
| domain | IPDGKRIIWDSRKGFIISNATYKE | |
| | IGLLTCEATVNGHLYKTNYLTHRQ | |
| | TNTIIDVVLSPSHGIELSVGEKLV | |
| | LNCTARTELNVGIDFNWEYPSSKH | |
| | QHKKLVNRDLKTQSGSEMKKFLST | |
| | LTIDGVTRSDQGLYTCAASSGLMT | |
| | KKNSTFVRVHEK | |

Sequences of hexameric VEGF antagonists (“Hex-REGN3” constructs) are provided in Table E3 below.

TABLE E3
Hexameric VEGF Antagonist Sequences

| | | SEQ ID |
|---|---|---|
| Name | Sequence | NO: |
| HEX-REGN3 | SDTGRPFVEMYSEIPEIIHMTEGRELVIPCRVTSPNITVTLKKFPLDTLI | 36 |
| (IgG1) WT | PDGKRIIWDSRKGFIISNATYKEIGLLTCEATVNGHLYKTNYLTHRQTNT | |
| LALAPG | IIDVVLSPSHGIELSVGEKLVLNCTARTELNVGIDFNWEYPSSKHQHKKL | |
| | VNRDLKTQSGSEMKKFLSTLTIDGVTRSDQGLYTCAASSGLMTKKNSTFV | |
| | RVHEKDKTHTCPPCPAPEAAGGPSVFLFPPKPKDTLMISRTPEVTCVVVD | |
| | VSHEDPEVKFNWYVDGVEVHNAKTKPREEQYNSTYRVVSVLTVLHQDWLN | |
| | GKEYKCKVSNKALGAPIEKTISKAKGQPREPQVYTLPPSRDELTKNQVSL | |
| | TCLVKGFYPSDIAVEWESNGQPENNYKTTPPVLDSDGSFFLYSKLTVDKS | |
| | RWQQGNVFSCSVMHEALHNHYTQKSLSLSPGKPTLYNVSLVMSDTAGTCY | |
| HEX-REGN3 | SDTGRPFVEMYSEIPEIIHMTEGRELVIPCRVTSPNITVTLKKFPLDTLI | 37 |
| (IgG1) | PDGKRIIWDSRKGFIISNATYKEIGLLTCEATVNGHLYKTNYLTHRQTNT | |
| Mut 1 | IIDVVLSPSHGIELSVGEKLVLNCTARTELNVGIDFNWEYPSSKHQHKKL | |
| LALAPG | VNRDLKTQSGSEMKKFLSTLTIDGVTRSDQGLYTCAASSGLMTKKNSTFV | |
| | RVHEKDKTHTCPPCPAPEAAGGPSVFLFPPKPKDTLMISRTPEVTCVVVD | |
| | VSHEDPEVKFNWYVDGVEVHNAKTKPREEQYNSTYRVVSVLTVCHQDWLN | |
| | GKEYKCKVSNKALGAPIEKTISKAKGQPREPQVYTLPPSREEMTKNQVSL | |
| | TCLVKGFYPSDIAVEWESNGQPENNYKTTPPVLDSDGSFFLYSKLTVDKS | |
| | RWQQGNVFSCSVMHEALHNHYTQKSLDKSTGKPTLYNVSLIMSDTGGTCY | |
| Hex-REGN3 | SDTGRPFVEMYSEIPEIIHMTEGRELVIPCRVTSPNITVTLKKFPLDTLI | 17 |
| (IgG1) | PDGKRIIWDSRKGFIISNATYKEIGLLTCEATVNGHLYKTNYLTHRQTNT | |
| Mut1-V2 | IIDVVLSPSHGIELSVGEKLVLNCTARTELNVGIDFNWEYPSSKHQHKKL | |
| LALAPG | VNRDLKTQSGSEMKKFLSTLTIDGVTRSDQGLYTCAASSGLMTKKNSTFV | |
| | RVHEKDKTHTCPPCPAPEAAGGPSVFLFPPKPKDTLMISRTPEVTCVVVD | |
| | VSHEDPEVKFNWYVDGVEVHNAKTKPREEQYNSTYRVVSVLTVCHQDWLN | |
| | GKEYKCKVSNKALGAPIEKTISKAKGQPREPQVYTLPPSREEMTKNQVSL | |
| | TCLVKGFYPSDIAVEWESNGQPENNYKTTPPVLDSDGSFFLYSKLTVDKS | |
| | RWQQGNVFSCSVMHEALHNHYTQKSLDKSTGKPTLYNVSLVMSDTAGTCY | |
| Hex-REGN3 | SDTGRPFVEMYSEIPEIIHMTEGRELVIPCRVTSPNITVTLKKFPLDTLI | 18 |
| (IgG1) | PDGKRIIWDSRKGFIISNATYKEIGLLTCEATVNGHLYKTNYLTHRQTNT | |
| Mut1-V2 | IIDVVLSPSHGIELSVGEKLVLNCTARTELNVGIDFNWEYPSSKHQHKKL | |
| PVA/P329A | VNRDLKTQSGSEMKKFLSTLTIDGVTRSDQGLYTCAASSGLMTKKNSTFV | |
| | RVHEKDKTHTCPPCPAPPVAGPSVFLFPPKPKDTLMISRTPEVTCVVVDV | |
| | SHEDPEVKFNWYVDGVEVHNAKTKPREEQYNSTYRVVSVLTVCHQDWLNG | |
| | KEYKCKVSNKALAAPIEKTISKAKGQPREPQVYTLPPSREEMTKNQVSLT | |
| | CLVKGFYPSDIAVEWESNGQPENNYKTTPPVLDSDGSFFLYSKLTVDKSR | |
| | WQQGNVFSCSVMHEALHNHYTQKSLDKSTGKPTLYNVSLVMSDTAGTCY | |
| Hex-REGN3 | SDTGRPFVEMYSEIPEIIHMTEGRELVIPCRVTSPNITVTLKKFPLDTLI | 19 |
| (IgG1) | PDGKRIIWDSRKGFIISNATYKEIGLLTCEATVNGHLYKTNYLTHRQTNT | |
| Mut2 | IIDVVLSPSHGIELSVGEKLVLNCTARTELNVGIDFNWEYPSSKHQHKKL | |
| LALAPG | VNRDLKTQSGSEMKKFLSTLTIDGVTRSDQGLYTCAASSGLMTKKNSTFV | |
| | RVHEKDKTHTCPPCPAPEAAGGPSVFLFPPKPKDTLMCSRTPEVTCVVVD | |
| | VSHEAPEVKFNWYVDGVEVHNAKTKPREEQYNSTYRVVSVLTVLHQDWLN | |
| | GKEYKCKVSNKALGAGIEKTISKAKGQPREPQVYTLPPSRDELTKNQVSL | |
| | TCLVKGFYPSDIAVEWESNGQPENNYKTTPPVLDSDGSFFLYSKLTVDKS | |
| | RWQQGNVFSCSVMHEALHNHYTQKSLSLSPGKPTLYNVSLVMSDTAGTCY | |
| Hex-REGN3 | SDTGRPFVEMYSEIPEIIHMTEGRELVIPCRVTSPNITVTLKKFPLDTLI | 20 |
| (IgG4/IgG1 | PDGKRIIWDSRKGFIISNATYKEIGLLTCEATVNGHLYKTNYLTHRQTNT | |
| chimera) | IIDVVLSPSHGIELSVGEKLVLNCTARTELNVGIDFNWEYPSSKHQHKKL | |
| Mut1-V2 | VNRDLKTQSGSEMKKFLSTLTIDGVTRSDQGLYTCAASSGLMTKKNSTFV | |
| | RVHEKESKYGPPCPPCPAPGGGGPSVFLFPPKPKDTLMISRTPEVTCVVV | |
| | DVSQEDPEVQFNWYVDGVEVHNAKTKPREEQFNSTYRVVSVLTVCHQDWL | |
| | NGKEYKCKVSNKGLPSSIEKTISKAKGQPREPQVYTLPPSREEMTKNQVS | |
| | LTCLVKGFYPSDIAVEWESNGQPENNYKTTPPVLDSDGSFFLYSKLTVDK | |
| | SRWQQGNVFSCSVMHEALHNHYTQKSLDKSTGKPTLYNVSLVMSDTAGTC | |
| | Y | |
| HEX-REGN3 | SDTGRPFVEMYSEIPEIIHMTEGRELVIPCRVTSPNITVTLKKFPLDTLI | 34 |
| (IgG4us) | PDGKRIIWDSRKGFIISNATYKEIGLLTCEATVNGHLYKTNYLTHRQTNT | |
| WT | IIDVVLSPSHGIELSVGEKLVLNCTARTELNVGIDFNWEYPSSKHQHKKL | |
| | VNRDLKTQSGSEMKKFLSTLTIDGVTRSDQGLYTCAASSGLMTKKNSTFV | |
| | RVHESKYGPPCPPCPAPGGGGPSVFLFPPKPKDTLMISRTPEVTCVVVDV | |
| | SQEDPEVQFNWYVDGVEVHNAKTKPREEQFNSTYRVVSVLTVLHQDWLNG | |
| | KEYKCKVSNKGLPSSIEKTISKAKGQPREPQVYTLPPSQEEMTKNQVSLT | |
| | CLVKGFYPSDIAVEWESNGQPENNYKTTPPVLDSDGSFFLYSRLTVDKSR | |
| | WQEGNVFSCSVMHEALHNHYTQKSLSLSLGKPTLYNVSLVMSDTAGTCY | |
| HEX-REGN3 | SDTGRPFVEMYSEIPEIIHMTEGRELVIPCRVTSPNITVTLKKFPLDTLI | 35 |
| (IgG4us) | PDGKRIIWDSRKGFIISNATYKEIGLLTCEATVNGHLYKTNYLTHRQTNT | |
| Mut1 | IIDVVLSPSHGIELSVGEKLVLNCTARTELNVGIDFNWEYPSSKHQHKKL | |
| | VNRDLKTQSGSEMKKFLSTLTIDGVTRSDQGLYTCAASSGLMTKKNSTFV | |
| | RVHESKYGPPCPPCPAPGGGGPSVFLFPPKPKDTLMISRTPEVTCVVVDV | |
| | SQEDPEVQFNWYVDGVEVHNAKTKPREEQFNSTYRVVSVLTVCHQDWLNG | |
| | KEYKCKVSNKGLPSSIEKTISKAKGQPREPQVYTLPPSQEEMTKNQVSLT | |
| | CLVKGFYPSDIAVEWESNGQPENNYKTTPPVLDSDGSFFLYSRLTVDKSR | |
| | WQEGNVFSCSVMHEALHNHYTQKSLDKSTGKPTLYNVSLIMSDTGGTCY | |

Hexameric VEGF antagonists were expressed in Expi293™ cells (ThermoFisher) by transient transfection, following the manufacturer's protocol.

Constructs were purified from the supernatant using a Mabselect Sure affinity resin-based column. Column equilibration buffer used was 50 mM Tris-HCl, 150 mM NaCl, pH7.5 for 5 column volumes (CV). Sterile filtered supernatant containing fusion protein was loaded over the pre-equilibrated column at a flow rate of 2.0 mL/min. After single step elution, the antibodies were neutralized, dialyzed into a final buffer of phosphate buffered saline (PBS) with 5% glycerol, aliquoted and stored at −80° C. Samples were further analyzed by size exclusion chromatography (SEC) to determine the presence of high or low molecular weight species relative to the species of interest.

### 8.1.3. VEGF Binding Assay

Binding affinity of VEGF antagonists to VEGF was evaluated using VEGF 165-Biotin. Each construct was diluted 1:5 from a starting concentration of 10 nM, except Hex-REGN3 (IgG1) Mut1 PVA/P329A, which was evaluated only at the highest concentration.

### 8.1.4. FcR Binding Assay

Binding affinity of VEGF antagonists to Fc receptors CD16 (FcγRIIIa), CD64 (FcγRI), and FcRn was evaluated using hCD16-biotin, hCD64-biotin, and hFcRn-β2M-biotin.

For FcγRIIIa and FcγRI binding assessments, each construct was diluted 1:5 from a starting concentration of 50 nM, except Hex-REGN3 (IgG1) Mut1 PVA/P329A, which was evaluated only at the highest concentration. For FcRn assessments, each construct was diluted 1:5 from a starting concentration of 200 nM, except Hex-REGN3 (IgG1) Mut1 PVA/P329A, which was evaluated only at the highest concentration.

### 8.2. Example 1. Multimeric Assembly of VEGF Antagonists

Hexameric VEGF antagonists were designed as described in Section 8.1.1. The constructs were produced and purified as described in Section 8.1.2. The multimerization of the constructs were assessed via SEC as described in Section 8.1.2 and non-reducing SDS-PAGE.

Constructs containing an L309C substitution relative to WT IgG1 (“Mut1”) displayed better hexameric assembly relative to their counterparts without the L309C substitution (“WT”) (FIG. 2). Construct 4, containing L309C and “LALAPG” substitutions, was associated with the highest level of hexameric assembly and minimal amount of low molecular weight products (FIG. 2).

The effect of Mut1 and LALAPG was further assessed by comparing the SEC profiles of the VEGF antagonists constructs Hex-REGN3 (IgG1) Mut2 LALAPG, Hex-REGN3 (IgG1) Mut1 LALAPG, and Hex-REGN3 (IgG1) Mut1-V2 PVA/P329A.

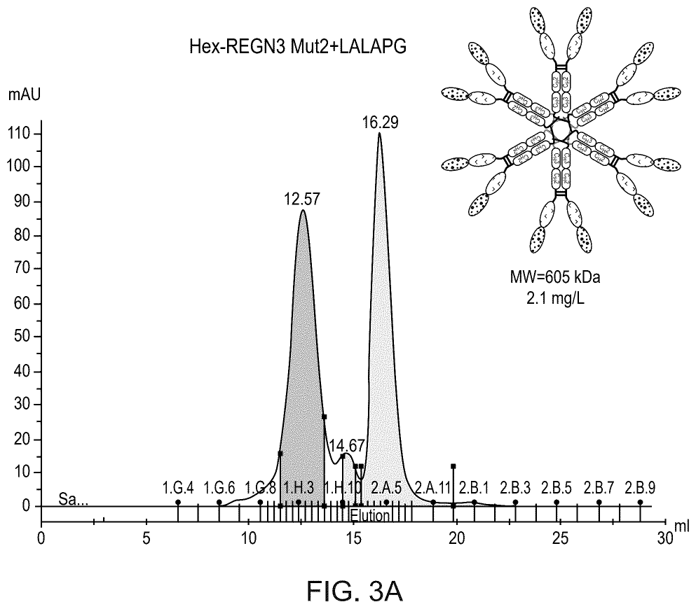

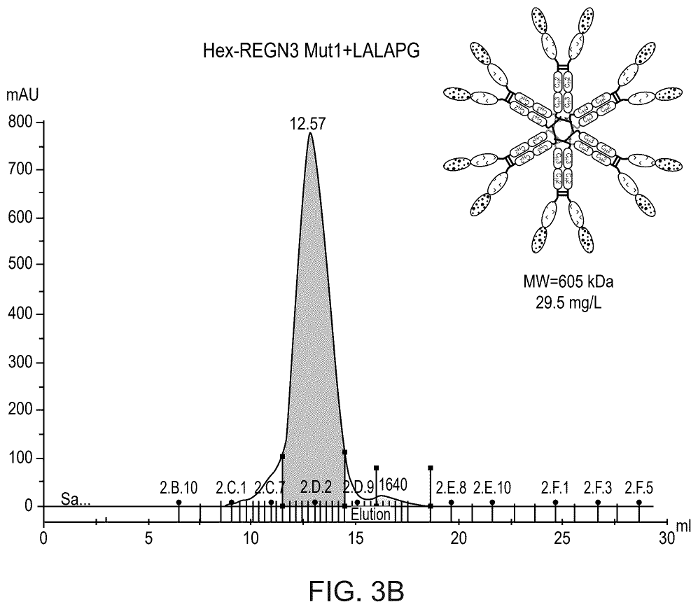

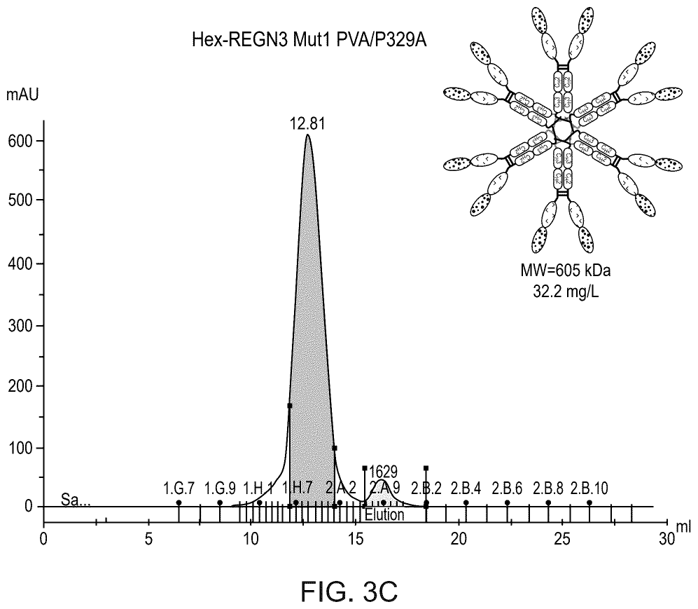

Hex-REGN3 (IgG1) Mut1 LALAPG (FIG. 3B) and Hex-REGN3 (IgG1) Mut1-V2 PVA/P329A (FIG. 3C) were associated with better overall yield and assembly relative to Hex-REGN3 (IgG1) Mut2 LALAPG (FIG. 3A).

### 8.3. Example 2. VEGF Binding of VEGF Antagonists

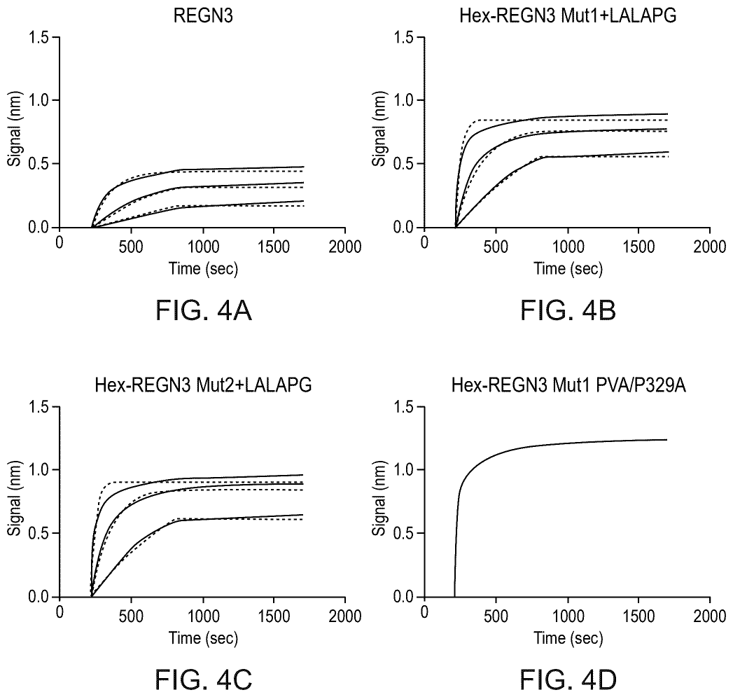

The affinity of the VEGF antagonists Hex-REGN3 (IgG1) Mut2 LALAPG, Hex-REGN3 (IgG1) Mut1 LALAPG, and Hex-REGN3 (IgG1) Mut1-V2 PVA/P329A to VEGF 165 was assessed as described in Section 8.1.3 and compared to the VEGF affinity of REGN3-Fc (“REGN3”; aflibercept, previously disclosed as SEQ ID NO:2 of WO2022/245739 A1). Hex-REGN3 (IgG1) Mut2 LALAPG, Hex-REGN3 (IgG1) Mut1 LALAPG, and Hex-REGN3 (IgG1) Mut1-V2 PVA/P329A displayed high affinity for VEGF₁₆₅ relative to REGN3-IgG Fc (FIG. 4A-4D).

### 8.4. Example 3. Fc Receptor Binding of VEGF Antagonists

Fc receptor binding affinity of the VEGF antagonists Hex-REGN3 (IgG1) Mut2 LALAPG, Hex-REGN3 (IgG1) Mut1 LALAPG, and Hex-REGN3 (IgG1) Mut1-V2 PVA/P329A was assessed as described in Section 8.1.3 and compared to the Fc receptor binding affinity of REGN3-Fc (“REGN3”; aflibercept).

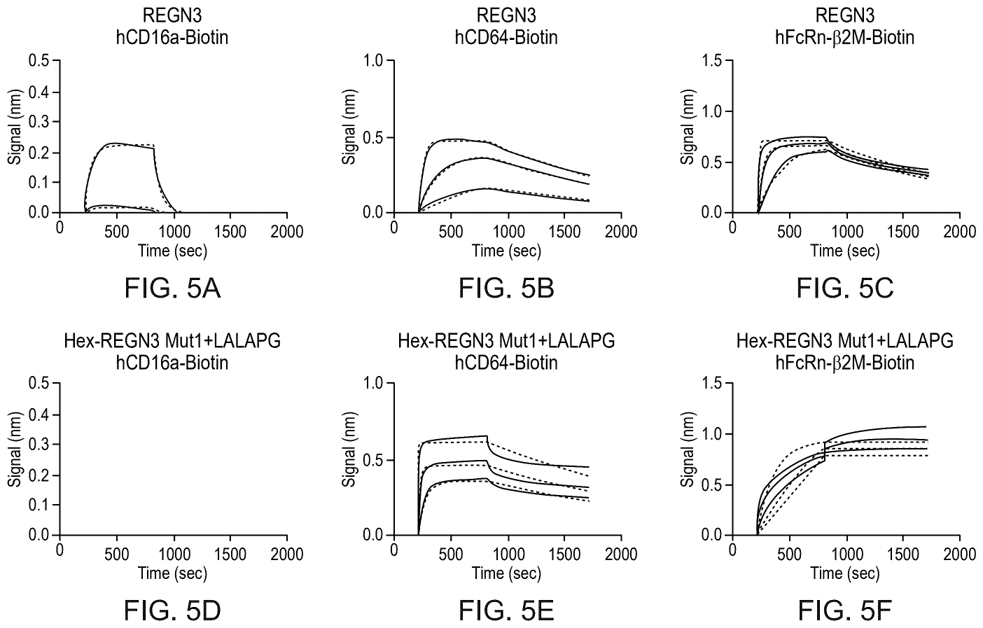

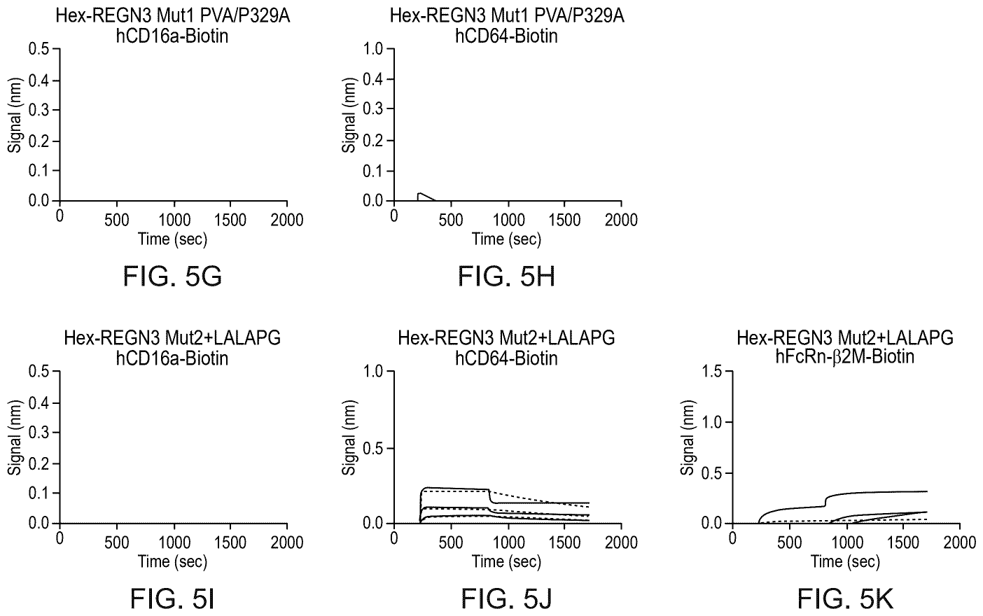

REGN3 showed binding to all of CD16a, CD64, and FcRn. Hex-REGN3 (IgG1) Mut2 LALAPG, Hex-REGN3 (IgG1) Mut1 LALAPG, and Hex-REGN3 (IgG1) Mut1-V2 PVA/P329A showed no binding to CD16a (FIGS. 5D, 5G, and 5I, respectively). Hex-REGN3 (IgG1) Mut2 LALAPG and Hex-REGN3 (IgG1) Mut1 LALAPG both showed low binding to CD64 and FcRn relative to REGN3, while Hex-REGN3 (IgG1) Mut1 PVA/P329A showed virtually no binding to CD64. These results are further summarized in Table E4.

TABLE E4

| | CD16a (FcGRIIIa) | | | CD64 (FcGRI) | | | FcRn | | |
|---|---|---|---|---|---|---|---|---|---|
| | KD | kₒₙ | k_{dis} | KD | kₒₙ | k_{dis} | KD | kₒₙ | k_{dis} |
| Construct | (M) | (1/Ms) | (1/s) | (M) | (1/Ms) | (1/s) | (M) | (1/Ms) | (1/s) |
| REGN3 | 2.76E−07 | 6.11E+04 | 1.69E−02 | 1.37E−09 | 5.53E+05 | 7.56E−04 | 1.28E−09 | 5.35E+05 | 6.85E−04 |
| Hex-REGN3 | N.B. | N.B. | N.B. | 6.38E−11 | 8.01E+06 | 5.11E−04 | <1.0E−12 | 3.10E+04 | <1.0E−07 |
| (IgG1) Mut1 | | | | | | | | | |
| LALAPG | | | | | | | | | |
| Hex-REGN3 | N.B. | N.B. | N.B. | P.F. | P.F. | P.F. | P.F. | P.F. | P.F. |
| (IgG1) Mut2 | | | | | | | | | |
| LALAPG | | | | | | | | | |
| Hex-REGN3 | N.B. | N.B. | N.B. | N.B. | N.B. | N.B. | N.D. | N.D. | N.D. |
| (IgG1) Mut1- | | | | | | | | | |
| V2 PVA/P329A | | | | | | | | | |

N.B.: No binding was detected; P.F.: Poor fit (low binding); N.D.: Not yet determined.

### 8.5. Example 4. VEGF Binding Saturation of Hex-REGN3 Constructs

VEGF binding saturation of the REGN3 (IgG1) Mut1 LALAPG construct was assessed with mass spectrophotometry.

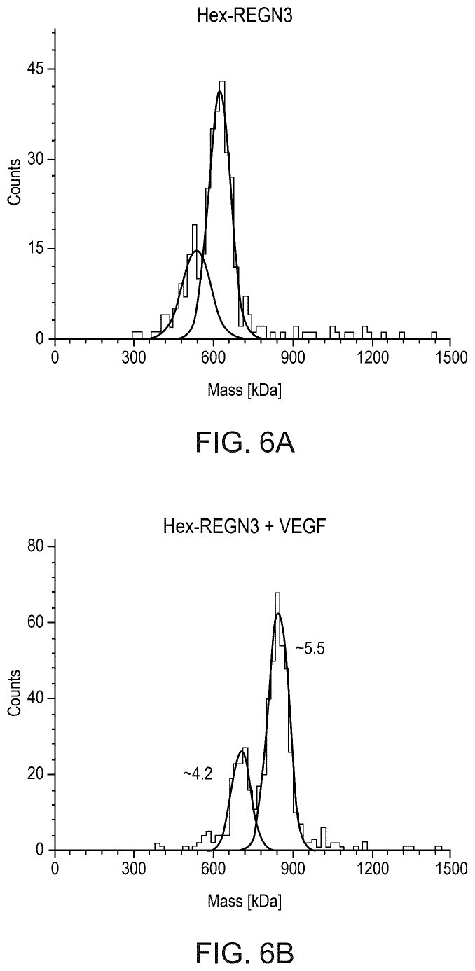

The molecular weight of free Hex-REGN3 (e.g., a Hex-REGN3 that is not bound to VEGF) was estimated to be 605 kDa. In the absence of VEGF, the spectrophotometry revealed two peaks: a small peak that corresponded to a molecule of approximately 538 kDa and a larger peak that corresponded to a molecule of approximately 625 kDa (FIG. 6A). Thus, most of the Hex-REGN3 in the evaluated sample was hexameric while a small portion of the sample was pentameric. The presence of VEGF caused a rightward shift in the peaks. This time, the smaller peak corresponded to a molecule of approximately 707 kDa and the larger peak corresponded to a molecule of approximately 846 kDa (FIG. 6B). In other words, each pentameric HexREGN3 was bound to an average of 4.2 VEGF molecules, whereas each hexameric Hex-REGN3 was bound to an average of 5.5 VEGF molecules.

### 8.6. Example 5. Stability of the IgG1-T-Hex Constructs

“Wildtype” IgG1-tailpiece (IgG1-T-Hex (WT)) constructs were designed which lacked any binding domain, comprising two polypeptides, each having, in N- to C-terminal order, a hinge domain, an IgG1 CH2 domain, an IgG1 CH3 domain, and an IgM tailpiece (as depicted in FIG. 1A). The IgG CH3 domain included the amino acid substitutions S442D, L443K, and P445T (EU numbering). Fusion of the IgM tailpiece sequences enabled multimerization, resulting in IgG1-tailpiece hexamers (as depicted in FIG. 1B). “Mutated” IgG1-tailpiece (IgG1-T-Hex (Mut1)) constructs were designed similarly, and further comprised an additional amino acid substitution in the IgG CH2 domain (L309C, EU numbering) to enable disulfide connections between the CH2 domains of the two polypeptides, and two amino acid substitutions within the tailpiece (V5671 and A572G, EU numbering). All constructs were expressed in Expi293™ cells (ThermoFisher) by transient transfection, following the manufacturer's protocol.

Constructs were purified from the supernatant using a Mabselect Sure affinity resin based column. Column equilibration buffer used was 50 mM Tris-HCl, 150 mM NaCl, pH7.5 for 5 column volumes (CV). Sterile filtered supernatant containing fusion protein was loaded over the pre-equilibrated column at a flow rate of 2.0 mL/min. After single step elution, the molecules were neutralized, dialyzed into a final buffer of phosphate buffered saline (PBS) with 5% glycerol, aliquoted and stored at −80° C. Samples were further analyzed by size exclusion chromatography (SEC) to determine the presence of high or low molecular weight species relative to the species of interest.

Sequences of the IgG1-T-Hex (WT) and IgG1-T-Hex (Mut1) used in these studies are provided in Table E5, below.

TABLE E5
Hexameric IgG1-Tailpiece Sequences

| | | SEQ ID |
|---|---|---|
| Name | Sequence | NO: |
| IgG1-T- | EPKSCDKTHTCPPCPAPELLGGPSV | 91 |
| Hex(WT) | FLFPPKPKDTLMISRTPEVTCVVVD | |
| | VSHEDPEVKFNWYVDGVEVHNAKTK | |
| | PREEQYNSTYRVVSVLTVLHQDWLN | |
| | GKEYKCKVSNKALPAPIEKTISKAK | |
| | GQPREPQVYTLPPSRDELTKNQVSL | |
| | TCLVKGFYPSDIAVEWESNGQPENN | |
| | YKTTPPVLDSDGSFFLYSKLTVDKS | |
| | RWQQGNVFSCSVMHEALHNHYTQKS | |
| | LDKSTGKPTLYNVSLVMSDTAGTCY | |
| IgG1-T- | EPKSCDKTHTCPPCPAPELLGGPSV | 6 |
| Hex(Mut1) | FLFPPKPKDTLMISRTPEVTCVVVD | |
| | VSHEDPEVKFNWYVDGVEVHNAKTK | |
| | PREEQYNSTYRVVSVLTVCHQDWLN | |
| | GKEYKCKVSNKALPAPIEKTISKAK | |
| | GQPREPQVYTLPPSREEMTKNQVSL | |
| | TCLVKGFYPSDIAVEWESNGQPENN | |
| | YKTTPPVLDSDGSFFLYSKLTVDKS | |
| | RWQQGNVFSCSVMHEALHNHYTQKS | |
| | LDKSTGKPTLYNVSLIMSDTGGTCY | |

The stability of the IgG1-T-Hex (WT) and IgG1-T-Hex (Mut1) constructs were assessed via SEC as well as with reducing and non-reducing SDS-PAGE either immediately after purification or after storage of the purified samples at 4° C. for over 30 days.

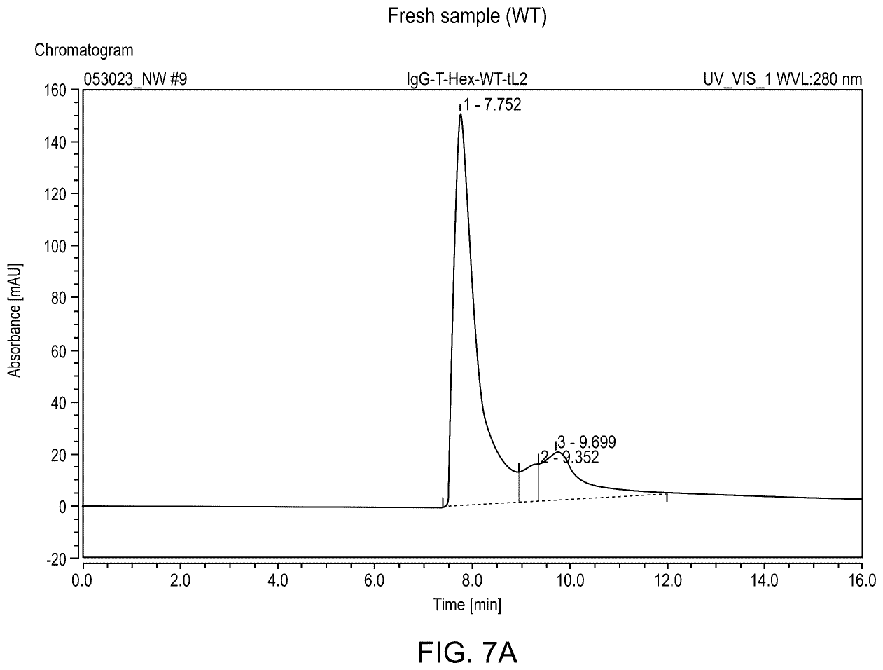

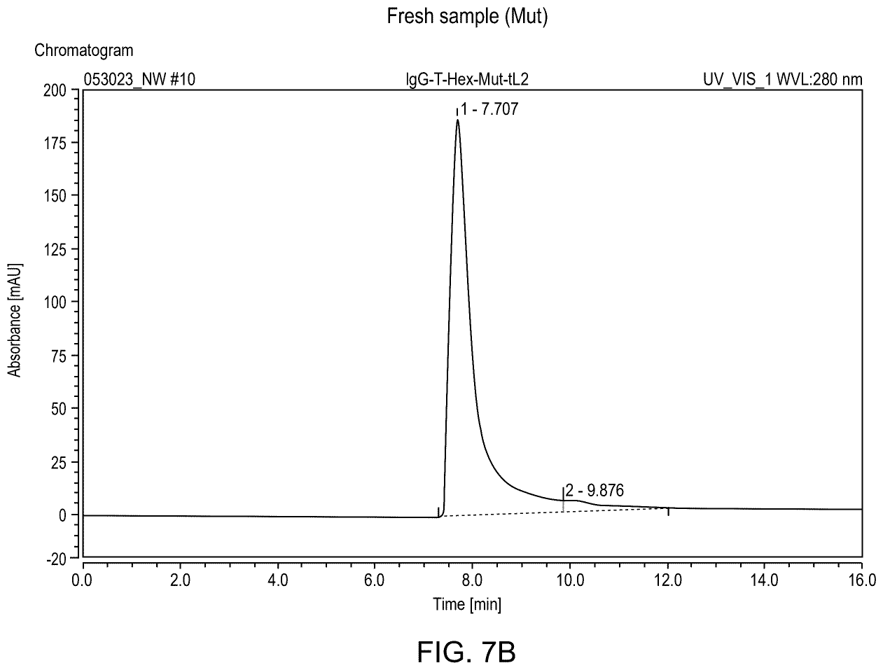

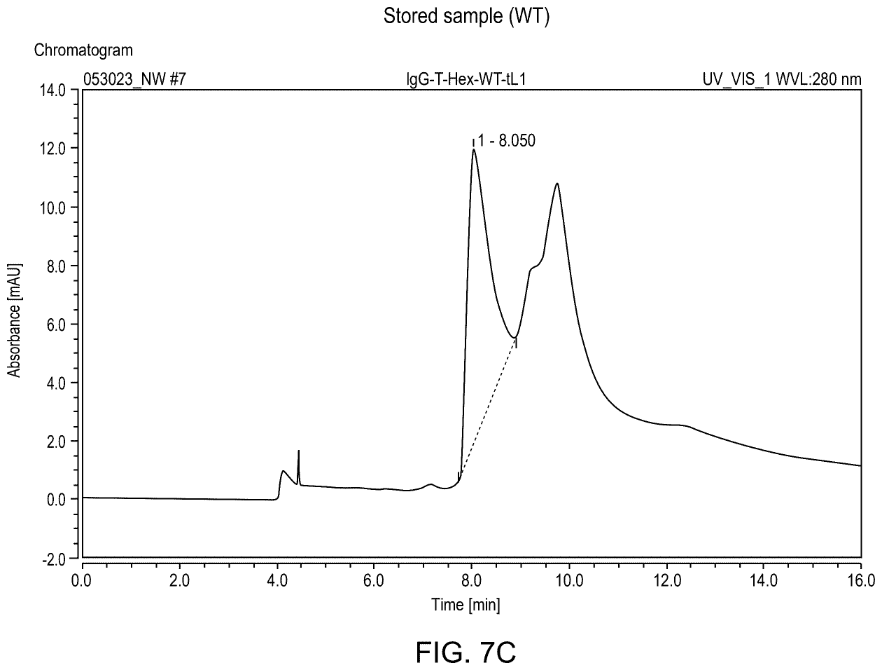

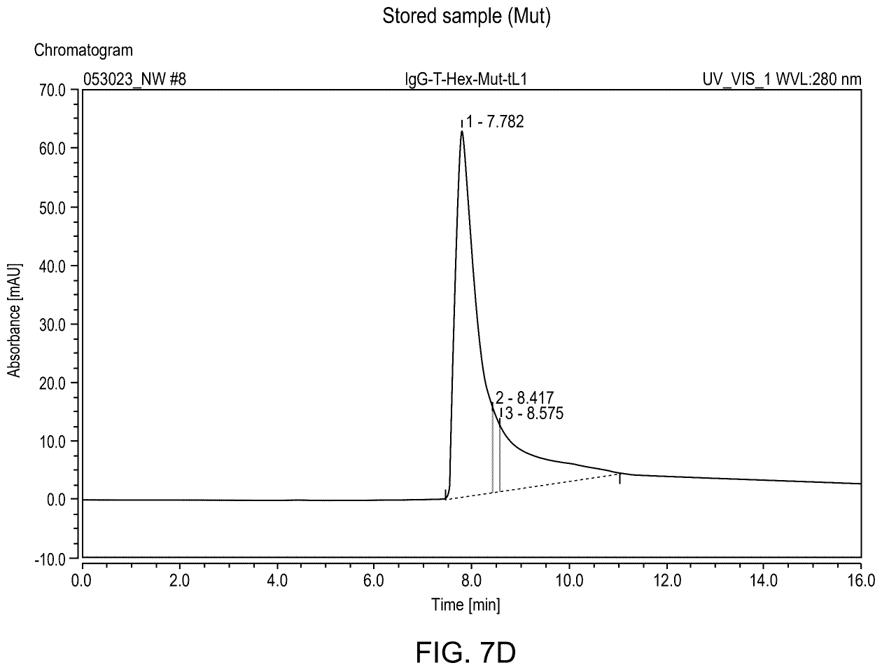

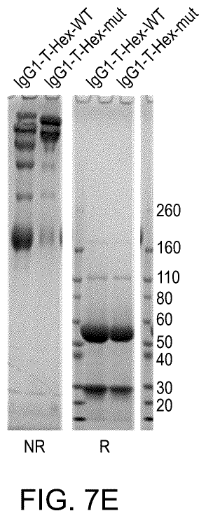

SEC analysis of freshly prepared IgG1-T-Hex (WT) and IgG1-T-Hex (Mut1) samples showed robust peaks for both constructs (FIGS. 7A-7B). IgG1-T-Hex (Mut1) did not display any additional peaks (FIG. 7B), whereas a smaller peak corresponding to lower molecular weight species was observed with IgG1-T-Hex (WT) (FIG. 7A). SEC analysis of the same samples after over 30 days of storage at 4° C., revealed a pronounced dissociation of the IgG1-T-Hex (WT) (FIG. 7C), whereas IgG1-T-Hex (Mut1) sample displayed minimal dissociation (FIG. 7D).

Similar results were observed with SDS-PAGE analysis of IgG1-T-Hex (WT) and IgG1-T-Hex (Mut1) constructs that were stored for over 30 days at 4° C. Lanes for both constructs displayed bands of similar sizes with SDS-PAGE under reducing (R) conditions, indicating the production efficiency for WT and Mut1 constructs were comparable (FIG. 2E). However, the bands that corresponded to hexameric constructs were more pronounced for the IgG1-T-Hex (Mut1) with SDS-PAGE under non-reducing (NR) conditions, whereas IgG1-T-Hex (WT) displayed relatively thicker bands that correspond to lower molecular weight species (FIG. 7E), indicating decreased efficiency of disulfide bond formation and corresponding dissociation of IgG1-T-Hex (WT) relative to IgG1-T-Hex (Mut1).

Taken together, these results suggest that IgG1-T-Hex (Mut1) is able to form homogenous and stable hexamers. In contrast, IgG1-T-Hex (WT) hexamers did not tolerate extended storage and dissociated at a higher rate.

### 8.7. Example 6. FcRn Binding Affinities of IgG1-T-Hex Constructs with Fab Domains

IgG1-T-Hex-WT and IgG1-T-Hex-Mut1 constructs comprising Fab domains were designed, where a Fab domain of an antibody was fused directly to the hinge domains, designated “Fab-IgG1-T-Hex-WT” and “Fab-IgG1-T-Hex-Mut1”, respectively. All constructs were expressed in Expi293™ cells (ThermoFisher) by transient transfection, following the manufacturer's protocol.

Constructs were purified from the supernatant using a Mabselect Sure affinity resin based column. Column equilibration buffer used was 50 mM Tris-HCl, 150 mM NaCl, pH7.5 for 5 column volumes (CV). Sterile filtered supernatant containing fusion protein was loaded over the pre-equilibrated column at a flow rate of 2.0 mL/min. After single step elution, the molecules were neutralized, dialyzed into a final buffer of phosphate buffered saline (PBS) with 5% glycerol, aliquoted and stored at −80° C. Samples were further analyzed by size exclusion chromatography (SEC) to determine the presence of high or low molecular weight species relative to the species of interest.

To determine the degree to which IgG1-T-Hex constructs bind to FcRn, the binding affinities of Fab-IgG1-T-Hex-WT and Fab-IgG1-T-Hex-Mut1 were assessed, along with the parental antibody from which the Fab domains were derived, via Biacore analysis at 25° C., pH 6.0. The results of this analysis are summarized in Table E6 below. An antibody having a high affinity to FcRn was included as a control (Ctrl).

TABLE E6

| Antibody Injected | hFcRn Capture | Antibody | |
|---|---|---|---|
| (1 μM) | (RU) | Bound (RU) | K_{D} (M) |
| Parental (IgG1) | 123.8 ± 1.4 | 16.7 | 4.60E−06 |
| Ctrl IgG1 | 120.2 ± 0.7 | 83.7 | 2.07E−07 |
| Fab-IgG1-T-Hex-WT | 114.4 ± 1.4 | 18.9 | 3.60E−06 |
| REGNFab14287-IgG1- | 112.2 ± 0.6 | 7.1 | 1.2E−05 |
| T-Hex-Mut1 | | | |

The parental antibody and the WT hexameric construct Fab-IgG1-T-Hex-WT displayed similar levels of binding affinity to FcRn, while Fab-IgG1-T-Hex-Mut1 had a moderately reduced binding affinity to FcRn relative to the parental antibody and Fab-IgG1-T-Hex-WT.

### 8.8. Example 7. In Vivo Pharmacokinetic Study of VEGF Antagonist Hex-REGN3 (IgG1) Mut1-V2 PVA/P329A

Hex-REGN3 (IgG1) Mut1-V2 PVA/P329A (SEQ ID NO:18) was conjugated to Alexa Fluor™ 488 dyes at two different VEGF antagonist: dye ratios. These compositions (termed “240419E” for the first ratio and “240419F” for the second ratio) were administered via bilateral IVT injection to both eyes of four male NZW rabbits (two rabbits for each ratio) at 60 L/eye as follows:
240419E—concentration 4.23 mg/ml, endotoxin 7.03EU/ml
240419F—concentration 2.08 mg/ml, endotoxin 4.58EU/ml

Baseline measurements were taken of intraocular pressure (IOP) and fluorescence prior to administration, followed by measurements taken immediately after IVT injection (day 0), 10 and 30 minutes after injection, then again on days 5, 8, 15, 25, and 29. Fluorescence was measured by ocular fluorophotometry with a Fluorotron™ ocular scanning fluorophotometer.

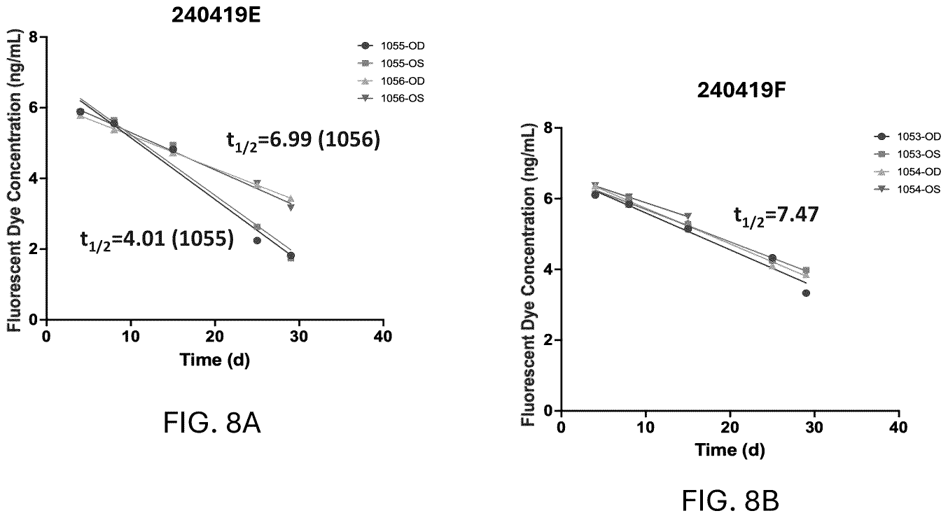

Fluorescent dye concentration for each animal, measured in each eye, is shown in FIGS. 8A and 8B. The half-life of 240419E was about 7.0 days for one rabbit (1056) and about 4.0 days for the other (1055). The half-life of 240419F was about 7.5 days for both rabbits (1053 and 1054). Previous, similar experiments performed showed REGN3 (aflibercept) as having a half-life of about 4.8 days. Overall, these results suggest that Hex-REGN3 (IgG1) Mut1-V2 PVA/P329A has increased in vivo half-life compared to REGN3.

1. A VEGF antagonist comprising five or more dimers, each dimer comprising two polypeptide chains, each polypeptide chain comprising, in N- to C-terminal orientation:
(a) a VEGF binding domain comprising:
(i) an Ig-like domain 2 of VEGFR1 comprising an amino acid sequence having at least 95% sequence identity to SEQ ID NO: 13; and
(ii) an Ig-like domain 3 of VEGFR2 comprising an amino acid sequence having at least 95% sequence identity to SEQ ID NO: 15 or SEQ ID NO:50; and
(b) a multimerization domain comprising, in N- to C-terminal orientation:
(i) an IgG Fc domain having at least 90% sequence identity to the amino acid sequence of SEQ ID NO:1; and
(ii) an IgM tailpiece having at least 90% sequence identity to the amino acid sequence of SEQ ID NO:2.

2. The VEGF antagonist of claim 1, wherein the IgM tailpiece comprises the amino acid sequence of SEQ ID NO:3 or SEQ ID NO:4.

3. (canceled)

4. The VEGF antagonist of claim 1, wherein the IgG Fc domain has at least 98% sequence identity to the amino acid sequence of any one of SEQ ID NOs: 5-12 and 52-58.

5. (canceled)

6. The VEGF antagonist of claim 1, wherein the IgG Fc domain comprises the amino acid cysteine at position 309, as defined by EU numbering.

7. The VEGF antagonist of claim 1, wherein the IgG Fc domain comprises the amino acid isoleucine at position 253 or the amino acid cysteine at position 253, as defined by EU numbering.

8. (canceled)

9. The VEGF antagonist of claim 1, wherein the IgG Fc domain comprises the amino acids aspartic acid at position 359 and leucine at position 361, as defined by EU numbering.

10. The VEGF antagonist of claim 1, wherein the IgG Fc domain comprises the amino acids serine at position 442, leucine at position 443, and proline at position 445, as defined by EU numbering.

11. The VEGF antagonist of claim 1, wherein the IgG Fc domain comprises one or more mutations that decrease affinity for FcRn as compared to an IgG Fc domain having the amino acid sequence of SEQ ID NO:1.

12. (canceled)

13. The VEGF antagonist of claim 1, wherein the IgG Fc domain comprises one or more mutations that decrease affinity for a FcγR as compared to an IgG Fc domain having the amino acid sequence of SEQ ID NO: 1.

14.-17. (canceled)

18. A VEGF antagonist comprising five or more dimers, each dimer comprising two polypeptides, each polypeptide comprising, in N- to C-terminal orientation:
(a) a VEGF binding domain comprising:
(i) an Ig-like domain 2 of VEGFR1 comprising an amino acid sequence having at least 95% sequence identity to SEQ ID NO:13; and
(ii) an Ig-like domain 3 of VEGFR2 comprising an amino acid sequence having at least 95% sequence identity to SEQ ID NO: 15 or SEQ ID NO:50; and
(b) a multimerization domain having at least 95% identity to the amino acid sequence of SEQ ID NO: 5, SEQ ID NO:6, SEQ ID NO:7, SEQ ID NO:8, SEQ ID NO:9, SEQ ID NO: 10, or SEQ ID NO: 11.

19. The VEGF antagonist of claim 18, wherein the multimerization domain has at least 99% identity to the amino acid sequence of SEQ ID NO:5, SEQ ID NO:6, SEQ ID NO:7, SEQ ID NO:8, SEQ ID NO:9, SEQ ID NO: 10, or SEQ ID NO:11.

20. The VEGF antagonist of claim 18, wherein the multimerization domain comprises the amino acid sequence of SEQ ID NO:5, SEQ ID NO:6, SEQ ID NO:7, SEQ ID NO:8, SEQ ID NO:9, SEQ ID NO: 10, or SEQ ID NO: 11.

21.-38. (canceled)

39. The VEGF antagonist of claim 1, wherein the Ig-like domain 2 of VEGFR1 has at least 98% sequence identity to SEQ ID NO:13 or SEQ ID NO: 14.

40.-42. (canceled)

43. The VEGF antagonist of claim 1, wherein the Ig-like domain 3 of VEGFR2 has at least 98% sequence identity to SEQ ID NO:15 or SEQ ID NO:50.

44. (canceled)

45. The VEGF antagonist of claim 1, wherein the VEGF binding domain comprises an amino acid sequence having at least 95% identity to SEQ ID NO: 16.

46. (canceled)

47. (canceled)

48. A VEGF antagonist comprising five or more dimers, each dimer comprising two polypeptides, each comprising an amino acid sequence having at least 95% sequence identity to the amino acid sequence of SEQ ID NO: 18, SEQ ID NO: 19, or SEQ ID NO:20.

49. The VEGF antagonist of claim 48, wherein each polypeptide comprises an amino acid sequence having at least 99% identity to SEQ ID NO: 18, SEQ ID NO: 19, or SEQ ID NO:20.

50. The VEGF antagonist of claim 48, wherein each polypeptide comprises the amino acid sequence of SEQ ID NO: 18, SEQ ID NO: 19, or SEQ ID NO: 20.

51.-59. (canceled)

60. The VEGF antagonist of claim 1, wherein the VEGF antagonist comprises six dimers.

61. A VEGF antagonist comprising:
(a) a VEGF binding domain; and
(b) a multimerization domain comprising:
(i) a chimeric IgG Fc domain comprising an amino acid sequence that has at least 90% identity to SEQ ID NO: 1, provided that the chimeric IgG Fc domain has the sequence P-V-A-absent at amino acids 233 to 236 and the amino acid substitution P329A as compared to SEQ ID NO:1, as defined by EU numbering; and
(ii) an IgM tailpiece having at least 80% sequence identity to the amino acid sequence of SEQ ID NO:2.

62. The VEGF antagonist of claim 61, wherein the VEGF binding domain is a Fab or component thereof or an scFv.

63. (canceled)

64. The VEGF antagonist of claim 61, wherein the VEGF binding domain comprises a VEGF receptor or portion thereof.

65. The VEGF antagonist of claim 64, wherein the VEGF binding domain comprises, in N- to C-terminal orientation, an Ig-like domain 2 of VEGFR1 and an Ig-like domain 3 of VEGFR2.

66. (canceled)

67. (canceled)

68. The VEGF antagonist of claim 61, wherein the VEGF antagonist comprises a dimer comprising two polypeptides, each polypeptide comprising the VEGF binding domain, the chimeric IgG Fc domain, and the IgM tailpiece.

69. The VEGF antagonist of claim 68, wherein the VEGF antagonist comprises five or more dimers.

70. A composition comprising a population of VEGF antagonists according to claim 1.

71. (canceled)

72. (canceled)

73. The composition of claim 70, wherein at least 80% of the population of VEGF antagonists are molecules comprising five or more dimers.

74.-76. (canceled)

77. The composition of claim 70, which is a pharmaceutical composition comprising one or more pharmaceutically acceptable carriers or excipients.

78. A nucleic acid or plurality of nucleic acids encoding the polypeptides of the VEGF antagonist of claim 1.

79. A host cell engineered to express the nucleic acid(s) of claim 78.

80. A method of producing a VEGF antagonist comprising culturing the host cell of claim 79 under conditions sufficient to express the polypeptides.

81. (canceled)

82. A method of inhibiting VEGF receptor activity in a cell comprising contacting the cell with the VEGF antagonist of claim 1.

83. (canceled)

84. A method for treating an angiogenic eye disorder in a subject comprising administering to the subject the VEGF antagonist of claim 1.

85. (canceled)

86. (canceled)


SEQUENCE LISTING (101 sequences)

SEQ ID NO: 1 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 18 aa, synthetic construct
  VARIANT 9: note = Any amino acid
  VARIANT 14: note = Any amino acid
PTLYNVSLXMSDTXGTCY

SEQ ID NO: 3 - protein, 18 aa, unidentified
PTLYNVSLVMSDTAGTCY

SEQ ID NO: 4 - protein, 18 aa, synthetic construct
PTLYNVSLIMSDTGGTCY

SEQ ID NO: 5 - protein, 245 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLDKSTGKPTLYNVSLVMSDTAGTCY

SEQ ID NO: 6 - protein, 250 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVCHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLDKSTGKPTLYNVSLIMSDTGGTCY

SEQ ID NO: 7 - protein, 245 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVCHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLDKSTGKPTLYNVSLIMSDTGGTCY

SEQ ID NO: 8 - protein, 245 aa, synthetic construct
DKTHTCPPCPAPELLGGPSVFLFPPKPKDTLMCSRTPEVTCVVVDVSHEAPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 9 - protein, 245 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVCHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLDKSTGKPTLYNVSLVMSDTAGTCY

SEQ ID NO: 10 - protein, 244 aa, synthetic construct
DKTHTCPPCPAPPVAGPSVFLFPPKPKDTLMISRTPEVTCVVVDVSHEDPEVKFNWYVDGVEVHNAKTKPREEQYNSTYRVVSVLTVCHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLDKSTGKPTLYNVSLVMSDTAGTCY

SEQ ID NO: 11 - protein, 245 aa, synthetic construct
DKTHTCPPCPAPEAAGGPSVFLFPPKPKDTLMCSRTPEVTCVVVDVSHEAPEVKFNWYVDGVEVHNAKTKPREEQYNSTYRVVSVLTVLHQDWLNGKEYKCKVSNKALGAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 12 - protein, 246 aa, synthetic construct
ESKYGPPCPPCPAPGGGGPSVFLFPPKPKDTLMISRTPEVTCVVVDVSQEDPEVQFNWYVDGVEVHNAKTKPREEQFNSTYRVVSVLTVCHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLDKSTGKPTLYNVSLVMSDTAGTCY

SEQ ID NO: 13 - protein, 64 aa, Homo sapiens
GRELVIPCRVTSPNITVTLKKFPLDTLIPDGKRIIWDSRKGFIISNATYKEIGLLTCEATVNGH

SEQ ID NO: 14 - protein, 103 aa, Homo sapiens
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIID

SEQ ID NO: 15 - protein, 102 aa, Homo sapiens
VVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 16 - protein, 205 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 17 - protein, 450 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVCHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLDKSTGKPTLYNVSLVMSDTAGTCY

SEQ ID NO: 18 - protein, 449 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPPVAGPSVFLFPPKPKDTLMISRTPEVTCVVVDVSHEDPEVKFNWYVDGVEVHNAKTKPREEQYNSTYRVVSVLTVCHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLDKSTGKPTLYNVSLVMSDTAGTCY

SEQ ID NO: 19 - protein, 450 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMCSRTPEVTCVVVDVSHEAPEVKFNWYVDGVEVHNAKTKPREEQYNSTYRVVSVLTVLHQDWLNGKEYKCKVSNKALGAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 20 - protein, 451 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKESKYGPPCPPCPAPGGGGPSVFLFPPKPKDTLMISRTPEVTCVVVDVSQEDPEVQFNWYVDGVEVHNAKTKPREEQFNSTYRVVSVLTVCHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLDKSTGKPTLYNVSLVMSDTAGTCY

SEQ ID NO: 21 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 22 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 23 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 29 - protein, 235 aa, synthetic construct
DKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 228 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 32 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 245 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVCHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLDKSTGKPTLYNVSLVMSDTAGTCY

SEQ ID NO: 34 - protein, 449 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHESKYGPPCPPCPAPGGGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPTLYNVSLVMSDTAGTCY

SEQ ID NO: 35 - protein, 449 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHESKYGPPCPPCPAPGGGGPSVFLFPPKPKDTLMISRTPEVTCVVVDVSQEDPEVQFNWYVDGVEVHNAKTKPREEQFNSTYRVVSVLTVCHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLDKSTGKPTLYNVSLIMSDTGGTCY

SEQ ID NO: 36 - protein, 450 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 37 - protein, 450 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVCHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLDKSTGKPTLYNVSLIMSDTGGTCY

SEQ ID NO: 38 - protein, 392 aa, Mus sp.
MTDRQTDTAPSPSAHLLAGGLPTVDAAASREEPKPAPGGGVEGVGARGIARKLFVQLLGSSRSVVAVVCAAGDKPIGAGRSASSGLEKPGPEKRGEEEKEEERGPQWALGSQEPSSWTGEAAVCADSAPAARAPQAPARASVPEGRGARQGAQESGLPRSPSRRGSASRAGPGRASETMNFLLSWVHWTLALLLYLHHAKWSQAAPTTEGEQKSHEVIKFMDVYQRSYCRPIETLVDIFQEYPDEIEYIFKPSCVPLMRCAGCCNDEALECVPTSESNITMQIMRIKPHQSQHIGEMSFLQHSRCECRPKKDRTKPEKKSVRGKGKGQKRKRKKSRFKSWSVHCEPCSERRKHLFVQDPQTCKCSCKNTDSRCKARQLELNERTCRCDKPRR

SEQ ID NO: 39 - protein, 395 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 40 - protein, 1338 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELITLTCTCVAATLFWLLLTLFIRKMKRSSSEIKTDYLSIIMDPDEVPLDEQCERLPYDASKWEFARERLKLGKSLGRGAFGKVVQASAFGIKKSPTCRTVAVKMLKEGATASEYKALMTELKILTHIGHHLNVVNLLGACTKQGGPLMVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 41 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 42 - protein, 126 aa, synthetic construct
QVQLVQSGAEVKKPGASVKVSCKASGGTFSSYAISWVRQAPGQGLEWMGGFDPEDGETIYAQKFQGRVTMTEDTSTDTAYMELSSLRSEDTAVYYCATGRSMVRGVIIPFNGMDVWGQGTTVTVSS

SEQ ID NO: 43 - protein, 107 aa, synthetic construct
DIRMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 44 - protein, 120 aa, synthetic construct
EVQLVESGGGLVQPGGSLRLSCAASGFTISDYWIHWVRQAPGKGLEWVAGITPAGGYTRYADSVKGRFTISADTSKNTAYLQMRSLRAEDTAVYYCARFVFFLPYAMDYWGQGTLVTVSS

SEQ ID NO: 45 - protein, 107 aa, synthetic construct
DIQMTQSPSSLSASVGDRVTITCRASQDVSTAVAWYQQKPGKAPKLLIYSASFLYSGVPSRFSGSGSGTDFTLTISSLQPEDAATYYCQQGYGAPFTFGQGTKVEIK

SEQ ID NO: 46 - protein, 120 aa, synthetic construct
EEQLVEEGGGLVQPGESLELSCAASGFEISDYWIHWVRQEPGEGLEWVAGITPAGGYEYYADSVEGRFTISADTSENTAYLQMNELRAEDTAVYYCARFVFFLPYAMDYWGQGELVTVSS

SEQ ID NO: 47 - protein, 107 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQGYGNPFTFGQGTKVEIK

SEQ ID NO: 48 - protein, 107 aa, synthetic construct
DIQMTQSPESLSASVGDEVTITCRASQDVSTAVAWYQQKPGKAPKLLIYSASFLYSGVPSRFSGSGSGTDFTLTISSLQPEDAATYYCQQGYGAPFTFGQGTKVEIK

SEQ ID NO: 49 - protein, 107 aa, synthetic construct
DIQMTQSPSSLSASVGDEVTITCRASQDVSTAVAWYQQKPGEAPKLLIYSASFLYSGVPSRFSGSGSGTDFTLTIESLQPEDAATYYCQQGYGAPFTFGQGTKVEIK

SEQ ID NO: 50 - protein, 95 aa, Homo sapiens
VVLSPSHGIELSVGEKLVLNCTARTELNVGIDFNWEYPSSKHQHKKLVNRDLKTQSGSEMKKFLSTLTIDGVTRSDQGLYTCAASSGLMTKKNST

SEQ ID NO: 51 - protein, 245 aa, synthetic construct; identical to SEQ ID NO: 5

SEQ ID NO: 52 - protein, 250 aa, synthetic construct; identical to SEQ ID NO: 6

SEQ ID NO: 53 - protein, 250 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVCHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLDKSTGKPTLYNVSLVMSDTAGTCY

SEQ ID NO: 54 - protein, 250 aa, synthetic construct
EPKSCDKTHTCPPCPAPELLGGPSVFLFPPKPKDTLMCSRTPEVTCVVVDVSHEAPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 55 - protein, 250 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVCHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLDKSTGKPTLYNVSLVMSDTAGTCY

SEQ ID NO: 56 - protein, 249 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVCHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLDKSTGKPTLYNVSLVMSDTAGTCY

SEQ ID NO: 57 - protein, 250 aa, synthetic construct
EPKSCDKTHTCPPCPAPEAAGGPSVFLFPPKPKDTLMCSRTPEVTCVVVDVSHEAPEVKFNWYVDGVEVHNAKTKPREEQYNSTYRVVSVLTVLHQDWLNGKEYKCKVSNKALGAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 58 - protein, 241 aa, synthetic construct
PPCPPCPAPGGGGPSVFLFPPKPKDTLMISRTPEVTCVVVDVSQEDPEVQFNWYVDGVEVHNAKTKPREEQFNSTYRVVSVLTVCHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLDKSTGKPTLYNVSLVMSDTAGTCY

SEQ ID NO: 59 - protein, 107 aa, synthetic construct
DIQMTQSPESLSASVGDEVTITCRASQDVSTAVAWYQQKPGEAPKLLIYSASFLYSGVPSRFSGSGSGTDFTLTISSLQPEDAATYYCQQGYGAPFTFGQGTKVEIK

SEQ ID NO: 60 - protein, 120 aa, synthetic construct
EEQLVEEGGGLVQPGESLRLSCAASGFEISDYWIHWVRQEPGEGLEWVAGITPAGGYEYYADSVEGRFTISADTSENTAYLQMNELRAEDTAVYYCARFVFFLPYAMDYWGQGELVTVSS

SEQ ID NO: 61 - protein, 107 aa, synthetic construct
DIQMTQSPSSLSASVGDEVTITCRASQDVSTAVAWYQQKPGKAPKLLIYSASFLYSGVPSRFSGSGSGTDFTLTISSLQPEDAATYYCQQGYGAPFTFGQGTKVEIK

SEQ ID NO: 62 - protein, 120 aa, synthetic construct
EVQLVESGGGLVQPGGSLRLSCAASGFTISDYWIHWVRQAPGKGLEWVAGITPAGGYTYYADSVKGRFTISADTSKNTAYLQMNSLRAEDTAVYYCARFVFFLPYAMDYWGQGTLVTVSS

SEQ ID NO: 63 - protein, 107 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQGYGAPFTFGQGTKVEIK

SEQ ID NO: 64 - protein, 120 aa, synthetic construct
EVQLVESGGGLVQPGGSLRLSCAASGFTISDYWIHWVRQEPGKGLEWVAGITPAGGYEYYADSVKGRFTISADTSKNTAYLQMNSLRAEDTAVYYCARFVFFLPYAMDYWGQGTLVTVSS

SEQ ID NO: 65 - protein, 123 aa, synthetic construct [sequence omitted]

SEQ ID NO: 66 - protein, 107 aa, synthetic construct [sequence omitted]

SEQ ID NO: 67 - protein, 123 aa, synthetic construct [sequence omitted]

SEQ ID NO: 68 - protein, 107 aa, synthetic construct [sequence omitted]

SEQ ID NO: 69 - protein, 117 aa, synthetic construct
EVQLVESGGGLVQPGGSLRLSCAASGFSINGSWIFWVRQAPGKGLEWVGAIWPFGGYTHYADSVKGRFTISADTSKNTAYLQMNSLRAEDTAVYYCARWGHSTSPWAMDYWGQGTLV

SEQ ID NO: 70 - protein, 108 aa, synthetic construct
DIQMTQSPSSLSASVGDRVTITCRASQVIRRSLAWYQQKPGKAPKLLIYAASNLASGVPSRFSGSGSGTDFTLTISSLQPEDFATYYCQQSNTSPLTFGQGTKVEIKR

SEQ ID NO: 71 - protein, 5 aa, synthetic construct
GGGGS

SEQ ID NO: 72 - protein, 6 aa, synthetic construct
DKSTGK

SEQ ID NO: 73 - protein, 4 aa, unidentified
ELLG

SEQ ID NO: 74 - protein, 10 aa, unidentified
EPKSCDKTHT

SEQ ID NO: 75 - protein, 4 aa, unidentified
CPPC

SEQ ID NO: 76 - protein, 7 aa, unidentified
PAPELLG

SEQ ID NO: 77 - protein, 104 aa, unidentified
GPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 78 - protein, 107 aa, unidentified
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 6 aa, unidentified
PAPPVA

SEQ ID NO: 80 - protein, 5 aa, unidentified
EPKSC

SEQ ID NO: 81 - protein, 10 aa, synthetic construct
GGGGSGGGGS

SEQ ID NO: 82 - protein, 15 aa, synthetic construct
GGGGSGGGGSGGGGS

SEQ ID NO: 83 - protein, 20 aa, synthetic construct
GGGGSGGGGSGGGGSGGGGS

SEQ ID NO: 84 - protein, 25 aa, synthetic construct
GGGGSGGGGSGGGGSGGGGSGGGGS

SEQ ID NO: 85 - protein, 30 aa, synthetic construct
GGGGSGGGGSGGGGSGGGGSGGGGSGGGGS

SEQ ID NO: 86 - protein, 4 aa, Homo sapiens
CPSC

SEQ ID NO: 87 - protein, 15 aa, synthetic construct
CPPCPAPGGGGPSVF

SEQ ID NO: 88 - protein, 14 aa, synthetic construct
CPPCPAPGGGPSVF

SEQ ID NO: 89 - protein, 13 aa, synthetic construct
CPPCPAPGGPSVF

SEQ ID NO: 90 - protein, 12 aa, synthetic construct
CPPCPAPGPSVF

SEQ ID NO: 91 - protein, 250 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLDKSTGKPTLYNVSLVMSDTAGTCY

SEQ ID NO: 92-99 - skipped (8 entries)

SEQ ID NO: 100 - protein, 20 aa, synthetic construct
EPKSCDKTHTCPPCPAPPVA

SEQ ID NO: 101 - protein, 17 aa, synthetic construct
ESKYGPPCPPCPAPPVA